(19) United States
(12) Patent Application Publication
(10) Pub. No.: US 2011/0059503 A1
(43) Pub. Date: Mar. 10, 2011

(54) Compositions of variant biocatalysts for preparing enantiopure amino acids

(75) Inventors: Paul P. Taylor, Arlington Heights, IL (US); Robert E. Speight, Edinburgh (GB)

(73) Assignee: Richmond Chemical Corporation

(21) Appl. No.: 12/075,044

(22) Filed: Mar. 7, 2008

Related U.S. Application Data
(60) Provisional application No. 60/905,753, filed on Mar. 8, 2007.

(51) Int. Cl. C12N 9/02; C12N 9/00; C12P 41/00

(52) U.S. Cl. 435/189; 435/183; 435/280

(57) ABSTRACT

A composition of variant biocatalysts, specifically variants of D-amino acid oxidases, with improved biocatalytic activity towards D-amino acid substrates such as, but not limited to, D-tert-leucine, D-norvaline, D-2-aminobutyrate, D-alanine, D-isoleucine, D-valine, D-methionine, D-hydroxyadamantlyglycine, D-penicillamine, or D-norleucine is disclosed. Further disclosed is a method of preparing enantioselective amino acids using variant D-amino acid oxidases of the present invention.

### CROSS-REFERENCE TO RELATED APPLICATIONS

This application claims priority to U.S. Provisional Patent Application Ser. No. 60/905,753 filed Mar. 8, 2007, the entire disclosures of which are incorporated herein by reference. Priority to this application is claimed under 35 U.S.C. §§119 and/or 120.

### BACKGROUND OF THE INVENTION

1. Field of the Invention

The present invention is generally related to the enantioselective production of amino acids using variant biocatalysts. More particularly, the present invention relates to genetically mutated variants of wild type amino acid oxidase enzymes exhibiting increased activity towards specific amino acid substrates, to produce enantiomerically pure amino acids.

2. Description of the Prior Art

Amino acids and amines in high enantiomeric purity (e.g. >99% enantiomeric excess) are of increasing industrial importance because of their applications as resolving agents, chiral auxiliaries/chiral bases and catalysts for asymmetric synthesis, particularly for pharmaceutical, agrochemical and fine chemical products. Additionally, chiral amino acids and amines possess distinct biological activity, and are therefore in demand as intermediates in the pharmaceutical and agrochemical industries. Non-proteinogenic amino acids and amines are one of the most valuable and rapidly growing classes of chemical compounds used in pharmaceutical, chemical and agrochemical discovery and development. The high value of these compounds is partly due to the difficulty of manufacturing them on a large scale. Part of this difficulty arises because many valuable amino acids and amines exist in two distinct 3-dimensional forms in a mixture that is difficult to separate, and only one form is required for a particular application.

Generally, but with the exception of glycine, each of the common amino acids has a chiral, or asymmetric, α-carbon since there are four different functional groups bonded to the α-carbon. Thus, amino acids can exist as stereoisomers, which are compounds with the same molecular formula but differ in arrangement or configuration of their atoms in space. Enantiomers are two stereoisomers, which are non-superimposable mirror images that can exist for each chiral amino acid. The mirror image pairs of amino acids are designated D, dextrorotary, or L, levorotary, depending on whether the α-carbon of the amino acid corresponds to the D- or L-enantiomer of glyceraldehyde, the common reference compound. Most naturally occurring amino acids are of the L-configuration, although a few D-amino acids occur in nature. A mixture of the two enantiomers is referred to as a racemic mixture or a racemate.

Chirality is critical to the function of compounds. In many drug applications, the FDA has mandated that only one enantiomer of a compound may be used in a particular drug, and the opposite enantiomer may not be present at all. Thus, chemical and physical methods have been sought to prepare or separate the individual enantiomers of an amino acid or amine. To meet the industrial demand for these compounds, many methods have been described and developed to prepare enantiomerically pure amino acids and amines. These methods include the physical separation or resolution of enantiomeric pairs using chromatographic or crystallization methods, biocatalytic resolution of enantiomers using enzymes, asymmetric synthesis of single enantiomers using chemo- or biological catalysts, and fermentation methods using engineered microbes. Although each of these approaches has noted advantages in specific instances, each has been limited by narrow applicability to a few families of amino acids required by the industry, and many are inherently compromised by low efficiency and relatively low yields. For example, fermentation methods are limited to the production of natural amino acids, whereas most of the amino acids required for pharmaceutical and agrochemical applications do not occur in nature and accordingly are unsuited for the complex biochemical pathways that are used in fermentative methods of production. Another approach has been to chemically manufacture amino acids and amines as racemic mixtures containing both D- and L-forms, and subsequently removing or destroying the undesired enantiomer by chemical or physical means in a process called resolution. Resolution methods are limited in almost all cases to a maximum single pass product yield of 50% thereby incurring costs and generating waste in the form of solvents and unreacted by-products. Asymmetric synthesis of amino acids using chemical and biological catalysts is often compromised by many factors including the narrow substrate ranges of the chemo- and biocatalysts used, inaccessible or expensive starting materials and stringent operating parameters for the catalysts including the need for organic solvents, chemo-catalysts, or complex methods to contain and regenerate cofactors.

As a result of these limitations, more general and robust processes have been sought for the commercial preparation of amino acids, including methods in which a chemical catalyst and a biocatalyst can be used under conditions that permit each catalyst to operate efficiently. In one such example, a process of chemo-enzymatic deracemization has been described in which a racemic mixture of a given amino acid can be converted to an optically pure single amino acid enantiomer with a theoretical yield of 100%, by a method of stereo-inversion. In this approach, a highly enantioselective oxidase biocatalyst is used to generate an imine or a keto acid intermediate from only one enantiomer of a target compound in the mixture. Amino acid oxidases and amino acid deaminases are classes of enzymes that catalyze the enantioselective oxidation of an amino acid to produce the corresponding keto acid. Enzymes that only oxidize L-amino acids are referred to as L-amino acid oxidases (or deaminases), and enzymes that only oxidize D-amino acids are referred to as D-amino acid oxidases (or deaminases). A broad specificity chemical reductant can be used simultaneously or sequentially with such a biocatalyst to reduce the resultant imine back to the amino acid. Similarly, a reducing catalyst and a source of hydrogen and ammonia can be used simultaneously or sequentially to carry out reductive amination of the keto acid back to the amino acid. In either case the intermediate is reduced in a non-stereoselective manner, thereby generating both enantiomers of the amino acid; the original enantiomer acted upon by the oxidase and its opposite enantiomer, in equal proportions. The output of this concerted oxidation and reduction process is the depletion of the enantiomer which is a substrate for the enantioselective oxidase and a concomitant increase in its opposite enantiomer; the desired product of the reaction. Using an L- or D-selective amino acid oxidase enables, respectively, an optically pure D- or L-amino acid to be prepared from a racemic mixture of amino acid enantiomers. In addition to starting with a mixture of enantiomers, it is possible, using this method, to start with an enantiomerically pure amino acid and stereoinvert it to its opposite enantiomer in a theoretical 100% yield.

This deracemization method has been shown previously to be applicable to natural amino acids, as well as to some unnatural amino acids. One of the drawbacks of the deracemization methods known in the art is that the natural, or wild type, D-amino acid oxidase biocatalysts are more restricted in their range of substrates due to their complex structures and having evolved in their native organisms to accept predominantly naturally occurring compounds. However, most industrial needs require applying the deracemization process to unnatural D-amino acids, which are poor substrates for the known wild type amino acid oxidase catalysts. Such substrates include amino acids with bulky side-chains such as, but not limited to tert-leucine, norvaline, hydroxyadamantylglycine, penicillamine, and norleucine.

The present invention provides a biocatalytic transformation process that is applicable to a diverse range of compounds, by providing a general method to develop amino acids at high optical purity. The present invention uses directed evolution to alter the substrate specificity of a wild type amino acid oxidase and significantly improve its biocatalytic activity towards amino acid substrates of choice, including sterically hindered or bulky amino acids, such as but not limited to, branched chain amino acids, halogenated amino acids, straight chain amino acids, branched alkyl groups, adamantyl groups, and functionalized amino acids. More particularly, the present invention discloses variant amino acid oxidases with increased activity towards amino acids such as but not limited to, D-tert-leucine, D-hydroxyadamantyl glycine, D-isoleucine, D-valine, D-methionine, D-alanine, D-2-aminobutyrate, D-norvaline, D-penicillamine, and D-norleucine.

Directed evolution is an iterative procedure whereby random mutagenesis of a DNA sequence encoding a protein (e.g. an enzyme) is combined with a screening or selection regime, selecting variants of the enzyme with enhanced desirable qualities such as, but not limited to, activity towards a given substrate or stability. The present invention involves the mutation of the gene that encodes a D-amino acid oxidase biocatalyst. Procedures known in the art can be used to mutate the DNA of an oxidase gene, including, but not limited to, chemical mutagenesis using agents such as nitrous acid, hydroxylamine or nitrosoguanidine (NTG), ultraviolet radiation or genetic methods such as error prone PCR or site directed or saturation mutagenesis. The present invention discloses the use of directed evolution methods that employ random, semi-random and site-directed mutagenesis, to isolate variants of D-amino acid oxidase with significantly improved biocatalytic activity towards D-amino acids that can be used in industrial processes to prepare the corresponding enantiomerically pure L-amino acids using deracemization processes. The present invention is provided to overcome certain limitations and drawbacks of the prior art, and to provide novel aspects not heretofore available.

### SUMMARY OF THE INVENTION

The present invention is directed to mutations in native, or wild type, genes encoding D-amino acid oxidase that result in novel variants of D-amino acid oxidase enzymes, with improved biocatalytic activity towards amino acid substrates of industrial relevance, particularly D-tert-leucine, D-norvaline, D-2-aminobutyrate, D-alanine, D-isoleucine, D-valine, D-methionine, D-hydroxyadamantylglycine, D-penicillamine, or D-norleucine. The present invention discloses the positions of gene mutations responsible for improved D-amino acid oxidase biocatalytic activity and the relative improvement in performance of novel variants in comparison to native, or wild type enzymes. In particular the present invention describes mutations in the D-amino acid oxidase genes of Trigonopsis variabilis, Streptomyces coelicolor, and Schizosaccharomyces pombe, and any homologous D-amino acid oxidases known in the art capable of producing variants with significantly improved biocatalytic activity towards amino acid substrates such as, but not limited to, D-tert-leucine, D-norvaline, D-2-aminobutyrate, D-alanine, D-isoleucine, D-valine, D-methionine, D-hydroxyadamantylglycine, D-penicillamine, or D-norleucine. The present invention is further directed to variant biocatalysts that are highly enantioselective oxidases capable of generating an imine and/or keto acid intermediate during stereoconversion of a racemic mixture.

A first aspect of the present invention is to provide a genetically mutated variant D-amino acid oxidase biocatalyst. The variant biocatalyst exhibits increased biocatalytic activity towards a D-amino acid substrate such as, but not limited to, D-tert-leucine, D-norvaline, D-2-aminobutyrate, D-alanine, D-isoleucine, D-valine, D-methionine, D-hydroxyadamantylglycine, D-penicillamine, or D-norleucine compared to the biocatalytic activity of the wild type enzyme. The present invention further provides increased biocatalytic activity towards branched chain amino acids, halogenated amino acids, straight chain amino acids, adamantly amino acids or functionalized amino acids.

A second aspect of the present invention is to provide a variant biocatalyst having a genetic mutation comprising at least one amino acid substitution in a region from Gly41 to Asp56 in the wild type D-amino acid oxidase of T. variabilis or a substantially homologous region in another D-amino acid oxidase.

A third aspect of the present invention is to provide a variant biocatalyst having a genetic mutation comprising at least one amino acid substitution in a region from Ile86 to Val118 in the wild type D-amino acid oxidase of T. variabilis or in a substantially homologous D-amino acid oxidase.

A further aspect of the present invention is to provide a variant biocatalyst having a genetic mutation comprising at least one amino acid substitution in a region from Ser228 to Met245 in the wild type D-amino acid oxidase of T. variabilis or in a substantially homologous D-amino acid oxidase.

Another aspect of the present invention is to provide a biocatalyst having a genetic mutation comprising at least one amino acid substitution in a region from Pro43 to Gln68 in the wild type D-amino acid oxidase of S. coelicolor or in a substantially homologous D-amino acid oxidase.

A further aspect of the present invention is to provide a biocatalyst having a genetic mutation comprising at least one amino acid substitution in a region from Leu93 to Leu113 in the wild type D-amino acid oxidase of S. coelicolor or in a substantially homologous D-amino acid oxidase.

The present invention is further directed to a method for preparing enantiopure amino acids using a variant biocatalyst of a wild type enzyme. The variant biocatalyst exhibits increased biocatalytic activity towards an amino acid substrate compared to the wild type enzyme. The method comprises providing a racemic solution of amino acid substrate starting material and contacting the racemic solution with a variant biocatalyst. The method further provides enantioselectively oxidizing a group attached to a chiral center of one enantiomer of the amino acid starting material with the variant biocatalyst to produce an imino acid or keto acid. The imino acid or keto acid is reduced, either simultaneously or sequentially, in the presence of a non-selective reductant to yield a composition comprising both enantiomers of the amino acid substrates. The non-selective reductant may be a transition metal catalyst such as, but not limited to, palladium, platinum, rhodium, ruthenium or iridium supported on platinum, alumina or other supports. Repeated cycles of this process results in a solution comprising only the enantiomer of the amino acid that is not a substrate for the oxidase biocatalyst. In one aspect of the present invention, a solution with only D-amino acid or only L-amino acids serves as the starting material. In another aspect of the present invention, a solution with only keto acids serves as the starting material if the first step in the process is reduction to produce the racemic amino acid. In another aspect of the present invention, a solution with any ratio of D-amino acid and L-amino acid serves as the starting material. In each aspect of the present invention, the product of the process is an enantiopure amino acid.

These and other aspects of the present invention will become apparent to those skilled in the art after a reading of the following description of the preferred embodiment when considered with the drawings, as they support the claimed invention.

### BRIEF DESCRIPTION OF THE DRAWINGS

FIG. 1A is a schematic of deracemization of an amino acid;

FIG. 1B is a schematic of deracemization of an amine;

FIG. 2 is a schematic of the production of Plasmid pPOT7/ScDAAO 9-1;

FIG. 3 is a schematic of the production of Plasmid pPOT9/ScDAAO 9-1;

FIG. 4 is a schematic of the production of plasmid pPOT9/SpDAAO;

FIG. 5 is a schematic of the production of plasmid pBad-Thio-TOPO/TvDAAO;

FIG. 6 is a schematic of the production of plasmid pPOT3/TvDAAO;

FIG. 7A is a graphical representation comparing the biocatalytic activity of pPOT3/TvDAAO TBG22 after heat treatment compared to pPOT3/TvDAAO TBG29 after heat treatment; and

FIG. 7B is a graphical representation comparing the biocatalytic activity of wild-type pPOT3/TvDAAO after heat treatment to variant pPOT3/TvDAAO WT1 after heat treatment.

### DETAILED DESCRIPTION

The present invention is capable of embodiments in many different forms. Preferred embodiments of the invention are disclosed with the understanding that the present disclosure is to be considered as exemplifications of the principles of the invention and are not intended to limit the broad aspects of the invention to the embodiments illustrated.

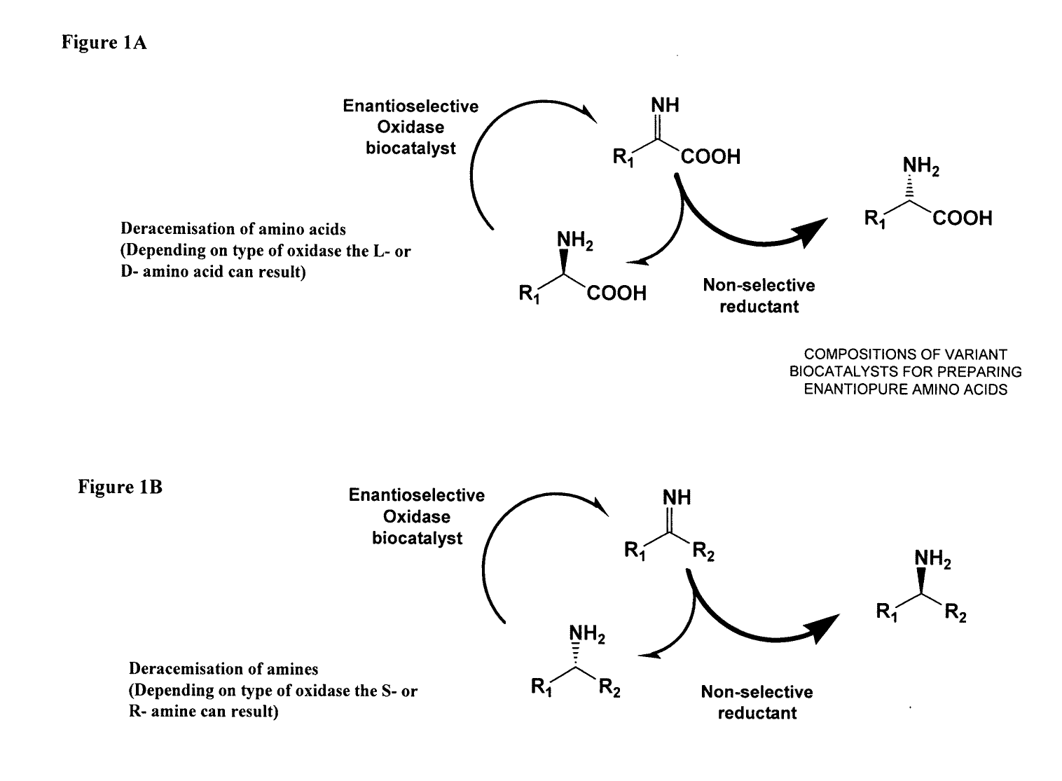

The combined use of an enantioselective amino acid oxidase biocatalyst and a non-selective reductant may be used to stereoinvert one enantiomer in a mixture of both enantiomers of an amino acid or amine as shown in FIGS. 1A and 1B respectively. This process is commonly referred to as stereoinversion if the starting material comprises only the opposite enantiomer of the desired amino acid. Furthermore, this process is referred to as deracemization if the starting material comprises a mixture of both enantiomers, in which case only the undesired enantiomer is converted in the process. In one embodiment the starting material may also be the corresponding keto acid of the desired amino acid. The resulting product may be any amino acid of the general structure RCH(NH₂)COOH. In all of these cases the process requires an amino acid oxidase biocatalyst that is highly enantioselective for either the D- or L-enantiomer of the desired amino acid. The amino acid enantiomer that results from this process depends on the enantioselectivity of the amino acid oxidase biocatalyst that is used. Use of an L-amino acid oxidase in the process results in a D-amino acid in high optical purity, and conversely, use of a D-amino acid oxidase results in a L-amino acid in high optical purity. In either case, all other aspects of the process are generally consistent as discussed in further detail below.

Amino acid oxidase biocatalysts with high enantioselectivity for either L- or D-amino acids can be isolated from natural sources, including but not limited to, micro-organisms and are considered suitable for use in the present invention. Genes encoding amino acid oxidases are known in the art and can be identified using bioinformatic methods or biological screening methods, and isolated using methods such as polymerase chain reaction (PCR). The genes encoding amino acid oxidases may be introduced into recombinant microorganisms on plasmid vectors or by chromosomal insertion into recombinant cells using well-characterized regulatory regions. Amino acid oxidase biocatalysts may be prepared by fermentation using recombinant micro-organisms including, but not limited to, Escherichia coli, Bacillus, Pseudomonas and yeast. Alternatively, amino acid oxidases can be prepared as wild type enzymes or as variants, generated by random or rational mutation of the genes encoding the enzymes. Both D- and L-amino acid oxidases are FAD dependent enzymes that can be used in a variety of formulations, including whole cells, crude cell lysates, purified enzymes or partially purified cell fractions prepared by methods such as ammonium sulfate precipitation, immobilized whole cells, or enzymes on or within solid supports.

It has been discovered that one of the major limitations of using amino acid oxidases from natural sources for amino acid stereoinversion or amino acid deracemization lies in the range of amino acid substrates that can react with the wild type oxidase biocatalysts at an industrially acceptable level. In many cases, the wild type oxidases show little or no activity towards many valuable amino acids required by industry in applications such as, but not limited to, pharmaceuticals or agrochemicals. Thus, according to the present invention, methods of mutagenesis have been applied to alter the substrate specificity of amino acid oxidases. Assays or screening methods are used to detect oxidase variants, arising by random and/or site-directed mutation that display biocatalytic activity towards substrates of interest.

In one embodiment of the present invention, a random mutagenesis approach is applied using error prone PCR to create large libraries of about 10³-10⁶ independent variant D-amino acid oxidase genes of T. variabilis, S. coelicolor, and S. pombe that are carried on plasmid vectors. These plasmid borne libraries of mutated oxidase genes are introduced into cells of E. coli K12. A colorimetric high throughput screening method is used to identify E. coli transformants that harbor mutations in the oxidase enzymes that increase the biocatalytic activity of the oxidase towards amino acid substrates, such as but not limited to D-tert-leucine, D-norvaline, D-2-aminobutyrate, D-alanine, D-isoleucine, D-valine, D-methionine, D-hydroxyadamantylglycine, D-penicillamine, or D-norleucine. This colorimetric screen has been described in the art and is a well-recognized method to obtain variants of oxidase biocatalysts.

In one embodiment of the present invention, a number of active site positions are disclosed in the DNA sequence of the wild type oxidase enzyme which, when mutated, result in oxidase variants with increased biocatalytic activity towards D-tert-leucine. These mutations, to active site residues, can increase oxidase biocatalytic activity towards other D-amino acids such as, but not limited to, D-norvaline, D-2-aminobutyrate, D-alanine, D-isoleucine, D-valine, D-methionine, D-hydroxyadamantylglycine, D-penicillamine, or D-norleucine. Significantly, the variants did not show increased activity towards L-tert-leucine, or other L-amino acids and therefore retained high enantioselectivity, which is a critical feature of oxidases for use in stereoinversion or deracemization processes. Mutations in D-amino acid oxidase that result in increased activity towards D-tert-leucine, D-norvaline, D-2-aminobutyrate, D-isoleucine, D-valine, D-hydroxyadamantylglycine, D-penicillamine, or D-norleucine are not known in the art. The present invention provides a number of variants and characterizes their activity towards D-tert-leucine and other D-amino acid substrates. Significantly, the present invention discloses examples of sequence mutations that increase S. coelicolor, T. variabilis and S. pombe D-amino acid oxidase activity towards D-tert-leucine and other D-amino acids. These mutations and the effects upon relative activity of the resulting D-AAO variants are described and shown below in Tables 1 to 7.

An amino acid substitution is the replacement or exchange of the original amino acid in a wild-type or parental protein sequence by another amino acid to produce a variant enzyme. The variant enzyme therefore has an altered protein sequence at the position of the substitution. The present invention identifies amino acid ranges in the wild-type or parental enzyme displaying increased biocatalytic activity towards D-amino acid substrates. It is understood that a number of different possible nucleotide substitutions are available to produce the same variant enzymes of the present invention, due to the degeneracy of the genetic code. It is further understood that different amino acid substitutions could be made at the same positions in the protein that have been identified in the present invention to produce variant enzymes with comparable characteristics to the variant enzymes of the present invention, and should not be limited by the examples set forth.

When the position of a mutation that influences D-amino acid oxidase activity is identified, that position can be subjected to additional mutations, to create a further library in which the native amino acid at that position in the enzyme sequence is changed to the all possible alternative amino acids, which means a further 18 amino acids in addition to the native amino acid residue and the variant which showed improved activity. This process is commonly referred to as saturation mutagenesis. For a single amino acid, the resulting library will comprise 20 variants, including the original amino acid and 19 other possibilities, including the first variant. If two residues are subjected to saturation mutagenesis then the saturation library will comprise 400 possibilities, and so forth. The present invention applies saturation mutagenesis at residues of T. variabilis, S. coelicolor, and S. pornbe D-amino acid oxidases, as shown in Tables 1-7.

The present invention contemplates use of other D-amino oxidases having homology at the protein level, as recognized by those skilled in the art, and having similar regions and constituent amino acids to those D-amino oxidases described herein. These homologous D-amino oxidases could also be targeted for mutagenesis in a similar way to the examples described herein. Homologous D-amino oxidases are defined as proteins that are not identical but have a similar three-dimensional structure, mechanism and catalytic function and can form a protein sequence alignment despite being from different species, as generally recognized by those practiced in the art. Homologous regions or amino acid positions are defined regions or positions that are spatially equivalent in said homologous D-AAOs from different species, as generally recognized by those practiced in the art. As such it is understood that D-amino acid oxidases from S. coelicolor, T. variabilis, S. pombe, R. gracilis, Fusarium solani, pig kidney, Candida biodinii and other proteins exhibiting D-amino acid oxidase homology are within the scope of the present invention. Significantly, the present invention has identified homology equivalence at the amino acid substitution Ala241 in T. variabilis D-amino acid oxidase to Thr218 in S. coelicolor D-amino acid oxidase. Additionally, the present invention has identified homology equivalence at the amino acid substitution Tyr243 in T. variabilis D-amino acid oxidase to Tyr232 in S. pombe D-amino acid oxidase. Further, the amino acid ScDAAO Gly101 may be considered equivalent to TvDAAO Ala106 and ScDAAO Ala210 may be considered equivalent to TvDAAO Ser228.

In one embodiment of the present invention, the native or variant D-amino acid oxidase biocatalyst is prepared by recombinant fermentation of E. coli using standard fed-batch fermentation protocols known in the art to achieve a high biomass. The cells are broken using mechanical means and the amino acid oxidase is recovered as an enriched, highly active and highly stable ammonium sulfate fraction. Alternatively, polyethyleneimine is added to the broken cells and the soluble fraction is recovered by centrifugation for direct use in an oxidation reaction. Oxidation of the amino acid substrate is carried out in water in a bioreactor using an aliquot of the amino acid oxidase with the substrate typically at a concentration of 50 mM to 2 M. The reaction parameters such as pH, oxygen delivery, temperature, pressure and mixing are maintained at levels that favor optimal biocatalytic activity and stability.

### EXAMPLES

### Example 1

### Cloning of the D-amino acid oxidase from S. coelicolor and isolation of variants with increased biocatalytic activity on D-amino acid substrates by random mutation and screening

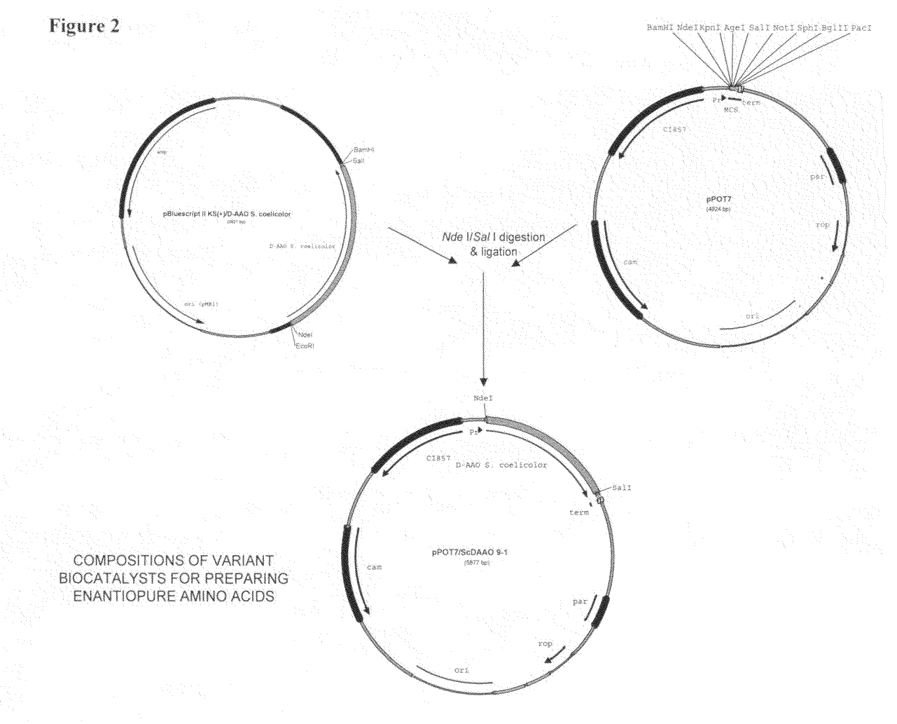

The D-amino acid oxidase gene from S. coelicolor is synthetically synthesized using standard gene synthesis techniques using the available protein sequence, Swiss-Prot Acc. # Q9X7P6. The DNA sequence, optimized for E. coli codon usage, was synthesized as identified in SEQ ID No. 1 and cloned into plasmid vector pBluescript II KS (+) using unique Eco RI and Bam HI restriction sites by Celtek Genes of Nashville, Tenn. The D-amino oxidase fragment was subsequently subcloned, using Nde I and Sal I restriction sites, into the temperature sensitive expression vector pPOT7 so that the gene is under control of the temperature inducible Lambda P_{R} promoter using standard molecular biology techniques to form the Plasmid construct pPOT7/ScDAAO, as shown in FIG. 2. E. coli strain RCI100 is transformed by this plasmid using chloramphenicol as the selectable marker and the expression of the enzyme is induced using the protocol described below.

A 15 ml sterile culture tube containing 5 ml of Luria-Bertani (LB) medium (plus 10 mg/ml chloramphenicol; Cm10) is inoculated with a single colony of RCI100 carrying Plasmid pPOT7/ScDAAO and grown overnight at 30° C. and shaking at 250 rpm. A fresh tube containing 5 ml of LB (Cm 10) is inoculated with 0.1 ml of the over night culture and the tube is incubated for 1 hr at 30° C. while shaking at 250 rpm at which time the incubator is set to 40° C. and the culture is incubated for an additional 4 hrs. The OD₆₀₀ of the culture is measured and the culture is centrifuged at 4,000 rpm in an Eppendorf™ 5801R centrifuge for 15 minutes at 4° C. The resulting cell pellet is re-suspended once in cold 50 mM phosphate buffer and centrifuged at 4,000 rpm in an Eppendorf™ 5801R centrifuge at 4° C. The pellets are lysed using Bugbuster HT Protein Extraction Reagent by Novagen™, USA according to the manufacturer's instructions. Typically, cell pellets are lysed in an amount of reagent to yield the equivalent of an OD₆₀₀ of 20. The resulting extract is then assayed using the liquid phase oxidase colorimetric assay as described below.

Liquid Phase Assay.

The liquid phase colorimetric assay is performed in a 96 well plate, in which light absorbance is measured at 510 nm, 30° C. using a VERSA_{maX} plate reader (Molecular Devices) or equivalent plate reader. The assay mixture comprises 190 μl assay solution and 10 μl enzyme solution. The assay solution (for 20 assays) comprises 2 ml 2×4-AAP (4-aminoantipyrine)/TBHBA (2,4,6-tribromo-3-hydroxybenzoic acid) mixture, 40 μl HRP solution (5 mg/ml), 1360 μl H₂O, 400 μl D-amino acid substrate solution (100 mM).

### 2×4-AAP/TBHBA Mixture (50 ml):

### 10.00 ml Potassium Phosphate (1M, pH 7.6-8.0), 1.00 ml TBHBA (2% w/v in Dimethylsulfoxide (DMSO)), 15.24 mg 4-AAP, 39.00 ml H₂O

Oxidase Activity is then Calculated, Using the Beer-Lambert Law, for Variant and Parent Enzymes As Follows:
Plate reader initial rate V₀ [mOD/min]
Extinction coefficient of colored product, ε[29,400 M⁻¹ cm⁻¹]
Path length of the assay well, 1 [cm]

### V₀¹ [μM/min]=(V₀×1000)/(ε×1)

V₀² [μmoles/min/g]=V₀¹/concentration of enzyme in assay [g/L]
V₀³ [μmoles/min/mg]=V₀²/1000
V₀⁴ [mmoles/hr/g]=V₀³×60

The native ScDAAO (pPOT7/ScDAAO) was found to have very little biocatalytic activity on D-tert-leucine or D-alanine but did have significant biocatalytic activity on D-isoleucine, D-norvaline, D-valine and D-methionine as shown in Table 1. The enzyme did not exhibit any activity towards the L-isomers of any of the amino acids tested.

TABLE 1

| DAAO | | | | Relative Activity (mmoles hr⁻¹g⁻¹ protein) | | | | | |
|---|---|---|---|---|---|---|---|---|---|
| SEQ ID | Strain | Vector/Oxidase | Mutation | D-tert-leu | D-norval | D-isoleu | D-val | D-met | D-ala |
| No. 1 | RCC0049 | pPOT7/ScDAAO | None | 0.05 | 0.90 | 3.65 | 1.52 | 0.54 | 0.05 |
| No. 6 | RCC0051 | PPOT7/ScDAAO C2 | Thr218Ile | 0.59 | 5.46 | 17.20 | 9.47 | 4.54 | 0.37 |
| No. 7 | RCC0053 | pPOT7/ScDAAO C10 | His141Tyr | 1.07 | 10.25 | 31.03 | 19.58 | 8.67 | 1.05 |
| No. 8 | RCC0054 | pPOT7/ScDAAO C18 | Val27Ile | 0.16 | 2.04 | 8.75 | 4.41 | 1.78 | 0.11 |
| No. 9 | RCC0055 | pPOT7/ScDAAO C25 | Gly101Ser | 0.18 | 3.92 | 14.46 | 9.09 | 3.18 | 0.18 |

The ScDAAO gene is mutagenized using a GeneMorph II Random Mutagenesis Kit by Stratagene™, USA based on the protocol supplied with the kit. Plasmid pPOT7/ScDAAO is used as the template at four different concentrations (500, 100, 10, and 0.1 ng), and using SEQ ID No. 2 and SEQ ID No. 3 primers. These primers are homologous to flanking regions of the cloned gene and end at the 5′ ATG start and 3′ TTA stop codon respectively. The primary mutagenesis reaction is carried using an Eppendorf Gene Mastercycler PCR machine by EppendorfrM, Germany using the manufacturers protocol (Instruction Manual #200550, program for single-block temperature cyclers).

The DNA from the primary reaction is then amplified in a secondary PCR reaction as follows:

### Secondary Amplification.

| μl | Ingredient |
|---|---|
| 28 | Molecular Biology Grade Water |
| 5 | Primary PCR reaction DNA |
| 5 | Pfu reaction buffer |
| 5 | 2 mM dNTP mix (0.05 mM each final concentration) |
| 3 | 10 μM Forward primer |
| 3 | 10 μM Reverse primer |
| 1 | Pfu Turbo polymerase |

### Program

### 1) 95° C. 2 min

### 2) 95° C. 30 sec

### 3) 55° C. 30 sec

### 4) 72° C. 1 min

5) to 2)×30 cycles

### 6) 72° C. 10 min

7) 4° C. hold
The resulting amplified DNA is purified by TAE agarose gel electrophoresis followed by extraction of the DNA from the agarose using a Qiagen Gel Extraction Kit by Qiagen™, USA. The mutagenized epPCR genes are integrated into a plasmid expression vector (pPOT7) using a modified QuikChange (Stratagene™, USA) procedure that incorporates the mutagenized epPCR genes (megaprimers) rather than the short oligonucleotide primers used in standard procedures known in the art. The product of the megaprimer QuikChange reaction is then digested with Dpn I, to remove the methylated vector template, and 2 μA is used to electroporate electro-competent RCI100. The library size is determined by plating dilutions on LB Cm 10 plates followed by incubation overnight at 30° C. Libraries are diluted to 1 ng/μl, and 1 μl is electroporated into RCI100. The electroporation libraries are plated to a cell density of 500-1000 colonies per plate, incubated over night at 25° C. followed by 6 hr at 40° C. to complete colony growth and induce expression of the ScDAAO variants. The plates are then screened using the solid phase oxidase assay.

Solid Phase Colorimetric Assay: The colonies were lifted from the transformation plates on sterile Whatman No. 1, 8.5 cm filter paper circles. Filters are frozen by dipping in liquid nitrogen for 20 seconds (or by freezing at −80° C. for 10 min) and placing on tissue paper with the colony side up to thaw. The solid phase and liquid phase colorimetric assays rely on the detection of the hydrogen peroxide by-product of a successful oxidase reaction. Solid phase assay mixture is prepared as follows:

### Solid Phase Assay Mixture (Final Volume 20 ml):

DAB tablet (Sigma, D-4418)
2 ml 1 M potassium phosphate buffer (pH 7.6)
0.1 ml 5 mg/ml horseradish peroxidase (Sigma, P6782)

### 13.9 ml H₂O

2 ml D-amino acid to be tested [100 mM]
The master mix is filtered using a 45 μM filter prior to use, then 2.2 ml of the mixture is added a Petri dish containing an 8.5 cm filter paper (Whatman, Grade 1, 1001-085). The filter papers carrying the lifted colonies are overlaid on the filter papers saturated in the assay mixture and the dish is incubated at room temperature for 2 hrs to 72 hrs to detect color. The lifted colonies are observed for up to 72 hrs and positive colonies, which are detected by an increase in brown color relative to non-mutated control, are picked and purified by re-screening again. From this screen four distinct variants with increased activity on D-tert-leucine are obtained as shown in Table 1. The variants are subjected to DNA sequencing using primers SEQ ID Nos. 4 and 5 and their DNA sequence was determined, namely SEQ ID No. 6 (ScDAAO C2), SEQ ID No. 7 (ScDAAO C 10), SEQ ID No. 8 (ScDAAO C18) and SEQ ID No. 9 (ScDAAO C25). Table 1 shows the corresponding amino acid changes and biocatalytic activity of the variants as compared to the native ScDAAO on D-tert-leucine, D-norvaline, D-isoleucine, D-valine, D-methionine and D-alanine. The variants showed a 3 to 24-fold increase in their biocatalytic activity on D-tert-leucine as compared to the native S. coelicolor oxidase as well as increased biocatalytic activity on D-norvaline, D-isoleucine, D-valine, D-methionine and D-alanine. Significantly, position Thr218 is the equivalent position to Ala241 of the TvDAAO by sequence alignment suggesting that modifications at this equivalent position in TvDAAO and other homologous DAAOs may also have similar effects on enzyme activity and specificity. Indeed, described below are variant TvDAAO enzymes that are mutated at Ala241 and display increased activity on D-tert-leucine. In addition, the present invention suggests that these variants will significantly enhance activity towards other D-amino acids, not limited to those tested herein.

### Example 2

### Isolation of D-Amino Acid Oxidase Variants of S. coelicolor with Increased Biocatalytic Activity Towards D-Amino Acid Substrates by Combining Beneficial Random Mutations

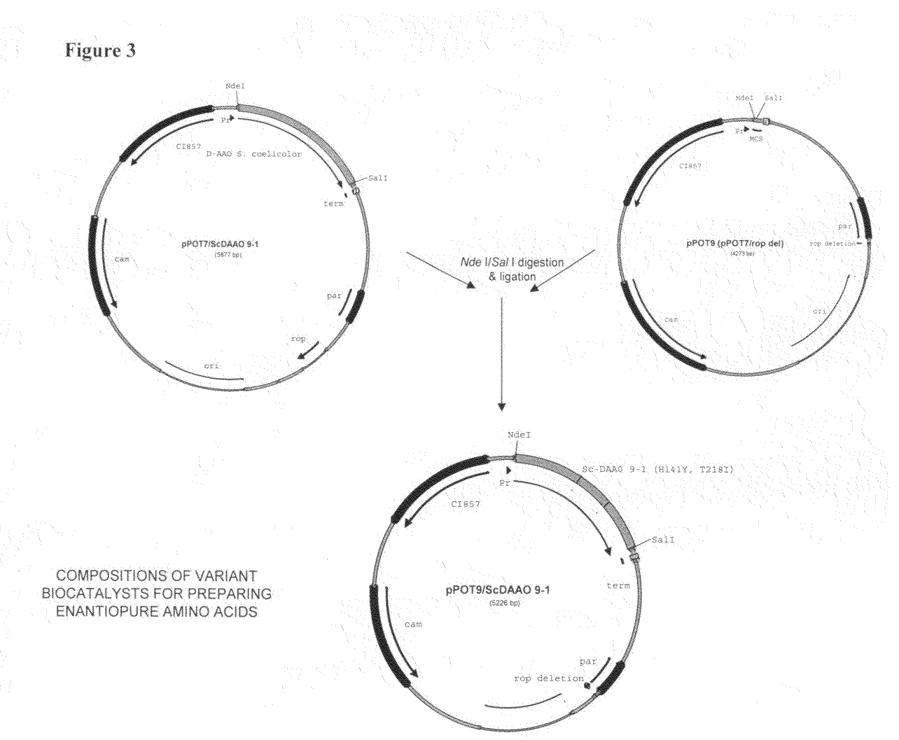

The His141Tyr and Thr218Ile mutations described in Example 1 are combined using the QuikChange procedure by Stratagene™, USA. The His141Tyr mutation is added to the Plasmid pPOT7/ScDAAO C2 variant (Thr218Ile) with a QuikChange II XL kit using the manufacturers recommended conditions, and using mutagenic primers SEQ ID No. 10 and SEQ ID No. 11. Several isolates are sequenced to confirm the correct DNA sequence, as identified in SEQ ID No. 12, and a confirmed clone, Plasmid pPOT7/ScDAAO 9-1, is analyzed for activity against several D-amino acids as described in Table 2. The results show that the two mutations were additive with regards to oxidase activity yielding a 93-fold increase in specific activity on D-tert-leucine as compared to the native S. coelicolor oxidase. Additionally, the specific activity on several other D-amino acids tested is also increased. Oxidase activity was further increased by subcloning the ScDAAO 9-1 gene onto pPOT9 using the unique Nde I and Sal I restriction sites as shown in FIG. 3. This plasmid has the rop region of pPOT7 deleted, which has been shown to increase the copy number of pBR322 derived plasmids approximately 3-fold and thus increase expression of cloned genes due to a gene dosage effect. Cloning the ScDAAO His141Thr218Ile variant into pPOT9 yielded a 1.6 fold increase in activity on D-tert-leucine as compared to the pPOT7/ScDAAO 9-1 variant. Similar increases on other D-amino acids tested were also observed as shown in Table 2.

TABLE 2

| DAAO | | | | Relative Activity (mmoles hr⁻¹g⁻¹ protein) | | | | | |
|---|---|---|---|---|---|---|---|---|---|
| SEQ ID | Strain | Vector/Oxidase | Mutation | D-tert-leu | D-norval | D-isoleu | D-val | D-met | D-ala |
| No. 1 | RCC0049 | pPOT7/ScDAAO | None | 0.05 | 0.90 | 3.65 | 1.52 | 0.54 | 0.05 |
| No. 12 | RCC0058 | pPOT7/ScDAAO 9-1 | His141Tyr, | 4.19 | 20.45 | 45.76 | 36.36 | 18.63 | 3.51 |
| | | | Thr218Ile | | | | | | |
| No. 1 | RCC0074 | pPOT9/ScDAAO | None | 0.07 | 1.26 | 6.48 | 3.20 | 1.06 | 0.04 |
| No. 12 | RCC0063 | pPOT9/ScDAAO 9-1 | His141Tyr, | 6.91 | 34.78 | 40.03 | 59.37 | 27.84 | 4.67 |
| | | | Thr218Ile | | | | | | |

### Example 3

### Isolation of D-Amino Acid Oxidase Variants with Increased Specific Activity to D-Amino Acid Substrates and Increased Thermal Stability by Random Mutagenesis of S. coelicolor Variant HIS141Tyr, Thr218IlE (pPOT9/ScDAAO 9-1)

Plasmid pPOT9/ScDAAO 9-1 is subject to mutagenesis using the GeneMorph II Random Mutagenesis kit. The mutagenesis, screening, DNA sequencing and specific activity determination were performed as described in Example 2 with one exception. To decrease the background of active colonies, the library colony lifts were heated at 55° C. for 90 minutes prior to activity screening. These conditions completely inactivate the oxidase activity in induced colonies of RCI100 carrying Plasmid pPOT9/ScDAAO 9-1. Thus, any variants having biocatalytic activity after heat treatment should be more active and/or more heat stable and yield a more robust oxidase. As shown in Table 3, a number of improved variants exhibiting higher specific biocatalytic activity on D-tert-leucine were obtained (1.1 to 1.8 fold increase in activity over the His141Tyr, Thr218Ile double mutant and 189 fold more than the native enzyme) as well as having increased thermal stability compared to the native enzyme. The present invention suggests further improvements made by additional rounds of mutagenesis coupled with more stringent screening.

TABLE 3

| | | | | Relative Activity (mmoles hr⁻¹g⁻¹ protein) | | | | | |
|---|---|---|---|---|---|---|---|---|---|
| DAAO | | | | D-tert- | D- | | | | |
| SEQ ID | Strain | Vector/Oxidase | Mutations | leu | norval | D-isoleu | D-val | D-met | D-ala |
| No. 1 | RCC0074 | pPOT9/ScDAAO | None | 0.07 | 1.26 | 6.48 | 3.20 | 1.06 | 0.04 |
| No. 12 | RCC0063 | pPOT9/ScDAAO | His141Tyr Thr218Ile | 6.91 | 34.78 | 40.03 | 59.37 | 27.84 | 4.67 |
| | | 9-1 | | | | | | | |
| No. 13 | RCC0064 | pPOT9/ScDAAO | His141Tyr Thr218Ile | 9.60 | 50.38 | 82.89 | 83.83 | 37.49 | 7.46 |
| | | 1-3-45 | Asp7Glu | | | | | | |
| No. 14 | RCC0066 | pPOT9/ScDAAO | His141Tyr Thr218Ile | 9.55 | 42.52 | 70.17 | 72.90 | 38.21 | 9.56 |
| | | 2-4-2 | Glu62Lys | | | | | | |
| No. 15 | RCC0068 | pPOT9/ScDAAO | His141Tyr Thr218Ile | 9.99 | 39.93 | 59.18 | 64.38 | 45.03 | 9.13 |
| | | 2-4-12 | Gln68Arg | | | | | | |
| No. 16 | RCC0069 | pPOT9/ScDAAO | His141Tyr Thr218Ile | 11.93 | 69.58 | 101.56 | 110.43 | 74.03 | 11.80 |
| | | 1-26 | Val50Ala | | | | | | |
| No. 17 | RCC0073 | pPOT9/ScDAAO | His141Tyr Thr218Ile | 12.91 | 64.67 | 102.26 | 103.34 | 57.00 | 9.61 |
| | | 4-37 | Leu93Val | | | | | | |
| No. 18 | RCC0079 | pPOT9/ScDAAO | His141Tyr Thr218Ile | 10.53 | 68.04 | 125.70 | 112.20 | 83.00 | 7.34 |
| | | 1-29 | Glu99Gly | | | | | | |
| No. 19 | RCC0081 | pPOT9/ScDAAO | His141Tyr Thr218Ile | 7.89 | 43.42 | 82.65 | 76.42 | 36.37 | 6.15 |
| | | 1-3-31 | Ala211Thr | | | | | | |
| No. 20 | RCC0085 | pPOT9/ScDAAO | His141Tyr Thr218Ile | 12.55 | 68.47 | 108.59 | 112.20 | 36.34 | 10.14 |
| | | 4-22 | Trp108Arg | | | | | | |

### Example 4

### Isolation of D-Amino Acid Oxidase Variants with Increased Specific Activity and Thermal Stability to D-Amino Acid Substrates by Random Mutagenesis of S. coelicolor Variant HIS141Tyr, Thr218Ile, Val50A (pPOT9/ScDAAO 1-26)

Variant pPOT9/ScDAAO 1-26 (SEQ ID No. 16) is subject to further mutagenesis using the GeneMorph II Random Mutagenesis kit (Stratagene, CA). The mutagenesis, screening, DNA sequencing and specific activity determination are performed as described in example 4 except the screening stringency was increased to 2.5 hr at 60° C. Under these conditions the activity of the enzyme from RCC0069 (pPOT9/ScDAAO 1-26) is completely inactivated. Colonies exhibiting activity after this treatment are assayed as previously described. After DNA sequencing the exact amino acid changes were determined as shown in Table 4. Several of these variants exhibited increased specific activity on D-tert-leucine, the most significant improvement being RCC00127 (Ala111Gly; SEQ ID No. 28) which showed a 294-fold increase in activity over the native ScDAAO. To test the thermal stability the extracts are incubated at 50° C. for 1 hr in an Eppendorf thermocycler, after centrifugation at 12,000 rpm for 1 min the activity of the heat treated extract on D-tert-leucine was determined. Table 4 shows that all of the selected variants had more remaining activity than the controls RCC0063 (SEQ ID No. 12) and RCC0069 (SEQ ID No. 16), with three of the variants (SEQ ID No's 21, 28 and 32) having about 50% of their specific activity remaining after this treatment.

TABLE 4

| | | | | D-cert-leu | % Activity |
|---|---|---|---|---|---|
| DAAO | | | Additional | mmoles hr⁻¹g⁻¹ | remaining after |
| SEQ ID | Strain | Vector/Oxidase | Mutations | protein | 1 hr at 50° C. |
| No. 12 | RCC0063 | pPOT9/ScDAAO 9-1 | None¹ | 6.91 | 0.0 |
| | | (control) | | | |
| No. 16 | RCC0069 | pPOT9/1-26 | None² | 10.39 | 4.5 |
| No. 21 | RCC0118 | pPOT9/1-26-A11 | Gly18Ser² | 8.21 | 50.1 |
| No. 22 | RCC0119 | pPOT9/1-26-A20 | Arg60Leu² | 7.34 | 15.8 |
| No. 23 | RCC0120 | pPOT9/1-26-A21 | Gly216Ser² | 12.38 | 14.9 |
| No. 24 | RCC0121 | pPOT9/1-26-A33 | Ser49Ala | 18.44 | 22.8 |
| | | | Ala210Thr² | | |
| No. 25 | RCC0122 | pPOT9/1-26-A37 | Thr100Met | 21.82 | 32.7 |
| | | | Ala210Thr² | | |
| No. 26 | RCC0123 | pPOT9/1-26-A47 | Ala211Thr² | 17.22 | 10.3 |
| No. 27 | RCC0124 | pPOT9/1-26-A54 | Leu93Val² | 17.80 | 33.4 |
| No. 28 | RCC0126 | pPOT9/1-26-A61 | Asp158Tyr² | 16.05 | 54.2 |
| No. 29 | RCC0127 | pPOT9/1-26-B70 | Ala111Gly² | 27.62 | 17.3 |
| No. 30 | RCC0128 | pPOT9/1-26-B83 | Leu129Val² | 12.59 | 17.1 |
| No. 31 | RCC0129 | pPOT9/1-26-B87 | Gly101Ser² | 8.86 | 18.8 |
| | | | Asp106Val | | |
| No. 32 | RCC0130 | pPOT9/1-26-B89 | Glu8Val | 9.67 | 48.4 |
| | | | Gly18Ser² | | |
| No. 33 | RCC0131 | pPOT9/1-26-B100 | Phe221Tyr² | 13.52 | 10.8 |
| No. 34 | RCC0132 | pPOT9/1-26-B120 | Gly101Ser | 15.57 | 21.6 |
| | | | Leu113Ile² | | |
| No. 35 | RCC0133 | pPOT9/1-26-B127 | Ala210Thr² | 14.84 | 22.1 |

1-Variant carries the His141Tyr, Thr218Ile mutations
2-Variant also carries the Val50Ala, His141Tyr, Thr218Ile mutations

### Example 5

### Cloning of the Schizosaccharomyces pombe D-Amino Acid Oxidase and Isolation of Variants by Saturation Mutagenesis at Tyrosine 232

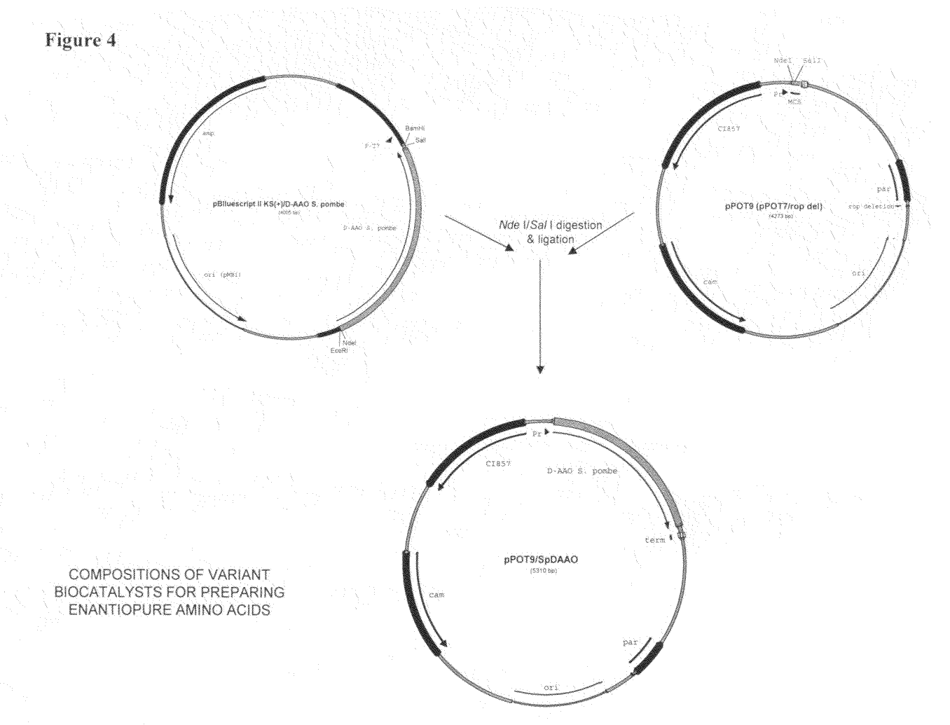

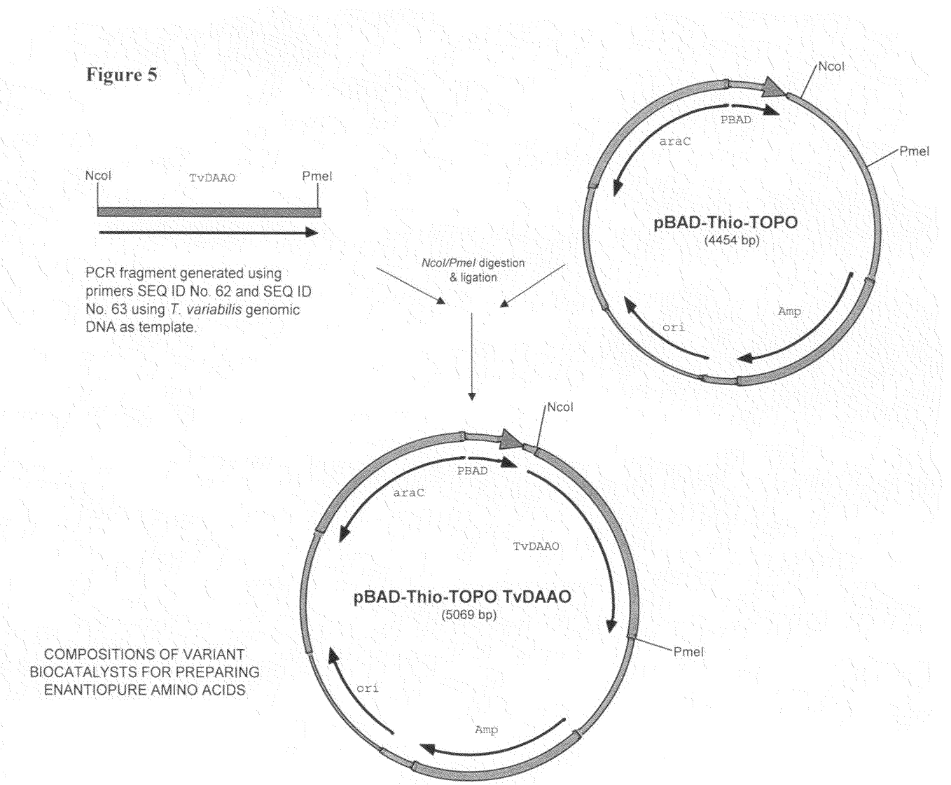

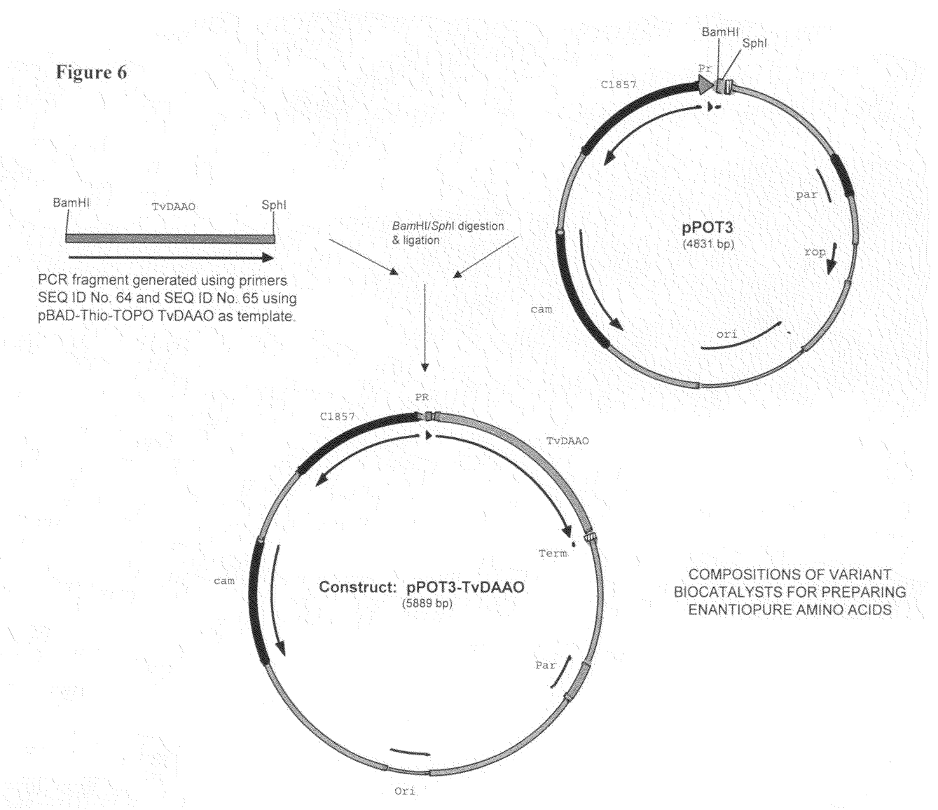

The D-amino acid oxidase gene from S. pombe is synthetically synthesized using standard gene synthesis techniques using the available protein sequence (Swiss-Prot Acc. # Q9Y7N4). The DNA sequence, optimized for E. coli codon usage, is synthesized and cloned into plasmid vector pBluescript II KS (+) using unique Eco RI and Bam HI restriction sites by Celtek Genes, Nashville, Tenn. (SEQ ID No. 36). The D-amino oxidase fragment is subsequently subcloned using Nde I and Sal I restriction sites into the temperature sensitive expression vector pPOT9 so that the gene was under control of the temperature inducible Lambda P_{R} promoter using standard molecular biology techniques to construct plasmid pPOT9/SpDAAO as shown in FIG. 4.

Primers are designed, as identified by SEQ ID Nos. 37 and 38 so that Tyr232, equivalent to positions Tyr243 in T. variabilis D-AAO and Tyr220 in S. coelicolor D-AAO, is randomized so that all the possible amino acid substitutions are possible at this position. Saturation mutagenesis is carried out using the QuikChange procedure as previously described by Stratagene™, USA. Fifty random colonies are analyzed by DNA sequencing and 90% of the isolates are found to be variants at position Tyr232. From this, variants carrying 17 out of the possible 19 different amino acid substitutions are obtained. The degenerate libraries are screened using the solid phase oxidase screen and several oxidase positive colonies are isolated and their DNA sequences determined. The positive variants are induced and their specific activity is determined using the liquid oxidase assay as previously described and shown in Table 5. The substitutions having a positive effect on activity on D-tert-leucine (in decreasing order of activity) are alanine, histidine, cysteine, glutamine, glycine, isoleucine and serine. Substitution with methionine did not affect the activity on D-tert-leucine, but did have a beneficial affect on the oxidation of D-norvaline and is unusual in that it was less effective on all the amino acids tested. Generally substitutions at this site tended to decrease the resultant DAAO on D-alanine, D-isoleucine and D-valine while increasing activity on D-tert-leucine, D-norvaline and D-methionine (see exceptions in Table 5). These results could be reasonably expected since the variants selected worked well on the branched chain amino acids as seen with the ScDAAO in above (Examples 2, 3 and 4). The present invention suggests further testing of these variants on other natural and unnatural D-amino acids may reveal many other useful and novel oxidase reactions. Further improvements are within the scope of the present invention by combining some of these amino acid substitutions or by carrying out additional rounds of mutagenesis followed by more stringent screening, i.e. increased time at the same temperature or a higher temperature.

TABLE 5

| | | | | Relative Actvity (mmoles hr⁻¹g⁻¹ protein) | | | | | |
|---|---|---|---|---|---|---|---|---|---|
| DAAO | | | | | D- | | | D- | |
| SEQ ID | Strain | Vector/Oxidase | Mutations | D-tert-leu | norval | D-isoleu | D-val | met | D-ala |
| No. 36 | RCC0087 | pPOT9/SpDAAO native | none | 0.14 | 3.53 | 4.25 | 15.96 | 3.88 | 36.31 |
| No. 39 | RCC0096 | pPOT9/SpDAAO 4A-2 | Tyr232Gly | 0.19 | 8.38 | 1.77 | 1.33 | 11.62 | 0.68 |
| No. 40 | RCC0099 | pPOT9/SpDAAO 4A-6 | Tyr 232His | 0.69 | 2.71 | 2.46 | 3.65 | 1.18 | 2.68 |
| No. 41 | RCC0102 | pPOT9/SpDAAO 4A-16 | Tyr 232Ile | 0.18 | 3.64 | 0.48 | 0.74 | 1.95 | 0.59 |
| No. 42 | RCC0104 | pPOT9/SpDAAO 4A-19 | Tyr 232Ser | 0.17 | 13.80 | 2.02 | 1.35 | 10.06 | 0.76 |
| No. 43 | RCC0106 | pPOT9/SpDAAO 4A-23 | Tyr 232Met | 0.14 | 25.66 | 3.08 | 4.54 | 10.92 | 5.52 |
| No. 44 | RCC0111 | pPOT9/SpDAAO 4A-47 | Tyr 232Gln | 0.33 | 15.65 | 2.95 | 6.88 | 11.58 | 5.32 |
| No. 45 | RCC0112 | pPOT9/SpDAAO 6A-7 | Tyr 232Cys | 0.58 | 34.58 | 13.59 | 8.78 | 27.21 | 5.92 |
| No. 46 | RCC0114 | pPOT9/SpDAAO 6B-10 | Tyr 232Ala | 1.09 | 25.05 | 9.65 | 9.60 | 27.94 | 2.88 |

### Example 6

### Isolation of T. variabilis D-Amino Acid Oxidase Variants by Saturation Mutagenesis at Positions which Influence Activity Towards D-Tert-Leucine and D-Norvaline

The gene encoding T. variabilis D-amino acid oxidase was isolated by PCR using genomic DNA and oligonucleotide primers DAAO Nterm NcoI (SEQ ID No. 62) and DAAO Cterm PmeI (SEQ ID No. 63), which were designed to amplify the whole gene and remove the 5′ intron as well as to introduce a 5′ Nco I restriction site and a 3′ Pme I restriction site. The gene was then ligated with the pBAD/Thio-TOPO plasmid (Invitrogen) using the same restriction sites and standard methods. The gene was then amplified by PCR using the oligonucleotide primers DAAO Nterm BamHI (SEQ ID No. 64) and DAAO Cterm SphI (SEQ ID No. 65) to introduce 5′ Bam HI and 3′ Sph I restriction sites for subcloning with the pPOT3 vector to produce pPOT3/TvDAAO.

Saturation mutagenesis of the gene encoding T. variabilis D-amino acid oxidase was carried out at positions Ala241 and Tyr243 in the amino acid sequence, based upon the observation herein that mutations at these positions had resulted in improved oxidase activity towards D-tert-leucine and/or D-norvaline in other D-amino acid oxidases such as S. coelicolor and S. pombe. Saturation mutagenesis was carried out using the QuikChange Kit by Stratagene™ according to the manufacturer's protocol. Using the genetic code and standard methods, degenerate forward and reverse oligonucleotide PCR primers were designed to introduce all 400 possible amino acid combinations at positions 241 and 243. The resulting library was then screened using the solid phase oxidase colorimetric assay as described below.

The plasmid borne libraries were used to transform E. coli BW22513 by electroporation and the transformants were plated to a density of 500-1000 colonies per plate, incubated over night at 25° C. followed by 6 hr at 40° C. to complete colony growth and induce the T. variabilis DAAO variants. The plates can then be then screened using a solid phase oxidase assay as described above in example 1.

A number of variants were obtained with significantly enhanced biocatalytic activity on D-tert-leucine, with three examples presented here named pPOT3/TvDAAO m21, TBG21 and TBG22, as shown in Table 6. Other variants with improved activity against D-tert-leucine had Ala241 substituted by Ile, Pro and Leu as well as His and Met as found in pPOT3/TvDAAO TBG21 and TBG22 respectively. In the same way, Tyr243 could also be substituted by Ile, His and Phe. The present invention suggests that these mutations will significantly enhance enantioselectivity on numerous other D-amino acids not limited to those tested herein. These variants can be used as a starting point for further enhancement, using iterative mutagenesis, on the amino acids tested or other natural and unnatural D-amino acids targets.

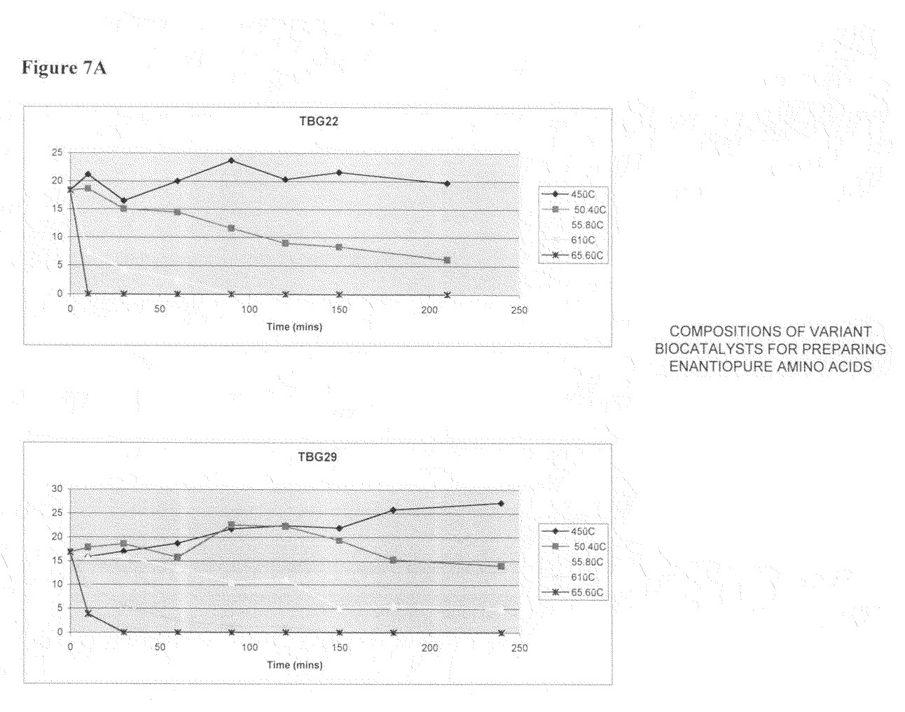

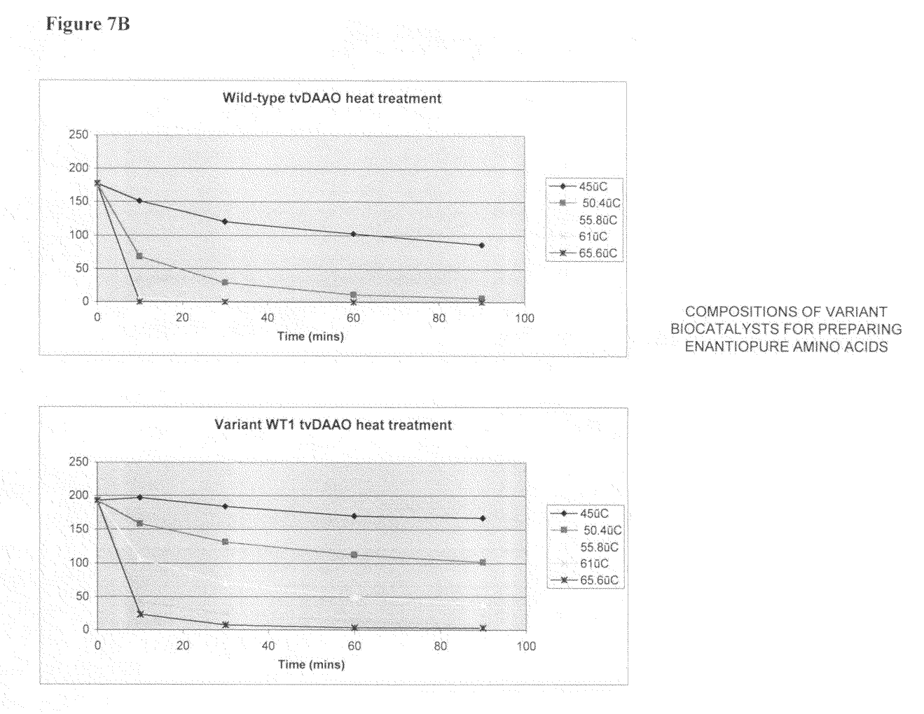

The variant pPOT3/TvDAAO TBG22 was chosen as the template for further iterative mutagenesis. Sequence alignment of various DAAO protein sequences demonstrates that the equivalent position of His141 of S. coelicolor DAAO, which was shown to improve the activity of this enzyme towards D-tert-leucine herein, in TvDAAO is Thr148. Saturation mutagenesis and screening as described above against D-tert-leucine revealed that the TvDAAO Thr148Pro substitution provided increased activity against D-tert-leucine and increased thermostability. Furthermore, the substitutions Met156Leu and Met209Leu are known to offer stabilization against hydrogen peroxide damage. These mutations were also incorporated into the pPOT3/TvDAAO TBG22 Thr148Pro variant to product the variant pPOT3/TvDAAO TBG29. This variant had improved activity against D-tert-leucine (Table 6) and also increased thermostability compared to pPOT3/TvDAAO TBG22, as shown in FIG. 7A. The thermostability was tested by heating aliquots of a solution of the enzyme in a heating block at various temperatures. At various time points, an aliquot was removed from the heat block and placed on ice. The aliquots were then assayed using the liquid colorimetric assay as described above to assess the amount of oxidase activity remaining in the sample. The initial oxidation rate observed [mOD/min] is directly proportional to the amount of active enzyme remaining in solution.

TABLE 6

| DAAO | | | | Relative Activity | | |
|---|---|---|---|---|---|---|
| SEQ | | | | (mmoles hr⁻¹g⁻¹ protein) | | |
| ID | Strain | Vector/Oxidase | Mutation | D-tert-leu | D-2-aba | D-ala |
| No. 47 | RCC1001 | pPOT3/TvDAAO wt | None | 1.1 | 1995.7 | 1164.7 |
| No. 48 | RCC1011 | pPOT3/TvDAAO m21 | Tyr243His | 5.7 | 151.0 | 18.8 |
| No. 49 | RCC1021 | pPOT3/TvDAAO | Ala241His Tyr243Val | 38.9 | 267.9 | 95.6 |
| | | TBG21 | | | | |
| No. 50 | RCC1022 | pPOT3/TvDAAO | Ala 241Met | 46.0 | 682.5 | 109.8 |
| | | TBG22 | Tyr243Val | | | |
| No. 51 | RCC1029 | pPOT3/TvDAAO | Ala 241Met | 59.1 | 620.3 | 153.9 |
| | | TBG29 | Tyr243Val Thr148Pro | | | |
| | | | Met 156Leu | | | |
| | | | Met209Leu | | | |
| No. 52 | RCC1039 | pPOT3/TvDAAO | Ala 241Met | 46.9 | 695.4 | 212.1 |
| | | TBG39 | Tyr243Val Thr148Pro | | | |
| | | | Met156Leu | | | |
| | | | Met209Leu Gly41Arg | | | |
| | | | Ala 106Thr | | | |
| No. 53 | RCC1040 | pPOT3/TvDAAO | Ala 241Met | 111.7 | 810.0 | 270.8 |
| | | TBG40 | Tyr243Val Thr148Pro | | | |
| | | | Met156Leu | | | |
| | | | Met209Leu Gly41Arg | | | |
| | | | Ala 106Thr | | | |
| | | | Tyr55Ser | | | |
| No. 54 | RCC1045 | pPOT3/TvDAAO | Ala 241Met | 79.2 | 617.1 | 204.3 |
| | | TBG45 | Tyr243Val Thr148Pro | | | |
| | | | Met156Leu | | | |
| | | | Met209Leu Gly41Arg | | | |
| | | | Ala 106Thr | | | |
| | | | Tyr55Ser Asp56Glu | | | |
| No. 55 | RCC1047 | pPOT3/TvDAAO | Ala 241Met | 76.2 | 641.9 | 278.2 |
| | | TBG47 | Tyr243Val Thr148Pro | | | |
| | | | Met156Leu | | | |
| | | | Met209Leu Gly41Arg | | | |
| | | | Ala 106Thr | | | |
| | | | Tyr55Ser Asp56Glu | | | |
| | | | Ala103Pro | | | |

Further random mutagenesis was performed using pPOT3/TvDAAO TBG22 and TBG26 as the templates. Libraries of randomly mutated variants were produced using the GeneMorph II Random Mutagenesis kit (Stratagene, CA) and screened for increased heat stability as described above.

The mutations Gly41Arg and Ala106Thr were identified as providing increased activity and thermostability using the solid phase plate screen. These mutations were incorporated into the pPOT3/TvDAAO TBG29 template using the Quikchange procedure to produce the variant pPOT3/TvDAAO TBG39. In addition, it is noted that Ala106 of TvDAAO is in alignment with Gly101 of S. coelicolor, which was found herein to offer increased activity when mutated to Ser. This evidence further establishes the way that similar mutations in homologous proteins can be expected to have similar effects, even if the specific oxidase has not been studied previously.

Further screening of the random epPCR libraries yielded a number of variants that were identified with increased activity and/or stability. Three of the most active in the screen (darkest colonies) had the mutations Tyr55Ser, Asp56Glu and Ala103Pro. These mutations were introduced into the variant pPOT3/TvDAAO TBG39 using the QuikChange protocol and are named pPOT3/TvDAAO TBG40, pPOT3/TvDAAO TBG45 and pPOT3/TvDAAO TBG47 for the incorporation of Tyr55Ser, Tyr55Ser/Asp56Glu and Tyr55Ser/Asp56Glu/Ala103Pro respectively. Furthermore, the mutations Asp56Pro and Tyr55Thr were also found to offer similar advantages. Other mutations yielding variants with increased activity and/or stability but were weaker in the screen than those described above include Gly41Ala, Ala51Thr, Asp64Asn, Glu73Lys, Ser77Asn, Cys108Ser, Met156Ile, Asp188Ala, Lys225Asn and Ser228Thr. It would be expected that combinations of these mutations or the corresponding positions in other homologous DAAOs would lead to variants with improved activity, specificity and/or stability.

### Example 7

### Construction of the Variant pPOT3/TvDAAO WT1

This variant was named pPOT3/TvDAAO WT1 and was produced using the QuikChange procedure and the pPOT3/TvDAAO TBG39 template to change Met241 and Val243 back to the wild type amino acids (Ala241 and Tyr243). The resulting variant pPOT3/TvDAAO WT1 was tested for improved heat stability as shown in FIGS. 7A and 7B. The variant pPOT3/TvDAAO WT1 displayed significantly improved thermostability compared to the wild-type protein and retained activity at higher temperatures and for longer periods, with activity still detected after 10 minutes at 65.6° C., whereas the wild-type enzyme was completely inactivated at 55.8° C. before the first time point. The variant pPOT3/TvDAAO WT1 displays the same substrate specificity as the wild-type enzyme but has significantly increased thermostability leading to a more robust biocatalyst.

Detailed structural modeling to further examine the TvDAAO enzyme identified Ser228 as a possible position in TvDAAO for mutagenesis to modulate substrate specificity. The Ser228 position of TvDAAO was subjected to saturation mutagenesis as described above using the gene encoding the enzyme pPOT3/TvDAAO WT1 as the template as pPOT3/TvDAAO WT1 displayed low activity against D-tert-leucine. The library was screened against D-tert-leucine using the solid phase colorimetric assay as described above. The substitutions Ser228Trp, Ser228Tyr and Ser228Leu in addition to the pPOT3/TvDAAO WT1 mutations, named pPOT3/TvDAAO TBG59, TBG60 and TBG63 respectively, were found to significantly increase the activity against D-tert-leucine to a level comparable with pPOT3/TvDAAO TBG47 (Table 7). In addition, the substitution Ser228Phe was found to exhibit similar activity.

The Ser228 position of TvDAAO was subjected to saturation mutagenesis as described above using the gene encoding the enzyme pPOT3/TvDAAO TBG47 as the parent. The library was screened against D-tert-leucine using the solid phase colorimetric assay as described above. The substitutions Ser228Ile and Ser228Met in addition to the pPOT3/TvDAAO TBG47 mutations, named pPOT3/TvDAAO TBG64 and TBG65 respectively, were found to have similar activity against D-tert-leucine compared to the pPOT3/TvDAAO TBG47 parent (Table 7).

Enzyme variants were also assayed against D-hydroxyadamantyl glycine (Table 7). Although the overall activity against D-hydroxyadamantyl glycine was lower than against D-tert-leucine the variants displayed significantly higher activity than the TvDAAO WT1 enzyme. This result demonstrates that the amino acid substitutions that led to increased D-tert-leucine activity also increased by about five-fold the oxidase activity against D-hydroxyadamantyl glycine compared to the wild type enzyme.

TABLE 7

| | | | | Relative Activity | |
|---|---|---|---|---|---|
| | | | | (mmoles hr⁻¹g⁻¹ protein) | |
| | | | | | D-hydroxy- |
| DAAO | | | | | adamantyl |
| SEQ ID | Strain | Vector/Oxidase | Mutation | D-tert-leu | glycine |
| No. 56 | RCC1002 | pPOT3/TvDAAO WTI | Thrl48Pro Met156Leu | 0.02 | 0.01 |
| | | | Met209Leu Gly4lArg | | |
| | | | Ala106Thr | | |
| No. 55 | RCC1047 | pPOT3/TvDAAO TBG47 | Ala241Met Tyr243Val | 1.69 | 0.05 |
| | | | Thr148Pro Met156Leu | | |
| | | | Met209Leu Gly4lArg | | |
| | | | Ala106Thr Tyr55Ser | | |
| | | | Asp56Glu Ala103Pro | | |
| No. 57 | RCC1059 | pPOT3/TvDAAO TBG59 | Thr148Pro Met156Leu | 1.87 | Not done |
| | | | Met209Leu Gly4lArg | | |
| | | | Ala106Thr Ser228Trp | | |
| No. 58 | RCC1060 | pPOT3/TvDAAO TBG60 | Thr148Pro Met156Leu | 1.60 | Not done |
| | | | Met209Leu Gly41Arg | | |
| | | | Ala106Thr Ser228Tyr | | |
| No. 59 | RCC1063 | pPOT3/TvDAAO TBG63 | Thr148Pro Met156Leu | 1.59 | 0.06 |
| | | | Met209Leu Gly41Arg | | |
| | | | Ala106Thr Ser228Leu | | |
| No. 60 | RCC1064 | pPOT3/TvDAAO TBG64 | Ala241Met Tyr243Val | 1.52 | 0.06 |
| | | | Thr148Pro Met156Leu | | |
| | | | Met209Leu Gly41Arg | | |
| | | | Ala106Thr Tyr55Ser | | |
| | | | Asp56Glu Ala103Pro | | |
| | | | Ser228Ile | | |
| No. 61 | RCC1065 | pPOT3/TvDAAO TBG65 | Ala241Met Tyr243Val | 1.29 | 0.06 |
| | | | Thr148Pro Met156Leu | | |
| | | | Met209Leu Gly41Arg | | |
| | | | Ala106Thr Tyr55Ser | | |
| | | | Asp56Glu Ala103Pro | | |
| | | | Ser228Met | | |

The results of the TvDAAO mutagenesis demonstrate that incorporating mutations that increase the specificity towards D-tert-leucine also decrease the specificity towards D-alanine. This result was also the case for the SpDAAO enzyme. This reduction in D-alanine activity is of particular significance for the expression of the enzymes in E. coli where D-alanine is required for cell wall biosynthesis. Therefore, the toxic effect of oxidase expression is reduced when variants with reduced D-alanine activity are expressed, leading to higher cellular biomass and protein yields. Furthermore, variants with improved thermostability are expressed to higher levels as there is less oxidase degradation during the fermentation or downstream processing.

It is noted that the data presented herein identifies certain trends and specific regions of the protein that enhance the activity and stability of a family of oxidase proteins. As discussed above, mutations of proteins, and substantially similar homologous proteins, in these regions should offer similar benefits and these regions constitute the results of the present invention.

For example, one region is encompassed by the amino acids Gly41 to Asp56 in TvDAAO, which includes the specific amino acids Gly41, Tyr55 and Asp56 that have been mutated in this work and shown to be beneficial. The equivalent region in S. coelicolor DAAO is Pro43 to Gln68 and includes the amino acids Val50, Arg60, Glu62 and Gln68 that have been mutated in this work and shown to be beneficial.

A second region that has been shown to have a strong effect on oxidase stability is encompassed by the amino acids Ile86 to Val118 in TvDAAO, which includes the specific amino acids Ala103 and Ala106 that have been mutated in this work and shown to be beneficial. The equivalent region in S. coelicolor DAAO is Leu93 to Leu113 and includes the amino acids Leu93, Glu99, Thr100, Gly101, Asp106, Trp108, Ala111 and Leu113 that have been mutated in this work and shown to be beneficial.

In general, the mutations and regions described herein are unique, novel and could not be predicted. Additionally, literature reports of mutations in TvDAAO have focused either on stability improvements or biochemical analyses rather than substrate specificity modulation as described herein. Furthermore, the published crystal structures of the pig kidney and R. gracilis enzymes have identified the active site amino acids in these enzymes which naturally suggest important amino acids that could be targeted for mutation. However, these rational approaches often do not produce the expected results and a multi-faceted approach as described herein is necessary.

### Example 8

### Preparation of an Oxidase Biocatalyst

A culture of recombinant E. coli containing a cloned gene, encoding a suitable amino acid oxidase biocatalyst, such as Trigonopsis variabilis D-amino acid oxidase, is fermented in a growth medium prepared as follows: Stock Salts Solution: 10 g/l (NH₄)₂SO₄, 73 g/l K₂HPO₄, 18 g/l NaH₂PO₄.2H₂O, 2.5 g/l (NH₄)₂ Hydrogen-Citrate are mixed and autoclaved. Trace Elements Stock: 0.5 g/l CaCl₂.2H₂O, 10.03 g/l FeCl₃, 0.18 g/l ZnSO₄.7H₂O, 0.16 g/l CuSO₄.5H₂O, 0.15 g/l MnSO₄.H₂O, 0.18 g/l CoCl₂.6H₂O, 22.3 g/l Na₂EDTA.2H₂O are mixed and autoclaved. A 100 ml volume of growth medium is prepared comprising 20 ml of Stock Salts Solution, 0.2 ml of Trace Elements Stock, 0.2 ml of sterile 1 M MgSO₄.7H₂O solution, 2 ml sterile 50% Glucose solution, and appropriate amounts of required antibiotic, sterile water to 100 ml in a 0.5 L flask. The culture is incubated at 28° C. with agitation until the required optical density is reached at which point an oxidase biocatalyst is induced using either a temperature shift or a chemical inducer such as rhamnose, arabinose or IPTG. Following induction the cells are recovered by centrifugation at 5,000×G for 30 minutes. The resulting cell pellet is stored at −20° C.

Preparation of the oxidase biocatalyst. Cells prepared by fermentation are lysed using a French Pressure Cell. The resulting lysate has 0.2% (w/v) polyethyleneimine added, and is centrifuged at 10,000×G for 30 minutes at 4° C. The pellet is discarded and the supernatant containing the oxidase biocatalyst is recovered and stored at −20° C.

### Example 9

### Oxidation of D-Tert-Leucine Using D-AAO Variant

Racemic tert-leucine (26.2 g, 0.1998 mole) is dissolved in water (350 ml) and adjusted to pH 7.5 with 1 M sodium hydroxide. The racemic tert-leucine solution is transferred to a temperature controlled jacketed reaction vessel at 30° C. and agitated at 200 rpm using an overhead stirrer. Oxygen gas is sparged into the reaction solution at 0.5 L/min. Micrococcus catalase (1 ml) and 50% polypropylene glycol (1 ml) is added. T. variabilis pPOT3/TvDAAO TBG40 (148 ml cell free extract solution prepared from 22.5 g of cells) is then charged to the vessel and the reaction is monitored by HPLC analysis. Chiral HPLC analysis indicated the reaction reached completion in 19 hr, (L-tert-leucine at >99% enantiomeric excess). The oxidase treated solution is then transferred to a 1 L round bottomed flask and concentrated sulfuric acid is added until pH 1.0. Celite (10 g) is added and the mixture is stirred to ensure good dispersion of the celite. The solids are filtered and the filtrate is collected for further processing.

Certain modifications and improvements will occur to those skilled in the art upon a reading of the foregoing description. The above-mentioned examples are provided to serve the purpose of clarifying the aspects of the invention and it will be apparent to one skilled in the art that they do not serve to limit the scope of the invention. While the specific embodiments have been illustrated and described, numerous modifications come to mind without significantly departing from the spirit of the invention, and the scope of protection is only limited by the scope of the accompanying claims.

What is claimed is:

1. A variant biocatalyst comprising:
a genetically mutated variant of a wild type enzyme wherein the wild type enzyme is a D-amino acid oxidase;
wherein the variant biocatalyst exhibits increased biocatalytic activity towards a D-amino acid substrate compared to the wild type enzyme.

2. The biocatalyst of claim 1, wherein the amino acid substrate comprises a branched chain amino acid, a halogenated amino acid, a straight chain amino acid, adamantly amino acid or a functionalized amino acid.

3. The biocatalyst of claim 1, wherein the amino acid substrate comprises D-tert-leucine, D-norvaline, D-2-aminobutyrate, D-alanine, D-isoleucine, D-valine, D-methionine, D-hydroxyadamantlyglycine, D-penicillamine, or D-norleucine.

4. The biocatalyst of claim 1, wherein the genetic mutation comprises at least one amino acid substitution in a region from Gly41 to Asp56 in the wild type D-amino acid oxidase of T. variabilis or in a substantially homologous D-amino acid oxidase.

5. The biocatalyst of claim 1, wherein the genetic mutation comprises at least one amino acid substitution in a region from Ile86 to Val118 in the wild type D-amino acid oxidase of T. variabilis or in a substantially homologous D-amino acid oxidase.

6. The biocatalyst of claim 1, wherein the genetic mutation comprises at least one amino acid substitution in a region from Ser228 to Met245 in the wild type D-amino acid oxidase of T. variabilis or in a substantially homologous D-amino acid oxidase.

7. The biocatalyst of claim 1, wherein the genetic mutation comprises at least one amino acid substitution in a region from Pro43 to Gln68 in the wild type D-amino acid oxidase of S. coelicolor or in a substantially homologous D-amino acid oxidase.

8. The biocatalyst of claim 1, wherein the genetic mutation comprises at least one amino acid substitution in a region from Leu93 to Leu113 in the wild type D-amino acid oxidase of S. coelicolor or in a substantially homologous D-amino acid oxidase.

9. The biocatalyst of claim 1, wherein the genetic mutation comprises at least one amino acid substitute at Val27, Gly101, His141 and/or Thr218, in the wild type D-amino acid oxidase of S. coelicolor or in a substantially homologous D-amino acid oxidase.

10. The biocatalyst of claim 1, wherein the genetic mutation comprises at least one amino acid substitute at Asp7, Glu8, Gly18, Val27, Ser49, Val50, Arg60, Glu62, Gln68, Leu93, Glu99, Thr100, Gly101, Asp106, Trp108, Ala111, Leu113, Leu129, His141, Asp158, Ala210, Ala211, Gly216, Thr218 and/or Phe221 in the wild type D-amino acid oxidase of S. coelicolor or in a substantially homologous D-amino acid oxidase.

11. The biocatalyst of claim 1, wherein the genetic mutation comprises at least one amino acid substitute at Tyr232 in the wild type D-amino acid oxidase of S. pombe or in a substantially homologous D-amino acid oxidase.

12. The biocatalyst of claim 1, wherein the genetic mutation comprises at least one amino acid substitute at Gly41, Tyr55, Asp56, Ala103, Ala106, Thr148, Met156, Met209, Ser228, Ala241, and/or Tyr243 in the wild type D-amino acid oxidase of T. variabilis or in a substantially homologous D-amino acid oxidase.

13. The biocatalyst of claim 1, wherein the wild type enzyme is a D-amino acid oxidase isolated from a microorganism comprising T. variabilis, S. coelicolor or S. pombe.

14. The biocatalyst of claim 1, wherein the variant biocatalyst expresses increased heat stability compared to the wild type enzyme.

15. A variant biocatalyst comprising:
a genetically mutated variant of a wild type enzyme wherein the wild type enzyme is a D-amino acid oxidase;
wherein the variant biocatalyst exhibits increased biocatalytic activity towards a D-amino acid substrate compared to the wild type enzyme, while retaining high enantioselectivity.

16. A method of preparing amino acids with high enantiomeric purity using a variant biocatalyst, comprising the steps of:
providing a racemic solution of amino acid substrates;
contacting the racemic solution with a genetically mutated biocatalyst variant of a wild type amino acid oxidase, wherein the biocatalyst variant exhibits increased biocatalytic activity and high enantioselectivity towards an amino acid substrate compared to the wild type enzyme;
oxidizing an enantiomer of the amino acid substrates with the variant biocatalyst to produce an imine or keto acid; and
simultaneously or sequentially reducing the imine and or keto acid in the presence of a non-selective reductant to yield a composition generally comprising one enantiomer of the amino acid substrate.

17. The method of claim 16, wherein the amino acid substrates comprise D-tert-leucine, D-norvaline, D-2-aminobutyrate, D-alanine, D-isoleucine, D-valine, D-methionine, D-hydroxyadamantlyglycine, D-penicillamine, or D-norleucine.

18. The biocatalyst of claim 16, wherein the genetic mutation comprises at least one amino acid substitute at Thr218, His141, Val27, Asp7, Glu62, Gln68, Val50, Leu93, Glu99, Ala211, Trp108, Gly18, Arg60, Gly216, Ser49, Ala210, Thr100, Asp158, Ala111, Leu129, Asp106, Glu8, Phe221, Leu113, Ala210 and/or Gly101 in the wild type D-amino acid oxidase of S. coelicolor or in a substantially homologous D-amino acid oxidase.

19. The biocatalyst of claim 16, wherein the genetic mutation comprises at least one amino acid substitute at Tyr232 in the wild type D-amino acid oxidase of S. pombe or in a substantially homologous D-amino acid oxidase.

20. The biocatalyst of claim 16, wherein the genetic mutation comprises at least one amino acid substitute at Tyr243, Ala241, Thr148, Met156, Met209, Gly41, Ala106, Tyr55, Asp56, Ala103 in the wild type D-amino acid oxidase of T. variabilis or in a substantially homologous D-amino acid oxidase.

21. The biocatalyst of claim 16, wherein the wild type enzyme is a D-amino acid oxidase isolated from a microorganism comprising T. variabilis, S. coelicolor, or S. pombe.


SEQUENCE LISTING (118 sequences)

SEQ ID NO: 1 - DNA, 984 bp, Artificial Sequence
  Description of Artificial Sequence Synthetic polynucleotide
gaattccatatggaaaccgagctggatgacgaacgcgatggtgaagtagttgtggttggcggtggggtgataggcctgaccactgccgttgtgctggcagaacgtgggcgtcgggtgcgcctgtggactcgtgaacccgcggaaagaaccacctccgtcgtggccggtgggctgtggtggccgtatcgcattgaaccagtagcactggcccaggcgtgggcgttgcgttcgctggatgtctatgaggaactggcagcccgtcctggtcagacgggcgttcgcatgttagaaggggtattgggagaaacgggcctggatgaagtggacggatgggcggcggcacggctaccgggactccgcgctgcatctgctgccgaatacgctggtacaggtttatgggcaagactgcctctgatcgatatgagcacccatttaccatggctgcgcgagcgactcctggctgcgggtggcaccgtcgaggaccgcgcggtcacagacctagcggaagccgatgcgccggtggtcgtaaactgcacaggccttggagcccgtgaactggttccggaccccgctgtcaggccggtcagaggccaactcgtggtagttgagaatccgggtattcacaattggcttgttgccgctgacgcggatagcggtgagactacgtactttttgcctcagccaggccgcttgttactggggggtacggcggaagaggatgcatggagtacagagcctgatccggaagttgctgccgcaatcgtgcgtcggtgcgcggctctccgtccagaaattgcaggagcccgtgttctggctcatcttgttggccttcgcccggcgcgcgatgccgtacgactggagcgtggtactttgccggatggccggcgcttagtgcataactatggtcacggcggggcgggcgtgaccgtggcatggggctgtgcgcaagaagccgcacgtcttgcctcataagtcgacggatcc

SEQ ID NO: 2 - DNA, 26 bp, Artificial Sequence
  Description of Artificial Sequence Synthetic primer
gcatgtactaaggaggatcccatatg

SEQ ID NO: 3 - DNA, 19 bp, Artificial Sequence
  Description of Artificial Sequence Synthetic primer
atgcggccgcgtcgactta

SEQ ID NO: 4 - DNA, 25 bp, Artificial Sequence
  Description of Artificial Sequence Synthetic primer
gaacaatgttgactattttacctct

SEQ ID NO: 5 - DNA, 26 bp, Artificial Sequence
  Description of Artificial Sequence Synthetic primer
ggatctttaattaattagatctgcat

SEQ ID NO: 6 - DNA, 963 bp, Artificial Sequence
  Description of Artificial Sequence Synthetic polynucleotide
atggaaaccgagctggatgacgaacgcgatggtgaagtagttgtggttggcggtggggtgataggcctgaccactgccgttgtgctggcagaacgtgggcgtcgggtgcgcctgtggactcgtgaacccgcggaaagaaccacctccgtcgtggccggtgggctgtggtggccgtatcgcattgaaccagtagcactggcccaggcgtgggcgttgcgttcgctggatgtctatgaggaactggcagcccgtcctggtcagacgggcgttcgcatgttagaaggggtattgggagaaacgggcctggatgaagtggacggatgggcggcggcacggctaccgggactccgcgctgcatctgctgccgaatacgctggtacaggtttatgggcaagactgcctctgatcgatatgagcacccatttaccatggctgcgcgagcgactcctggctgcgggtggcaccgtcgaggaccgcgcggtcacagacctagcggaagccgatgcgccggtggtcgtaaactgcacaggccttggagcccgtgaactggttccggaccccgctgtcaggccggtcagaggccaactcgtggtagttgagaatccgggtattcacaattggcttgttgccgctgacgcggatagcggtgagattacgtactttttgcctcagccaggccgcttgttactggggggtacggcggaagaggatgcatggagtacagagcctgatccggaagttgctgccgcaatcgtgcgtcggtgcgcggctctccgtccagaaattgcaggagcccgtgttctggctcatcttgttggccttcgcccggcgcgcgatgccgtacgactggagcgtggtactttgccggatggccggcgcttagtgcataactatggtcacggcggggcgggcgtgaccgtggcatggggctgtgcgcaagaagccgcacgtcttgcctcataa

SEQ ID NO: 7 - DNA, 963 bp, Artificial Sequence
  Description of Artificial Sequence Synthetic polynucleotide
atggaaaccgagctggatgacgaacgcgatggtgaagtagttgtggttggcggtggggtgataggcctgaccactgccgttgtgctggcagaacgtgggcgtcgggtgcgcctgtggactcgtgaacccgcggaaagaaccacctccgtcgtggccggtgggctgtggtggccgtatcgcattgaaccagtagcactggcccaggcgtgggcgttgcgttcgctggatgtctatgaggaactggcagcccgtcctggtcagacgggcgttcgcatgttagaaggggtattgggagaaacgggcctggatgaagtggacggatgggcggcggcacggctaccgggactccgcgctgcatctgctgccgaatacgctggtacaggtttatgggcaagactgcctctgatcgatatgagcacctatttaccatggctgcgcgagcgactcctggctgcgggtggcaccgtcgaggaccgcgcggtcacagacctagcggaagccgatgcgccggtggtcgtaaactgcacaggccttggagcccgtgaactggttccggaccccgctgtcaggccggtcagaggccaactcgtggtagttgagaatccgggtattcacaattggcttgttgccgctgacgcggatagcggtgagactacgtactttttgcctcagccaggccgcttgttactggggggtacggcggaagaggatgcatggagtacagagcctgatccggaagttgctgccgcaatcgtgcgtcggtgcgcggctctccgtccagaaattgcaggagcccgtgttctggctcatcttgttggccttcgcccggcgcgcgatgccgtacgactggagcgtggtactttgccggatggccggcgcttagtgcataactatggtcacggcggggcgggcgtgaccgtggcatggggctgtgcgcaagaagccgcacgtcttgcctcataa

SEQ ID NO: 8 - DNA, 963 bp, Artificial Sequence
  Description of Artificial Sequence Synthetic polynucleotide
atggaaaccgagctggatgacgaacgcgatggtgaagtagttgtggttggcggtggggtgataggcctgaccactgccattgtgctggcagaacgtgggcgtcgggtgcgcctgtggactcgtgaacccgcggaaagaaccacctccgtcgtggccggtgggctgtggtggccgtatcgcattgaaccagtagcactggcccaggcgtgggcgttgcgttcgctggatgtctatgaggaactggcagcccgtcctggtcagacgggcgttcgcatgttagaaggggtattgggagaaacgggcctggatgaagtggacggatgggcggcggcacggctaccgggactccgcgctgcatctgctgccgaatacgctggtacaggtttatgggcaagactgcctctgatcgatatgagcacccatttaccatggctgcgcgagcgactcctggctgcgggtggcaccgtcgaggaccgcgcggtcacagacctagcggaagccgatgcgccggtggtcgtaaactgcacaggccttggagcccgtgaactggttccggaccccgctgtcaggccggtcagaggccaactcgtggtagttgagaatccgggtattcacaattggcttgttgccgctgacgcggatagcggtgagactacgtactttttgcctcagccaggccgcttgttactggggggtacggcggaagaggatgcatggagtacagagcctgatccggaagttgctgccgcaatcgtgcgtcggtgcgcggctctccgtccagaaattgcaggagcccgtgttctggctcatcttgttggccttcgcccggcgcgcgatgccgtacgactggagcgtggtactttgccggatggccggcgcttagtgcataactatggtcacggcggggcgggcgtgaccgtggcatggggctgtgcgcaagaagccgcacgtcttgcctcataa

SEQ ID NO: 9 - DNA, 963 bp, Artificial Sequence; identical to SEQ ID NO: 7
  Description of Artificial Sequence Synthetic polynucleotide

SEQ ID NO: 10 - DNA, 39 bp, Artificial Sequence
  Description of Artificial Sequence Synthetic primer
ctgatcgatatgagcacctatttaccatggctgcgcgag

SEQ ID NO: 11 - DNA, 39 bp, Artificial Sequence
  Description of Artificial Sequence Synthetic primer
ctcgcgcagccatggtaaataggtgctcatatcgatcag

SEQ ID NO: 12 - DNA, 963 bp, Artificial Sequence
  Description of Artificial Sequence Synthetic polynucleotide
atggaaaccgagctggatgacgaacgcgatggtgaagtagttgtggttggcggtggggtgataggcctgaccactgccgttgtgctggcagaacgtgggcgtcgggtgcgcctgtggactcgtgaacccgcggaaagaaccacctccgtcgtggccggtgggctgtggtggccgtatcgcattgaaccagtagcactggcccaggcgtgggcgttgcgttcgctggatgtctatgaggaactggcagcccgtcctggtcagacgggcgttcgcatgttagaaggggtattgggagaaacgggcctggatgaagtggacggatgggcggcggcacggctaccgggactccgcgctgcatctgctgccgaatacgctggtacaggtttatgggcaagactgcctctgatcgatatgagcacctatttaccatggctgcgcgagcgactcctggctgcgggtggcaccgtcgaggaccgcgcggtcacagacctagcggaagccgatgcgccggtggtcgtaaactgcacaggccttggagcccgtgaactggttccggaccccgctgtcaggccggtcagaggccaactcgtggtagttgagaatccgggtattcacaattggcttgttgccgctgacgcggatagcggtgagattacgtactttttgcctcagccaggccgcttgttactggggggtacggcggaagaggatgcatggagtacagagcctgatccggaagttgctgccgcaatcgtgcgtcggtgcgcggctctccgtccagaaattgcaggagcccgtgttctggctcatcttgttggccttcgcccggcgcgcgatgccgtacgactggagcgtggtactttgccggatggccggcgcttagtgcataactatggtcacggcggggcgggcgtgaccgtggcatggggctgtgcgcaagaagccgcacgtcttgcctcataa

SEQ ID NO: 13 - DNA, 963 bp, Artificial Sequence
  Description of Artificial Sequence Synthetic polynucleotide
atggaaaccgagctggatgaagaacgcgatggtgaagtagttgtggttggcggtggggtgataggcctgaccactgccgttgtgctggcagaacgtgggcgtcgggtgcgcctgtggactcgtgaacccgcggaaagaaccacctccgtcgtggccggtgggctgtggtggccgtatcgcattgaaccagtagcactggcccaggcgtgggcgttgcgttcgctggatgtctatgaggaactggcagcccgtcctggtcagacgggcgttcgcatgttagaaggggtattgggagaaacgggcctggatgaagtggacggatgggcggcggcacggctaccgggactccgcgctgcatctgctgccgaatacgctggtacaggtttatgggcaagactgcctctgatcgatatgagcacctatttaccatggctgcgcgagcgactcctggctgcgggtggcaccgtcgaggaccgcgcggtcacagacctagcggaagccgatgcgccggtggtcgtaaactgcacaggccttggagcccgtgaactggttccggaccccgctgtcaggccggtcagaggccaactcgtggtagttgagaatccgggtattcacaattggcttgttgccgctgacgcggatagcggtgagattacgtactttttgcctcagccaggccgcttgttactggggggtacggcggaagaggatgcatggagtacagagcctgatccggaagttgctgccgcaatcgtgcgtcggtgcgcggctctccgtccagaaattgcaggagcccgtgttctggctcatcttgttggccttcgcccggcgcgcgatgccgtacgactggagcgtggtactttgccggatggccggcgcttagtgcataactatggtcacggcggggcgggcgtgaccgtggcatggggctgtgcgcaagaagccgcacgtcttgcctcataa

SEQ ID NO: 14 - DNA, 963 bp, Artificial Sequence
  Description of Artificial Sequence Synthetic polynucleotide
atggaaaccgagctggatgacgaacgcgatggtgaagtagttgtggttggcggtggggtgataggcctgaccactgccgttgtgctggcagaacgtgggcgtcgggtgcgcctgtggactcgtgaacccgcggaaagaaccacctccgtcgtggccggtgggctgtggtggccgtatcgcattaaaccagtagcactggcccaggcgtgggcgttgcgttcgctggatgtctatgaggaactggcagcccgtcctggtcagacgggcgttcgcatgttagaaggggtattgggagaaacgggcctggatgaagtggacggatgggcggcggcacggctaccgggactccgcgctgcatctgctgccgaatacgctggtacaggtttatgggcaagactgcctctgatcgatatgagcacctatttaccatggctgcgcgagcgactcctggctgcgggtggcaccgtcgaggaccgcgcggtcacagacctagcggaagccgatgcgccggtggtcgtaaactgcacaggccttggagcccgtgaactggttccggaccccgctgtcaggccggtcagaggccaactcgtggtagttgagaatccgggtattcacaattggcttgttgccgctgacgcggatagcggtgagattacgtactttttgcctcagccaggccgcttgttactggggggtacggcggaagaggatgcatggagtacagagcctgatccggaagttgctgccgcaatcgtgcgtcggtgcgcggctctccgtccagaaattgcaggagcccgtgttctggctcatcttgttggccttcgcccggcgcgcgatgccgtacgactggagcgtggtactttgccggatggccggcgcttagtgcataactatggtcacggcggggcgggcgtgaccgtggcatggggctgtgcgcaagaagccgcacgtcttgcctcataa

SEQ ID NO: 15 - DNA, 963 bp, Artificial Sequence
  Description of Artificial Sequence Synthetic polynucleotide
atggaaaccgagctggatgacgaacgcgatggtgaagtagttgtggttggcggtggggtgataggcctgaccactgccgttgtgctggcagaacgtgggcgtcgggtgcgcctgtggactcgtgaacccgcggaaagaaccacctccgtcgtggccggtgggctgtggtggccgtatcgcattgaaccagtagcactggcccgggcgtgggcgttgcgttcgctggatgtctatgaggaactggcagcccgtcctggtcagacgggcgttcgcatgttagaaggggtattgggagaaacgggcctggatgaagtggacggatgggcggcggcacggctaccgggactccgcgctgcatctgctgccgaatacgctggtacaggtttatgggcaagactgcctctgatcgatatgagcacctatttaccatggctgcgcgagcgactcctggctgcgggtggcaccgtcgaggaccgcgcggtcacagacctagcggaagccgatgcgccggtggtcgtaaactgcacaggccttggagcccgtgaactggttccggaccccgctgtcaggccggtcagaggccaactcgtggtagttgagaatccgggtattcacaattggcttgttgccgctgacgcggatagcggtgagattacgtactttttgcctcagccaggccgcttgttactggggggtacggcggaagaggatgcatggagtacagagcctgatccggaagttgctgccgcaatcgtgcgtcggtgcgcggctctccgtccagaaattgcaggagcccgtgttctggctcatcttgttggccttcgcccggcgcgcgatgccgtacgactggagcgtggtactttgccggatggccggcgcttagtgcataactatggtcacggcggggcgggcgtgaccgtggcatggggctgtgcgcaagaagccgcacgtcttgcctcataa

SEQ ID NO: 16 - DNA, 963 bp, Artificial Sequence
  Description of Artificial Sequence Synthetic polynucleotide
atggaaaccgagctggatgacgaacgcgatggtgaagtagttgtggttggcggtggggtgataggcctgaccactgccgttgtgctggcagaacgtgggcgtcgggtgcgcctgtggactcgtgaacccgcggaaagaaccacctccgccgtggccggtgggctgtggtggccgtatcgcattgaaccagtagcactggcccaggcgtgggcgttgcgttcgctggatgtctatgaggaactggcagcccgtcctggtcagacgggcgttcgcatgttagaaggggtattgggagaaacgggcctggatgaagtggacggatgggcggcggcacggctaccgggactccgcgctgcatctgctgccgaatacgctggtacaggtttatgggcaagactgcctctgatcgatatgagcacctatttaccatggctgcgcgagcgactcctggctgcgggtggcaccgtcgaggaccgcgcggtcacagacctagcggaagccgatgcgccggtggtcgtaaactgcacaggccttggagcccgtgaactggttccggaccccgctgtcaggccggtcagaggccaactcgtggtagttgagaatccgggtattcacaattggcttgttgccgctgacgcggatagcggtgagattacgtactttttgcctcagccaggccgcttgttactggggggtacggcggaagaggatgcatggagtacagagcctgatccggaagttgctgccgcaatcgtgcgtcggtgcgcggctctccgtccagaaattgcaggagcccgtgttctggctcatcttgttggccttcgcccggcgcgcgatgccgtacgactggagcgtggtactttgccggatggccggcgcttagtgcataactatggtcacggcggggcgggcgtgaccgtggcatggggctgtgcgcaagaagccgcacgtcttgcctcataa

SEQ ID NO: 17 - DNA, 963 bp, Artificial Sequence
  Description of Artificial Sequence Synthetic polynucleotide
atggaaaccgagctggatgacgaacgcgatggtgaagtagttgtggttggcggtggggtgataggcctgaccactgccgttgtgctggcagaacgtgggcgtcgggtgcgcctgtggactcgtgaacccgcggaaagaaccacctccgtcgtggccggtgggctgtggtggccgtatcgcattgaaccagtagcactggcccaggcgtgggcgttgcgttcgctggatgtctatgaggaactggcagcccgtcctggtcagacgggcgttcgcatggtagaaggggtattgggagaaacgggcctggatgaagtggacggatgggcggcggcacggctaccgggactccgcgctgcatctgctgccgaatacgctggtacaggtttatgggcaagactgcctctgatcgatatgagcacctatttaccatggctgcgcgagcgactcctggctgcgggtggcaccgtcgaggaccgcgcggtcacagacctagcggaagccgatgcgccggtggtcgtaaactgcacaggccttggagcccgtgaactggttccggaccccgctgtcaggccggtcagaggccaactcgtggtagttgagaatccgggtattcacaattggcttgttgccgctgacgcggatagcggtgagattacgtactttttgcctcagccaggccgcttgttactggggggtacggcggaagaggatgcatggagtacagagcctgatccggaagttgctgccgcaatcgtgcgtcggtgcgcggctctccgtccagaaattgcaggagcccgtgttctggctcatcttgttggccttcgcccggcgcgcgatgccgtacgactggagcgtggtactttgccggatggccggcgcttagtgcataactatggtcacggcggggcgggcgtgaccgtggcatggggctgtgcgcaagaagccgcacgtcttgcctcataa

SEQ ID NO: 18 - DNA, 963 bp, Artificial Sequence
  Description of Artificial Sequence Synthetic polynucleotide
atggaaaccgagctggatgacgaacgcgatggtgaagtagttgtggttggcggtggggtgataggcctgaccactgccgttgtgctggcagaacgtgggcgtcgggtgcgcctgtggactcgtgaacccgcggaaagaaccacctccgtcgtggccggtgggctgtggtggccgtatcgcattgaaccagtagcactggcccaggcgtgggcgttgcgttcgctggatgtctatgaggaactggcagcccgtcctggtcagacgggcgttcgcatgttagaaggggtattgggaggaacgggcctggatgaagtggacggatgggcggcggcacggctaccgggactccgcgctgcatctgctgccgaatacgctggtacaggtttatgggcaagactgcctctgatcgatatgagcacctatttaccatggctgcgcgagcgactcctggctgcgggtggcaccgtcgaggaccgcgcggtcacagacctagcggaagccgatgcgccggtggtcgtaaactgcacaggccttggagcccgtgaactggttccggaccccgctgtcaggccggtcagaggccaactcgtggtagttgagaatccgggtattcacaattggcttgttgccgctgacgcggatagcggtgagattacgtactttttgcctcagccaggccgcttgttactggggggtacggcggaagaggatgcatggagtacagagcctgatccggaagttgctgccgcaatcgtgcgtcggtgcgcggctctccgtccagaaattgcaggagcccgtgttctggctcatcttgttggccttcgcccggcgcgcgatgccgtacgactggagcgtggtactttgccggatggccggcgcttagtgcataactatggtcacggcggggcgggcgtgaccgtggcatggggctgtgcgcaagaagccgcacgtcttgcctcataa

SEQ ID NO: 19 - DNA, 963 bp, Artificial Sequence
  Description of Artificial Sequence Synthetic polynucleotide
atggaaaccgagctggatgacgaacgcgatggtgaagtagttgtggttggcggtggggtgataggcctgaccactgccgttgtgctggcagaacgtgggcgtcgggtgcgcctgtggactcgtgaacccgcggaaagaaccacctccgtcgtggccggtgggctgtggtggccgtatcgcattgaaccagtagcactggcccaggcgtgggcgttgcgttcgctggatgtctatgaggaactggcagcccgtcctggtcagacgggcgttcgcatgttagaaggggtattgggagaaacgggcctggatgaagtggacggatgggcggcggcacggctaccgggactccgcgctgcatctgctgccgaatacgctggtacaggtttatgggcaagactgcctctgatcgatatgagcacctatttaccatggctgcgcgagcgactcctggctgcgggtggcaccgtcgaggaccgcgcggtcacagacctagcggaagccgatgcgccggtggtcgtaaactgcacaggccttggagcccgtgaactggttccggaccccgctgtcaggccggtcagaggccaactcgtggtagttgagaatccgggtattcacaattggcttgttgccactgacgcggatagcggtgagattacgtactttttgcctcagccaggccgcttgttactggggggtacggcggaagaggatgcatggagtacagagcctgatccggaagttgctgccgcaatcgtgcgtcggtgcgcggctctccgtccagaaattgcaggagcccgtgttctggctcatcttgttggccttcgcccggcgcgcgatgccgtacgactggagcgtggtactttgccggatggccggcgcttagtgcataactatggtcacggcggggcgggcgtgaccgtggcatggggctgtgcgcaagaagccgcacgtcttgcctcataa

SEQ ID NO: 20 - DNA, 963 bp, Artificial Sequence
  Description of Artificial Sequence Synthetic polynucleotide
atggaaaccgagctggatgacgaacgcgatggtgaagtagttgtggttggcggtggggtgataggcctgaccactgccgttgtgctggcagaacgtgggcgtcgggtgcgcctgtggactcgtgaacccgcggaaagaaccacctccgtcgtggccggtgggctgtggtggccgtatcgcattgaaccagtagcactggcccaggcgtgggcgttgcgttcgctggatgtctatgaggaactggcagcccgtcctggtcagacgggcgttcgcatgttagaaggggtattgggagaaacgggcctggatgaagtggacggacgggcggcggcacggctaccgggactccgcgctgcatctgctgccgaatacgctggtacaggtttatgggcaagactgcctctgatcgatatgagcacctatttaccatggctgcgcgagcgactcctggctgcgggtggcaccgtcgaggaccgcgcggtcacagacctagcggaagccgatgcgccggtggtcgtaaactgcacaggccttggagcccgtgaactggttccggaccccgctgtcaggccggtcagaggccaactcgtggtagttgagaatccgggtattcacaattggcttgttgccgctgacgcggatagcggtgagattacgtactttttgcctcagccaggccgcttgttactggggggtacggcggaagaggatgcatggagtacagagcctgatccggaagttgctgccgcaatcgtgcgtcggtgcgcggctctccgtccagaaattgcaggagcccgtgttctggctcatcttgttggccttcgcccggcgcgcgatgccgtacgactggagcgtggtactttgccggatggccggcgcttagtgcataactatggtcacggcggggcgggcgtgaccgtggcatggggctgtgcgcaagaagccgcacgtcttgcctcataa

SEQ ID NO: 21 - DNA, 963 bp, Artificial Sequence
  Description of Artificial Sequence Synthetic polynucleotide
atggaaaccgagctggatgacgaacgcgatggtgaagtagttgtggttggcagtggggtgataggcctgaccactgccgttgtgctggcagaacgtgggcgtcgggtgcgcctgtggactcgtgaacccgcggaaagaaccacctccgccgtggccggtgggctgtggtggccgtatcgcattgaaccagtagcactggcccaggcgtgggcgttgcgttcgctggatgtctatgaggaactggcagcccgtcctggtcagacgggcgttcgcatgttagaaggggtattgggagaaacgggcctggatgaagtggacggatgggcggcggcacggctaccgggactccgcgctgcatctgctgccgaatacgctggtacaggtttatgggcaagactgcctctgatcgatatgagcacctatttaccatggctgcgcgagcgactcctggctgcgggtggcaccgtcgaggaccgcgcggtcacagacctagcggaagccgatgcgccggtggtcgtaaactgcacaggccttggagcccgtgaactggttccggaccccgctgtcaggccggtcagaggccaactcgtggtagttgagaatccgggtattcacaattggcttgttgccgctgacgcggatagcggtgagattacgtactttttgcctcagccaggccgcttgttactggggggtacggcggaagaggatgcatggagtacagagcctgatccggaagttgctgccgcaatcgtgcgtcggtgcgcggctctccgtccagaaattgcaggagcccgtgttctggctcatcttgttggccttcgcccggcgcgcgatgccgtacgactggagcgtggtactttgccggatggccggcgcttagtgcataactatggtcacggcggggcgggcgtgaccgtggcatggggctgtgcgcaagaagccgcacgtcttgcctcataa

SEQ ID NO: 22 - DNA, 963 bp, Artificial Sequence
  Description of Artificial Sequence Synthetic polynucleotide
atggaaaccgagctggatgacgaacgcgatggtgaagtagttgtggttggcggtggggtgataggcctgaccactgccgttgtgctggcagaacgtgggcgtcgggtgcgcctgtggactcgtgaacccgcggaaagaaccacctccgccgtggccggtgggctgtggtggccgtatctcattgaaccagtagcactggcccaggcgtgggcgttgcgttcgctggatgtctatgaggaactggcagcccgtcctggtcagacgggcgttcgcatgttagaaggggtattgggagaaacgggcctggatgaagtggacggatgggcggcggcacggctaccgggactccgcgctgcatctgctgccgaatacgctggtacaggtttatgggcaagactgcctctgatcgatatgagcacctatttaccatggctgcgcgagcgactcctggctgcgggtggcaccgtcgaggaccgcgcggtcacagacctagcggaagccgatgcgccggtggtcgtaaactgcacaggccttggagcccgtgaactggttccggaccccgctgtcaggccggtcagaggccaactcgtggtagttgagaatccgggtattcacaattggcttgttgccgctgacgcggatagcggtgagattacgtactttttgcctcagccaggccgcttgttactggggggtacggcggaagaggatgcatggagtacagagcctgatccggaagttgctgccgcaatcgtgcgtcggtgcgcggctctccgtccagaaattgcaggagcccgtgttctggctcatcttgttggccttcgcccggcgcgcgatgccgtacgactggagcgtggtactttgccggatggccggcgcttagtgcataactatggtcacggcggggcgggcgtgaccgtggcatggggctgtgcgcaagaagccgcacgtcttgcctcataa

SEQ ID NO: 23 - DNA, 963 bp, Artificial Sequence
  Description of Artificial Sequence Synthetic polynucleotide
atggaaaccgagctggatgacgaacgcgatggtgaagtagttgtggttggcggtggggtgataggcctgaccactgccgttgtgctggcagaacgtgggcgtcgggtgcgcctgtggactcgtgaacccgcggaaagaaccacctccgccgtggccggtgggctgtggtggccgtatcgcattgaaccagtagcactggcccaggcgtgggcgttgcgttcgctggatgtctatgaggaactggcagcccgtcctggtcagacgggcgttcgcatgttagaaggggtattgggagaaacgggcctggatgaagtggacggatgggcggcggcacggctaccgggactccgcgctgcatctgctgccgaatacgctggtacaggtttatgggcaagactgcctctgatcgatatgagcacctatttaccatggctgcgcgagcgactcctggctgcgggtggcaccgtcgaggaccgcgcggtcacagacctagcggaagccgatgcgccggtggtcgtaaactgcacaggccttggagcccgtgaactggttccggaccccgctgtcaggccggtcagaggccaactcgtggtagttgagaatccgggtattcacaattggcttgttgccgctgacgcggatagcagtgagattacgtactttttgcctcagccaggccgcttgttactggggggtacggcggaagaggatgcatggagtacagagcctgatccggaagttgctgccgcaatcgtgcgtcggtgcgcggctctccgtccagaaattgcaggagcccgtgttctggctcatcttgttggccttcgcccggcgcgcgatgccgtacgactggagcgtggtactttgccggatggccggcgcttagtgcataactatggtcacggcggggcgggcgtgaccgtggcatggggctgtgcgcaagaagccgcacgtcttgcctcataa

SEQ ID NO: 24 - DNA, 963 bp, Artificial Sequence
  Description of Artificial Sequence Synthetic polynucleotide
atggaaaccgagctggatgacgaacgcgatggtgaagtagttgtggttggcggtggggtgataggcctgaccactgccgttgtgctggcagaacgtgggcgtcgggtgcgcctgtggactcgtgaacccgcggaaagaaccaccgccgccgtggccggtgggctgtggtggccgtatcgcattgaaccagtagcactggcccaggcgtgggcgttgcgttcgctggatgtctatgaggaactggcagcccgtcctggtcagacgggcgttcgcatgttagaaggggtattgggagaaacgggcctggatgaagtggacggatgggcggcggcacggctaccgggactccgcgctgcatctgctgccgaatacgctggtacaggtttatgggcaagactgcctctgatcgatatgagcacctatttaccatggctgcgcgagcgactcctggctgcgggtggcaccgtcgaggaccgcgcggtcacagacctagcggaagccgatgcgccggtggtcgtaaactgcacaggccttggagcccgtgaactggttccggaccccgctgtcaggccggtcagaggccaactcgtggtagttgagaatccgggtattcacaattggcttgttaccgctgacgcggatagcggtgagattacgtactttttgcctcagccaggccgcttgttactggggggtacggcggaagaggatgcatggagtacagagcctgatccggaagttgctgccgcaatcgtgcgtcggtgcgcggctctccgtccagaaattgcaggagcccgtgttctggctcatcttgttggccttcgcccggcgcgcgatgccgtacgactggagcgtggtactttgccggatggccggcgcttagtgcataactatggtcacggcggggcgggcgtgaccgtggcatggggctgtgcgcaagaagccgcacgtcttgcctcataa

SEQ ID NO: 25 - DNA, 963 bp, Artificial Sequence
  Description of Artificial Sequence Synthetic polynucleotide
atggaaaccgagctggatgacgaacgcgatggtgaagtagttgtggttggcggtggggtgataggcctgaccactgccgttgtgctggcagaacgtgggcgtcgggtgcgcctgtggactcgtgaacccgcggaaagaaccacctccgccgtggccggtgggctgtggtggccgtatcgcattgaaccagtagcactggcccaggcgtgggcgttgcgttcgctggatgtctatgaggaactggcagcccgtcctggtcagacgggcgttcgcatgttagaaggggtattgggagaaatgggcctggatgaagtggacggatgggcggcggcacggctaccgggactccgcgctgcatctgctgccgaatacgctggtacaggtttatgggcaagactgcctctgatcgatatgagcacctatttaccatggctgcgcgagcgactcctggctgcgggtggcaccgtcgaggaccgcgcggtcacagacctagcggaagccgatgcgccggtggtcgtaaactgcacaggccttggagcccgtgaactggttccggaccccgctgtcaggccggtcagaggccaactcgtggtagttgagaatccgggtattcacaattggcttgttaccgctgacgcggatagcggtgagattacgtactttttgcctcagccaggccgcttgttactggggggtacggcggaagaggatgcatggagtacagagcctgatccggaagttgctgccgcaatcgtgcgtcggtgcgcggctctccgtccagaaattgcaggagcccgtgttctggctcatcttgttggccttcgcccggcgcgcgatgccgtacgactggagcgtggtactttgccggatggccggcgcttagtgcataactatggtcacggcggggcgggcgtgaccgtggcatggggctgtgcgcaagaagccgcacgtcttgcctcataa

SEQ ID NO: 26 - DNA, 963 bp, Artificial Sequence
  Description of Artificial Sequence Synthetic polynucleotide
atggaaaccgagctggatgacgaacgcgatggtgaagtagttgtggttggcggtggggtgataggcctgaccactgccgttgtgctggcagaacgtgggcgtcgggtgcgcctgtggactcgtgaacccgcggaaagaaccacctccgccgtggccggtgggctgtggtggccgtatcgcattgaaccagtagcactggcccaggcgtgggcgttgcgttcgctggatgtctatgaggaactggcagcccgtcctggtcagacgggcgttcgcatgttagaaggggtattgggagaaacgggcctggatgaagtggacggatgggcggcggcacggctaccgggactccgcgctgcatctgctgccgaatacgctggtacaggtttatgggcaagactgcctctgatcgatatgagcacctatttaccatggctgcgcgagcgactcctggctgcgggtggcaccgtcgaggaccgcgcggtcacagacctagcggaagccgatgcgccggtggtcgtaaactgcacaggccttggagcccgtgaactggttccggaccccgctgtcaggccggtcagaggccaactcgtggtagttgagaatccgggtattcacaattggcttgttgccactgacgcggatagcggtgagattacgtactttttgcctcagccaggccgcttgttactggggggtacggcggaagaggatgcatggagtacagagcctgatccggaagttgctgccgcaatcgtgcgtcggtgcgcggctctccgtccagaaattgcaggagcccgtgttctggctcatcttgttggccttcgcccggcgcgcgatgccgtacgactggagcgtggtactttgccggatggccggcgcttagtgcataactatggtcacggcggggcgggcgtgaccgtggcatggggctgtgcgcaagaagccgcacgtcttgcctcataa

SEQ ID NO: 27 - DNA, 963 bp, Artificial Sequence
  Description of Artificial Sequence Synthetic polynucleotide
atggaaaccgagctggatgacgaacgcgatggtgaagtagttgtggttggcggtggggtgataggcctgaccactgccgttgtgctggcagaacgtgggcgtcgggtgcgcctgtggactcgtgaacccgcggaaagaaccacctccgccgtggccggtgggctgtggtggccgtatcgcattgaaccagtagcactggcccaggcgtgggcgttgcgttcgctggatgtctatgaggaactggcagcccgtcctggtcagacgggcgttcgcatggtagaaggggtattgggagaaacgggcctggatgaagtggacggatgggcggcggcacggctaccgggactccgcgctgcatctgctgccgaatacgctggtacaggtttatgggcaagactgcctctgatcgatatgagcacctatttaccatggctgcgcgagcgactcctggctgcgggtggcaccgtcgaggaccgcgcggtcacagacctagcggaagccgatgcgccggtggtcgtaaactgcacaggccttggagcccgtgaactggttccggaccccgctgtcaggccggtcagaggccaactcgtggtagttgagaatccgggtattcacaattggcttgttgccgctgacgcggatagcggtgagattacgtactttttgcctcagccaggccgcttgttactggggggtacggcggaagaggatgcatggagtacagagcctgatccggaagttgctgccgcaatcgtgcgtcggtgcgcggctctccgtccagaaattgcaggagcccgtgttctggctcatcttgttggccttcgcccggcgcgcgatgccgtacgactggagcgtggtactttgccggatggccggcgcttagtgcataactatggtcacggcggggcgggcgtgaccgtggcatggggctgtgcgcaagaagccgcacgtcttgcctcataa

SEQ ID NO: 28 - DNA, 963 bp, Artificial Sequence
  Description of Artificial Sequence Synthetic polynucleotide
atggaaaccgagctggatgacgaacgcgatggtgaagtagttgtggttggcggtggggtgataggcctgaccactgccgttgtgctggcagaacgtgggcgtcgggtgcgcctgtggactcgtgaacccgcggaaagaaccacctccgccgtggccggtgggctgtggtggccgtatcgcattgaaccagtagcactggcccaggcgtgggcgttgcgttcgctggatgtctatgaggaactggcagcccgtcctggtcagacgggcgttcgcatgttagaaggggtattgggagaaacgggcctggatgaagtggacggatgggcggcggcacggctaccgggactccgcgctgcatctgctgccgaatacgctggtacaggtttatgggcaagactgcctctgatcgatatgagcacctatttaccatggctgcgcgagcgactcctggctgcgggtggcaccgtcgagtaccgcgcggtcacagacctagcggaagccgatgcgccggtggtcgtaaactgcacaggccttggagcccgtgaactggttccggaccccgctgtcaggccggtcagaggccaactcgtggtagttgagaatccgggtattcacaattggcttgttgccgctgacgcggatagcggtgagattacgtactttttgcctcagccaggccgcttgttactggggggtacggcggaagaggatgcatggagtacagagcctgatccggaagttgctgccgcaatcgtgcgtcggtgcgcggctctccgtccagaaattgcaggagcccgtgttctggctcatcttgttggccttcgcccggcgcgcgatgccgtacgactggagcgtggtactttgccggatggccggcgcttagtgcataactatggtcacggcggggcgggcgtgaccgtggcatggggctgtgcgcaagaagccgcacgtcttgcctcataa

SEQ ID NO: 29 - DNA, 963 bp, Artificial Sequence
  Description of Artificial Sequence Synthetic polynucleotide
atggaaaccgagctggatgacgaacgcgatggtgaagtagttgtggttggcggtggggtgataggcctgaccactgccgttgtgctggcagaacgtgggcgtcgggtgcgcctgtggactcgtgaacccgcggaaagaaccacctccgccgtggccggtgggctgtggtggccgtatcgcattgaaccagtagcactggcccaggcgtgggcgttgcgttcgctggatgtctatgaggaactggcagcccgtcctggtcagacgggcgttcgcatgttagaaggggtattgggagaaacgggcctggatgaagtggacggatgggcggcgggacggctaccgggactccgcgctgcatctgctgccgaatacgctggtacaggtttatgggcaagactgcctctgatcgatatgagcacctatttaccatggctgcgcgagcgactcctggctgcgggtggcaccgtcgaggaccgcgcggtcacagacctagcggaagccgatgcgccggtggtcgtaaactgcacaggccttggagcccgtgaactggttccggaccccgctgtcaggccggtcagaggccaactcgtggtagttgagaatccgggtattcacaattggcttgttgccgctgacgcggatagcggtgagattacgtactttttgcctcagccaggccgcttgttactggggggtacggcggaagaggatgcatggagtacagagcctgatccggaagttgctgccgcaatcgtgcgtcggtgcgcggctctccgtccagaaattgcaggagcccgtgttctggctcatcttgttggccttcgcccggcgcgcgatgccgtacgactggagcgtggtactttgccggatggccggcgcttagtgcataactatggtcacggcggggcgggcgtgaccgtggcatggggctgtgcgcaagaagccgcacgtcttgcctcataa

SEQ ID NO: 30 - DNA, 963 bp, Artificial Sequence
  Description of Artificial Sequence Synthetic polynucleotide
atggaaaccgagctggatgacgaacgcgatggtgaagtagttgtggttggcggtggggtgataggcctgaccactgccgttgtgctggcagaacgtgggcgtcgggtgcgcctgtggactcgtgaacccgcggaaagaaccacctccgccgtggccggtgggctgtggtggccgtatcgcattgaaccagtagcactggcccaggcgtgggcgttgcgttcgctggatgtctatgaggaactggcagcccgtcctggtcagacgggcgttcgcatgttagaaggggtattgggagaaacgggcctggatgaagtggacggatgggcggcggcacggctaccgggactccgcgctgcatctgctgccgaatacgctggtacaggtgtatgggcaagactgcctctgatcgatatgagcacctatttaccatggctgcgcgagcgactcctggctgcgggtggcaccgtcgaggaccgcgcggtcacagacctagcggaagccgatgcgccggtggtcgtaaactgcacaggccttggagcccgtgaactggttccggaccccgctgtcaggccggtcagaggccaactcgtggtagttgagaatccgggtattcacaattggcttgttgccgctgacgcggatagcggtgagattacgtactttttgcctcagccaggccgcttgttactggggggtacggcggaagaggatgcatggagtacagagcctgatccggaagttgctgccgcaatcgtgcgtcggtgcgcggctctccgtccagaaattgcaggagcccgtgttctggctcatcttgttggccttcgcccggcgcgcgatgccgtacgactggagcgtggtactttgccggatggccggcgcttagtgcataactatggtcacggcggggcgggcgtgaccgtggcatggggctgtgcgcaagaagccgcacgtcttgcctcataa

SEQ ID NO: 31 - DNA, 963 bp, Artificial Sequence
  Description of Artificial Sequence Synthetic polynucleotide
atggaaaccgagctggatgacgaacgcgatggtgaagtagttgtggttggcggtggggtgataggcctgaccactgccgttgtgctggcagaacgtgggcgtcgggtgcgcctgtggactcgtgaacccgcggaaagaaccacctccgccgtggccggtgggctgtggtggccgtatcgcattgaaccagtagcactggcccaggcgtgggcgttgcgttcgctggatgtctatgaggaactggcagcccgtcctggtcagacgggcgttcgcatgttagaaggggtattgggagaaacgagcctggatgaagtggtcggatgggcggcggcacggctaccgggactccgcgctgcatctgctgccgaatacgctggtacaggtttatgggcaagactgcctctgatcgatatgagcacctatttaccatggctgcgcgagcgactcctggctgcgggtggcaccgtcgaggaccgcgcggtcacagacctagcggaagccgatgcgccggtggtcgtaaactgcacaggccttggagcccgtgaactggttccggaccccgctgtcaggccggtcagaggccaactcgtggtagttgagaatccgggtattcacaattggcttgttgccgctgacgcggatagcggtgagattacgtactttttgcctcagccaggccgcttgttactggggggtacggcggaagaggatgcatggagtacagagcctgatccggaagttgctgccgcaatcgtgcgtcggtgcgcggctctccgtccagaaattgcaggagcccgtgttctggctcatcttgttggccttcgcccggcgcgcgatgccgtacgactggagcgtggtactttgccggatggccggcgcttagtgcataactatggtcacggcggggcgggcgtgaccgtggcatggggctgtgcgcaagaagccgcacgtcttgcctcataa

SEQ ID NO: 32 - DNA, 963 bp, Artificial Sequence
  Description of Artificial Sequence Synthetic polynucleotide
atggaaaccgagctggatgacgtacgcgatggtgaagtagttgtggttggcagtggggtgataggcctgaccactgccgttgtgctggcagaacgtgggcgtcgggtgcgcctgtggactcgtgaacccgcggaaagaaccacctccgccgtggccggtgggctgtggtggccgtatcgcattgaaccagtagcactggcccaggcgtgggcgttgcgttcgctggatgtctatgaggaactggcagcccgtcctggtcagacgggcgttcgcatgttagaaggggtattgggagaaacgggcctggatgaagtggacggatgggcggcggcacggctaccgggactccgcgctgcatctgctgccgaatacgctggtacaggtttatgggcaagactgcctctgatcgatatgagcacctatttaccatggctgcgcgagcgactcctggctgcgggtggcaccgtcgaggaccgcgcggtcacagacctagcggaagccgatgcgccggtggtcgtaaactgcacaggccttggagcccgtgaactggttccggaccccgctgtcaggccggtcagaggccaactcgtggtagttgagaatccgggtattcacaattggcttgttgccgctgacgcggatagcggtgagattacgtactttttgcctcagccaggccgcttgttactggggggtacggcggaagaggatgcatggagtacagagcctgatccggaagttgctgccgcaatcgtgcgtcggtgcgcggctctccgtccagaaattgcaggagcccgtgttctggctcatcttgttggccttcgcccggcgcgcgatgccgtacgactggagcgtggtactttgccggatggccggcgcttagtgcataactatggtcacggcggggcgggcgtgaccgtggcatggggctgtgcgcaagaagccgcacgtcttgcctcataa

SEQ ID NO: 33 - DNA, 963 bp, Artificial Sequence
  Description of Artificial Sequence Synthetic polynucleotide
atggaaaccgagctggatgacgaacgcgatggtgaagtagttgtggttggcggtggggtgataggcctgaccactgccgttgtgctggcagaacgtgggcgtcgggtgcgcctgtggactcgtgaacccgcggaaagaaccacctccgccgtggccggtgggctgtggtggccgtatcgcattgaaccagtagcactggcccaggcgtgggcgttgcgttcgctggatgtctatgaggaactggcagcccgtcctggtcagacgggcgttcgcatgttagaaggggtattgggagaaacgggcctggatgaagtggacggatgggcggcggcacggctaccgggactccgcgctgcatctgctgccgaatacgctggtacaggtttatgggcaagactgcctctgatcgatatgagcacctatttaccatggctgcgcgagcgactcctggctgcgggtggcaccgtcgaggaccgcgcggtcacagacctagcggaagccgatgcgccggtggtcgtaaactgcacaggccttggagcccgtgaactggttccggaccccgctgtcaggccggtcagaggccaactcgtggtagttgagaatccgggtattcacaattggcttgttgccgctgacgcggatagcggtgagattacgtactatttgcctcagccaggccgcttgttactggggggtacggcggaagaggatgcatggagtacagagcctgatccggaagttgctgccgcaatcgtgcgtcggtgcgcggctctccgtccagaaattgcaggagcccgtgttctggctcatcttgttggccttcgcccggcgcgcgatgccgtacgactggagcgtggtactttgccggatggccggcgcttagtgcataactatggtcacggcggggcgggcgtgaccgtggcatggggctgtgcgcaagaagccgcacgtcttgcctcataa

SEQ ID NO: 34 - DNA, 963 bp, Artificial Sequence
  Description of Artificial Sequence Synthetic polynucleotide
atggaaaccgagctggatgacgaacgcgatggtgaagtagttgtggttggcggtggggtgataggcctgaccactgccgttgtgctggcagaacgtgggcgtcgggtgcgcctgtggactcgtgaacccgcggaaagaaccacctccgccgtggccggtgggctgtggtggccgtatcgcattgaaccagtagcactggcccaggcgtgggcgttgcgttcgctggatgtctatgaggaactggcagcccgtcctggtcagacgggcgttcgcatgttagaaggggtattgggagaaacgagcctggatgaagtggacggatgggcggcggcacggataccgggactccgcgctgcatctgctgccgaatacgctggtacaggtttatgggcaagactgcctctgatcgatatgagcacctatttaccatggctgcgcgagcgactcctggctgcgggtggcaccgtcgaggaccgcgcggtcacagacctagcggaagccgatgcgccggtggtcgtaaactgcacaggccttggagcccgtgaactggttccggaccccgctgtcaggccggtcagaggccaactcgtggtagttgagaatccgggtattcacaattggcttgttgccgctgacgcggatagcggtgagattacgtactttttgcctcagccaggccgcttgttactggggggtacggcggaagaggatgcatggagtacagagcctgatccggaagttgctgccgcaatcgtgcgtcggtgcgcggctctccgtccagaaattgcaggagcccgtgttctggctcatcttgttggccttcgcccggcgcgcgatgccgtacgactggagcgtggtactttgccggatggccggcgcttagtgcataactatggtcacggcggggcgggcgtgaccgtggcatggggctgtgcgcaagaagccgcacgtcttgcctcataa

SEQ ID NO: 35 - DNA, 963 bp, Artificial Sequence
  Description of Artificial Sequence Synthetic polynucleotide
atggaaaccgagctggatgacgaacgcgatggtgaagtagttgtggttggcggtggggtgataggcctgaccactgccgttgtgctggcagaacgtgggcgtcgggtgcgcctgtggactcgtgaacccgcggaaagaaccacctccgccgtggccggtgggctgtggtggccgtatcgcattgaaccagtagcactggcccaggcgtgggcgttgcgttcgctggatgtctatgaggaactggcagcccgtcctggtcagacgggcgttcgcatgttagaaggggtattgggagaaacgggcctggatgaagtggacggatgggcggcggcacggctaccgggactccgcgctgcatctgctgccgaatacgctggtacaggtttatgggcaagactgcctctgatcgatatgagcacctatttaccatggctgcgcgagcgactcctggctgcgggtggcaccgtcgaggaccgcgcggtcacagacctagcggaagccgatgcgccggtggtcgtaaactgcacaggccttggagcccgtgaactggttccggaccccgctgtcaggccggtcagaggccaactcgtggtagttgagaatccgggtattcacaattggcttgttaccgctgacgcggatagcggtgagattacgtactttttgcctcagccaggccgcttgttactggggggtacggcggaagaggatgcatggagtacagagcctgatccggaagttgctgccgcaatcgtgcgtcggtgcgcggctctccgtccagaaattgcaggagcccgtgttctggctcatcttgttggccttcgcccggcgcgcgatgccgtacgactggagcgtggtactttgccggatggccggcgcttagtgcataactatggtcacggcggggcgggcgtgaccgtggcatggggctgtgcgcaagaagccgcacgtcttgcctcataa

SEQ ID NO: 36 - DNA, 1068 bp, Artificial Sequence
  Description of Artificial Sequence Synthetic polynucleotide
gaattccatatgactaaggagaacaagcctcgcgacattgtcatcgtgggggcgggtgttattggtctgacgactgcctggattctgagcgatctgggcctggcgccacgaataaaagtcattgcgaaatatacgcctgaggatcgctctgttgagtacacgtcaccttgggctggggcgaatttttgcagtataagtgcaactgatgacaacgctttacggtgggacaagattacgtaccaccgttttgcgtatctcgcgaaaaccagaccggaagccggtattcgctttgcggatctccgcgagttatgggagtacgagccgaaacatgataaaattcgttcctggaatacttacgtgcgcgacttcaaagtgatcccggaaaaggacctgccgggtgagtgcatctatggccataaggcaaccaccttccttatcaacgccccgcattatctgaattatatgtataaactgttgatagaagcaggtgtagaatttgaaaaaaaggaactgtctcacattaaagagaccgtcgaagagacaccagaagcgagtgtcgtgtttaattgtacgggcctgtgggcctcaaaattgggcggagtggaagatccggatgtgtacccgacacggggacatgtggttctagttaaagctccgcatgttaccgaaacccgtatcttgaacggcaaaaattcggatacctatatcattcccaggccgctgaatgggggcgttatctgtgggggctttatgcagcccggtaactgggatcgtgaaattcacccagaagacacactggatattttaaaacgtacctctgctttgatgccagaactgttccatgggaaaggcccggaaggcgcagaaatcattcaggaatgcgtgggattccgccccagccgtaaaggtggcgccagagttgaactggatgtagtacctggtacatccgtcccgcttgtacacgactatggcgcttcgggaacgggttaccaagccggttatggtatggcacttgatagcgtgatgctcgcattaccaaaaatcaaacttgcctaagtcgacggatcc

SEQ ID NO: 37 - DNA, 39 bp, Artificial Sequence
  Description of Artificial Sequence Synthetic primer
ggcaaaaattcggataccnnkatcattcccaggccgctg

SEQ ID NO: 38 - DNA, 39 bp, Artificial Sequence
  Description of Artificial Sequence Synthetic primer
cagcggcctgggaatgatmnnggtatccgaatttttgcc

SEQ ID NO: 39 - DNA, 1047 bp, Artificial Sequence
  Description of Artificial Sequence Synthetic polynucleotide
atgactaaggagaacaagcctcgcgacattgtcatcgtgggggcgggtgttattggtctgacgactgcctggattctgagcgatctgggcctggcgccacgaataaaagtcattgcgaaatatacgcctgaggatcgctctgttgagtacacgtcaccttgggctggggcgaatttttgcagtataagtgcaactgatgacaacgctttacggtgggacaagattacgtaccaccgttttgcgtatctcgcgaaaaccagaccggaagccggtattcgctttgcggatctccgcgagttatgggagtacgagccgaaacatgataaaattcgttcctggaatacttacgtgcgcgacttcaaagtgatcccggaaaaggacctgccgggtgagtgcatctatggccataaggcaaccaccttccttatcaacgccccgcattatctgaattatatgtataaactgttgatagaagcaggtgtagaatttgaaaaaaaggaactgtctcacattaaagagaccgtcgaagagacaccagaagcgagtgtcgtgtttaattgtacgggcctgtgggcctcaaaattgggcggagtggaagatccggatgtgtacccgacacggggacatgtggttctagttaaagctccgcatgttaccgaaacccgtatcttgaacggcaaaaattcggataccgggatcattcccaggccgctgaatgggggcgttatctgtgggggctttatgcagcccggtaactgggatcgtgaaattcacccagaagacacactggatattttaaaacgtacctctgctttgatgccagaactgttccatgggaaaggcccggaaggcgcagaaatcattcaggaatgcgtgggattccgccccagccgtaaaggtggcgccagagttgaactggatgtagtacctggtacatccgtcccgcttgtacacgactatggcgcttcgggaacgggttaccaagccggttatggtatggcacttgatagcgtgatgctcgcattaccaaaaatcaaacttgcctaa

SEQ ID NO: 40 - DNA, 1047 bp, Artificial Sequence
  Description of Artificial Sequence Synthetic polynucleotide
atgactaaggagaacaagcctcgcgacattgtcatcgtgggggcgggtgttattggtctgacgactgcctggattctgagcgatctgggcctggcgccacgaataaaagtcattgcgaaatatacgcctgaggatcgctctgttgagtacacgtcaccttgggctggggcgaatttttgcagtataagtgcaactgatgacaacgctttacggtgggacaagattacgtaccaccgttttgcgtatctcgcgaaaaccagaccggaagccggtattcgctttgcggatctccgcgagttatgggagtacgagccgaaacatgataaaattcgttcctggaatacttacgtgcgcgacttcaaagtgatcccggaaaaggacctgccgggtgagtgcatctatggccataaggcaaccaccttccttatcaacgccccgcattatctgaattatatgtataaactgttgatagaagcaggtgtagaatttgaaaaaaaggaactgtctcacattaaagagaccgtcgaagagacaccagaagcgagtgtcgtgtttaattgtacgggcctgtgggcctcaaaattgggcggagtggaagatccggatgtgtacccgacacggggacatgtggttctagttaaagctccgcatgttaccgaaacccgtatcttgaacggcaaaaattcggatacccatatcattcccaggccgctgaatgggggcgttatctgtgggggctttatgcagcccggtaactgggatcgtgaaattcacccagaagacacactggatattttaaaacgtacctctgctttgatgccagaactgttccatgggaaaggcccggaaggcgcagaaatcattcaggaatgcgtgggattccgccccagccgtaaaggtggcgccagagttgaactggatgtagtacctggtacatccgtcccgcttgtacacgactatggcgcttcgggaacgggttaccaagccggttatggtatggcacttgatagcgtgatgctcgcattaccaaaaatcaaacttgcctaa

SEQ ID NO: 41 - DNA, 1047 bp, Artificial Sequence
  Description of Artificial Sequence Synthetic polynucleotide
atgactaaggagaacaagcctcgcgacattgtcatcgtgggggcgggtgttattggtctgacgactgcctggattctgagcgatctgggcctggcgccacgaataaaagtcattgcgaaatatacgcctgaggatcgctctgttgagtacacgtcaccttgggctggggcgaatttttgcagtataagtgcaactgatgacaacgctttacggtgggacaagattacgtaccaccgttttgcgtatctcgcgaaaaccagaccggaagccggtattcgctttgcggatctccgcgagttatgggagtacgagccgaaacatgataaaattcgttcctggaatacttacgtgcgcgacttcaaagtgatcccggaaaaggacctgccgggtgagtgcatctatggccataaggcaaccaccttccttatcaacgccccgcattatctgaattatatgtataaactgttgatagaagcaggtgtagaatttgaaaaaaaggaactgtctcacattaaagagaccgtcgaagagacaccagaagcgagtgtcgtgtttaattgtacgggcctgtgggcctcaaaattgggcggagtggaagatccggatgtgtacccgacacggggacatgtggttctagttaaagctccgcatgttaccgaaacccgtatcttgaacggcaaaaattcggataccattatcattcccaggccgctgaatgggggcgttatctgtgggggctttatgcagcccggtaactgggatcgtgaaattcacccagaagacacactggatattttaaaacgtacctctgctttgatgccagaactgttccatgggaaaggcccggaaggcgcagaaatcattcaggaatgcgtgggattccgccccagccgtaaaggtggcgccagagttgaactggatgtagtacctggtacatccgtcccgcttgtacacgactatggcgcttcgggaacgggttaccaagccggttatggtatggcacttgatagcgtgatgctcgcattaccaaaaatcaaacttgcctaa

SEQ ID NO: 42 - DNA, 1047 bp, Artificial Sequence
  Description of Artificial Sequence Synthetic polynucleotide
atgactaaggagaacaagcctcgcgacattgtcatcgtgggggcgggtgttattggtctgacgactgcctggattctgagcgatctgggcctggcgccacgaataaaagtcattgcgaaatatacgcctgaggatcgctctgttgagtacacgtcaccttgggctggggcgaatttttgcagtataagtgcaactgatgacaacgctttacggtgggacaagattacgtaccaccgttttgcgtatctcgcgaaaaccagaccggaagccggtattcgctttgcggatctccgcgagttatgggagtacgagccgaaacatgataaaattcgttcctggaatacttacgtgcgcgacttcaaagtgatcccggaaaaggacctgccgggtgagtgcatctatggccataaggcaaccaccttccttatcaacgccccgcattatctgaattatatgtataaactgttgatagaagcaggtgtagaatttgaaaaaaaggaactgtctcacattaaagagaccgtcgaagagacaccagaagcgagtgtcgtgtttaattgtacgggcctgtgggcctcaaaattgggcggagtggaagatccggatgtgtacccgacacggggacatgtggttctagttaaagctccgcatgttaccgaaacccgtatcttgaacggcaaaaattcggatacctctatcattcccaggccgctgaatgggggcgttatctgtgggggctttatgcagcccggtaactgggatcgtgaaattcacccagaagacacactggatattttaaaacgtacctctgctttgatgccagaactgttccatgggaaaggcccggaaggcgcagaaatcattcaggaatgcgtgggattccgccccagccgtaaaggtggcgccagagttgaactggatgtagtacctggtacatccgtcccgcttgtacacgactatggcgcttcgggaacgggttaccaagccggttatggtatggcacttgatagcgtgatgctcgcattaccaaaaatcaaacttgcctaa

SEQ ID NO: 43 - DNA, 1047 bp, Artificial Sequence
  Description of Artificial Sequence Synthetic polynucleotide
atgactaaggagaacaagcctcgcgacattgtcatcgtgggggcgggtgttattggtctgacgactgcctggattctgagcgatctgggcctggcgccacgaataaaagtcattgcgaaatatacgcctgaggatcgctctgttgagtacacgtcaccttgggctggggcgaatttttgcagtataagtgcaactgatgacaacgctttacggtgggacaagattacgtaccaccgttttgcgtatctcgcgaaaaccagaccggaagccggtattcgctttgcggatctccgcgagttatgggagtacgagccgaaacatgataaaattcgttcctggaatacttacgtgcgcgacttcaaagtgatcccggaaaaggacctgccgggtgagtgcatctatggccataaggcaaccaccttccttatcaacgccccgcattatctgaattatatgtataaactgttgatagaagcaggtgtagaatttgaaaaaaaggaactgtctcacattaaagagaccgtcgaagagacaccagaagcgagtgtcgtgtttaattgtacgggcctgtgggcctcaaaattgggcggagtggaagatccggatgtgtacccgacacggggacatgtggttctagttaaagctccgcatgttaccgaaacccgtatcttgaacggcaaaaattcggataccatgatcattcccaggccgctgaatgggggcgttatctgtgggggctttatgcagcccggtaactgggatcgtgaaattcacccagaagacacactggatattttaaaacgtacctctgctttgatgccagaactgttccatgggaaaggcccggaaggcgcagaaatcattcaggaatgcgtgggattccgccccagccgtaaaggtggcgccagagttgaactggatgtagtacctggtacatccgtcccgcttgtacacgactatggcgcttcgggaacgggttaccaagccggttatggtatggcacttgatagcgtgatgctcgcattaccaaaaatcaaacttgcctaa

SEQ ID NO: 44 - DNA, 1047 bp, Artificial Sequence
  Description of Artificial Sequence Synthetic polynucleotide
atgactaaggagaacaagcctcgcgacattgtcatcgtgggggcgggtgttattggtctgacgactgcctggattctgagcgatctgggcctggcgccacgaataaaagtcattgcgaaatatacgcctgaggatcgctctgttgagtacacgtcaccttgggctggggcgaatttttgcagtataagtgcaactgatgacaacgctttacggtgggacaagattacgtaccaccgttttgcgtatctcgcgaaaaccagaccggaagccggtattcgctttgcggatctccgcgagttatgggagtacgagccgaaacatgataaaattcgttcctggaatacttacgtgcgcgacttcaaagtgatcccggaaaaggacctgccgggtgagtgcatctatggccataaggcaaccaccttccttatcaacgccccgcattatctgaattatatgtataaactgttgatagaagcaggtgtagaatttgaaaaaaaggaactgtctcacattaaagagaccgtcgaagagacaccagaagcgagtgtcgtgtttaattgtacgggcctgtgggcctcaaaattgggcggagtggaagatccggatgtgtacccgacacggggacatgtggttctagttaaagctccgcatgttaccgaaacccgtatcttgaacggcaaaaattcggatacccagatcattcccaggccgctgaatgggggcgttatctgtgggggctttatgcagcccggtaactgggatcgtgaaattcacccagaagacacactggatattttaaaacgtacctctgctttgatgccagaactgttccatgggaaaggcccggaaggcgcagaaatcattcaggaatgcgtgggattccgccccagccgtaaaggtggcgccagagttgaactggatgtagtacctggtacatccgtcccgcttgtacacgactatggcgcttcgggaacgggttaccaagccggttatggtatggcacttgatagcgtgatgctcgcattaccaaaaatcaaacttgcctaa

SEQ ID NO: 45 - DNA, 1047 bp, Artificial Sequence
  Description of Artificial Sequence Synthetic polynucleotide
atgactaaggagaacaagcctcgcgacattgtcatcgtgggggcgggtgttattggtctgacgactgcctggattctgagcgatctgggcctggcgccacgaataaaagtcattgcgaaatatacgcctgaggatcgctctgttgagtacacgtcaccttgggctggggcgaatttttgcagtataagtgcaactgatgacaacgctttacggtgggacaagattacgtaccaccgttttgcgtatctcgcgaaaaccagaccggaagccggtattcgctttgcggatctccgcgagttatgggagtacgagccgaaacatgataaaattcgttcctggaatacttacgtgcgcgacttcaaagtgatcccggaaaaggacctgccgggtgagtgcatctatggccataaggcaaccaccttccttatcaacgccccgcattatctgaattatatgtataaactgttgatagaagcaggtgtagaatttgaaaaaaaggaactgtctcacattaaagagaccgtcgaagagacaccagaagcgagtgtcgtgtttaattgtacgggcctgtgggcctcaaaattgggcggagtggaagatccggatgtgtacccgacacggggacatgtggttctagttaaagctccgcatgttaccgaaacccgtatcttgaacggcaaaaattcggatacctgtatcattcccaggccgctgaatgggggcgttatctgtgggggctttatgcagcccggtaactgggatcgtgaaattcacccagaagacacactggatattttaaaacgtacctctgctttgatgccagaactgttccatgggaaaggcccggaaggcgcagaaatcattcaggaatgcgtgggattccgccccagccgtaaaggtggcgccagagttgaactggatgtagtacctggtacatccgtcccgcttgtacacgactatggcgcttcgggaacgggttaccaagccggttatggtatggcacttgatagcgtgatgctcgcattaccaaaaatcaaacttgcctaa

SEQ ID NO: 46 - DNA, 1047 bp, Artificial Sequence
  Description of Artificial Sequence Synthetic polynucleotide
atgactaaggagaacaagcctcgcgacattgtcatcgtgggggcgggtgttattggtctgacgactgcctggattctgagcgatctgggcctggcgccacgaataaaagtcattgcgaaatatacgcctgaggatcgctctgttgagtacacgtcaccttgggctggggcgaatttttgcagtataagtgcaactgatgacaacgctttacggtgggacaagattacgtaccaccgttttgcgtatctcgcgaaaaccagaccggaagccggtattcgctttgcggatctccgcgagttatgggagtacgagccgaaacatgataaaattcgttcctggaatacttacgtgcgcgacttcaaagtgatcccggaaaaggacctgccgggtgagtgcatctatggccataaggcaaccaccttccttatcaacgccccgcattatctgaattatatgtataaactgttgatagaagcaggtgtagaatttgaaaaaaaggaactgtctcacattaaagagaccgtcgaagagacaccagaagcgagtgtcgtgtttaattgtacgggcctgtgggcctcaaaattgggcggagtggaagatccggatgtgtacccgacacggggacatgtggttctagttaaagctccgcatgttaccgaaacccgtatcttgaacggcaaaaattcggataccgctatcattcccaggccgctgaatgggggcgttatctgtgggggctttatgcagcccggtaactgggatcgtgaaattcacccagaagacacactggatattttaaaacgtacctctgctttgatgccagaactgttccatgggaaaggcccggaaggcgcagaaatcattcaggaatgcgtgggattccgccccagccgtaaaggtggcgccagagttgaactggatgtagtacctggtacatccgtcccgcttgtacacgactatggcgcttcgggaacgggttaccaagccggttatggtatggcacttgatagcgtgatgctcgcattaccaaaaatcaaacttgcctaa

SEQ ID NO: 47 - DNA, 1071 bp, Artificial Sequence
  Description of Artificial Sequence Synthetic polynucleotide
atggctaaaatcgttgttattggtgccggtgttgccggtttaactacagctcttcaacttcttcgtaaaggacatgaggttacaattgtgtccgagtttacgcccggtgatcttagtatcggatatacctcgccttgggcaggtgccaactggctcacattttacgatggaggcaagttagccgactacgatgccgtctcttatcctatcttgcgagagctggctcgaagcagccccgaggctggaattcgactcatcaaccaacgctcccatgttctcaagcgtgatcttcctaaactggaaggtgccatgtcggccatctgtcaacgcaacccctggttcaaaaacacagtcgattctttcgagattatcgaggacaggtccaggattgtccacgatgatgtggcttatctagtcgaatttgcttccgtttgtatccacaccggagtctacttgaactggctgatgtcccaatgcttatcgctcggcgccacggtggttaaacgtcgagtgaaccatatcaaggatgccaatttactacactcctcaggatcacgccccgacgtgattgtcaactgtagtggtctctttgcccggttcttgggaggcgtcgaggacaagaagatgtaccctattcgaggacaagtcgtccttgttcgaaactctcttccttttatggcctccttttccagcactcctgaaaaagaaaatgaagacgaagctctatatatcatgacccgattcgatggtacttctatcattggcggttgtttccaacccaacaactggtcatccgaacccgatccttctctcacccatcgaatcctgtctagagccctcgaccgattcccggaactgaccaaagatggccctcttgacattgtgcgcgaatgcgttggccaccgtcctggtagagagggcggtccccgagtagaattagagaagatccccggcgttggctttgttgtccataactatggtgccgccggtgctggttaccagtcctcttacggcatggctgatgaagctgtttcttacgtcgaaagagctcttactcgtccaaacctttag

SEQ ID NO: 48 - DNA, 1071 bp, Artificial Sequence
  Description of Artificial Sequence Synthetic polynucleotide
atggctaaaatcgttgttattggtgccggtgttgccggtttaactacagctcttcaacttcttcgtaaaggacatgaggttacaattgtgtccgagtttacgcccggtgatcttagtatcggatatacctcgccttgggcaggtgccaactggctcacattttacgatggaggcaagttagccgactacgatgccgtctcttatcctatcttgcgagagctggctcgaagcagccccgaggctggaattcgactcatcaaccaacgctcccatgttctcaagcgtgatcttcctaaactggaaggtgccatgtcggccatctgtcaacgcaacccctggttcaaaaacacagtcgattctttcgagattatcgaggacaggtccaggattgtccacgatgatgtggcttatctagtcgaatttgcttccgtttgtatccacaccggagtctacttgaactggctgatgtcccaatgcttatcgctcggcgccacggtggttaaacgtcgagtgaaccatatcaaggatgccaatttactacactcctcaggatcacgccccgacgtgattgtcaactgtagtggtctctttgcccggttcttgggaggcgtcgaggacaagaagatgtaccctattcgaggacaagtcgtccttgttcgaaactctcttccttttatggcctccttttccagcactcctgaaaaagaaaatgaagacgaagctctacatatcatgacccgattcgatggtacttctatcattggcggttgtttccaacccaacaactggtcatccgaacccgatccttctctcacccatcgaatcctgtctagagccctcgaccgattcccggaactgaccaaagatggccctcttgacattgtgcgcgaatgcgttggccaccgtcctggtagagagggcggtccccgagtagaattagagaagatccccggcgttggctttgttgtccataactatggtgccgccggtgctggttaccagtcctcttacggcatggctgatgaagctgtttcttacgtcgaaagagctcttactcgtccaaacctttag

SEQ ID NO: 49 - DNA, 1071 bp, Artificial Sequence
  Description of Artificial Sequence Synthetic polynucleotide
atggctaaaatcgttgttattggtgccggtgttgccggtttaactacagctcttcaacttcttcgtaaaggacatgaggttacaattgtgtccgagtttacgcccggtgatcttagtatcggatatacctcgccttgggcaggtgccaactggctcacattttacgatggaggcaagttagccgactacgatgccgtctcttatcctatcttgcgagagctggctcgaagcagccccgaggctggaattcgactcatcaaccaacgctcccatgttctcaagcgtgatcttcctaaactggaaggtgccatgtcggccatctgtcaacgcaacccctggttcaaaaacacagtcgattctttcgagattatcgaggacaggtccaggattgtccacgatgatgtggcttatctagtcgaatttgcttccgtttgtatccacaccggagtctacttgaactggctgatgtcccaatgcttatcgctcggcgccacggtggttaaacgtcgagtgaaccatatcaaggatgccaatttactacactcctcaggatcacgccccgacgtgattgtcaactgtagtggtctctttgcccggttcttgggaggcgtcgaggacaagaagatgtaccctattcgaggacaagtcgtccttgttcgaaactctcttccttttatggcctccttttccagcactcctgaaaaagaaaatgaagacgaacacctagtcatcatgacccgattcgatggtacttctatcattggcggttgtttccaacccaacaactggtcatccgaacccgatccttctctcacccatcgaatcctgtctagagccctcgaccgattcccggaactgaccaaagatggccctcttgacattgtgcgcgaatgcgttggccaccgtcctggtagagagggcggtccccgagtagaattagagaagatccccggcgttggctttgttgtccataactatggtgccgccggtgctggttaccagtcctcttacggcatggctgatgaagctgtttcttacgtcgaaagagctcttactcgtccaaacctttag

SEQ ID NO: 50 - DNA, 1071 bp, Artificial Sequence
  Description of Artificial Sequence Synthetic polynucleotide
atggctaaaatcgttgttattggtgccggtgttgccggtttaactacagctcttcaacttcttcgtaaaggacatgaggttacaattgtgtccgagtttacgcccggtgatcttagtatcggatatacctcgccttgggcaggtgccaactggctcacattttacgatggaggcaagttagccgactacgatgccgtctcttatcctatcttgcgagagctggctcgaagcagccccgaggctggaattcgactcatcaaccaacgctcccatgttctcaagcgtgatcttcctaaactggaaggtgccatgtcggccatctgtcaacgcaacccctggttcaaaaacacagtcgattctttcgagattatcgaggacaggtccaggattgtccacgatgatgtggcttatctagtcgaatttgcttccgtttgtatccacaccggagtctacttgaactggctgatgtcccaatgcttatcgctcggcgccacggtggttaaacgtcgagtgaaccatatcaaggatgccaatttactacactcctcaggatcacgccccgacgtgattgtcaactgtagtggtctctttgcccggttcttgggaggcgtcgaggacaagaagatgtaccctattcgaggacaagtcgtccttgttcgaaactctcttccttttatggcctccttttccagcactcctgaaaaagaaaatgaagacgaaatgctagtgatcatgacccgattcgatggtacttctatcattggcggttgtttccaacccaacaactggtcatccgaacccgatccttctctcacccatcgaatcctgtctagagccctcgaccgattcccggaactgaccaaagatggccctcttgacattgtgcgcgaatgcgttggccaccgtcctggtagagagggcggtccccgagtagaattagagaagatccccggcgttggctttgttgtccataactatggtgccgccggtgctggttaccagtcctcttacggcatggctgatgaagctgtttcttacgtcgaaagagctcttactcgtccaaacctttag

SEQ ID NO: 51 - DNA, 1071 bp, Artificial Sequence
  Description of Artificial Sequence Synthetic polynucleotide
atggctaaaatcgttgttattggtgccggtgttgccggtttaactacagctcttcaacttcttcgtaaaggacatgaggttacaattgtgtccgagtttacgcccggtgatcttagtatcggatatacctcgccttgggcaggtgccaactggctcacattttacgatggaggcaagttagccgactacgatgccgtctcttatcctatcttgcgagagctggctcgaagcagccccgaggctggaattcgactcatcaaccaacgctcccatgttctcaagcgtgatcttcctaaactggaaggtgccatgtcggccatctgtcaacgcaacccctggttcaaaaacacagtcgattctttcgagattatcgaggacaggtccaggattgtccacgatgatgtggcttatctagtcgaatttgcttccgtttgtatccaccccggagtctacttgaactggctgctgtcccaatgcttatcgctcggcgccacggtggttaaacgtcgagtgaaccatatcaaggatgccaatttactacactcctcaggatcacgccccgacgtgattgtcaactgtagtggtctctttgcccggttcttgggaggcgtcgaggacaagaagctgtaccctattcgaggacaagtcgtccttgttcgaaactctcttccttttatggcctccttttccagcactcctgaaaaagaaaatgaagacgaaatgctagtgatcatgacccgattcgatggtacttctatcattggcggttgtttccaacccaacaactggtcatccgaacccgatccttctctcacccatcgaatcctgtctagagccctcgaccgattcccggaactgaccaaagatggccctcttgacattgtgcgcgaatgcgttggccaccgtcctggtagagagggcggtccccgagtagaattagagaagatccccggcgttggctttgttgtccataactatggtgccgccggtgctggttaccagtcctcttacggcatggctgatgaagctgtttcttacgtcgaaagagctcttactcgtccaaacctttag

SEQ ID NO: 52 - DNA, 1071 bp, Artificial Sequence
  Description of Artificial Sequence Synthetic polynucleotide
atggctaaaatcgttgttattggtgccggtgttgccggtttaactacagctcttcaacttcttcgtaaaggacatgaggttacaattgtgtccgagtttacgcccggtgatcttagtatcagatatacctcgccttgggcaggtgccaactggctcacattttacgatggaggcaagttagccgactacgatgccgtctcttatcctatcttgcgagagctggctcgaagcagccccgaggctggaattcgactcatcaaccaacgctcccatgttctcaagcgtgatcttcctaaactggaaggtgccatgtcgaccatctgtcaacgcaacccctggttcaaaaacacagtcgattctttcgagattatcgaggacaggtccaggattgtccacgatgatgtggcttatctagtcgaatttgcttccgtttgtatccaccccggagtctacttgaactggctgctgtcccaatgcttatcgctcggcgccacggtggttaaacgtcgagtgaaccatatcaaggatgccaatttactacactcctcaggatcacgccccgacgtgattgtcaactgtagtggtctctttgcccggttcttgggaggcgtcgaggacaagaagctgtaccctattcgaggacaagtcgtccttgttcgaaactctcttccttttatggcctccttttccagcactcctgaaaaagaaaatgaagacgaaatgctagtgatcatgacccgattcgatggtacttctatcattggcggttgtttccaacccaacaactggtcatccgaacccgatccttctctcacccatcgaatcctgtctagagccctcgaccgattcccggaactgaccaaagatggccctcttgacattgtgcgcgaatgcgttggccaccgtcctggtagagagggcggtccccgagtagaattagagaagatccccggcgttggctttgttgtccataactatggtgccgccggtgctggttaccagtcctcttacggcatggctgatgaagctgtttcttacgtcgaaagagctcttactcgtccaaacctttag

SEQ ID NO: 53 - DNA, 1071 bp, Artificial Sequence
  Description of Artificial Sequence Synthetic polynucleotide
atggctaaaatcgttgttattggtgccggtgttgccggtttaactacagctcttcaacttcttcgtaaaggacatgaggttacaattgtgtccgagtttacgcccggtgatcttagtatcagatatacctcgccttgggcaggtgccaactggctcacatttagcgatggaggcaagttagccgactacgatgccgtctcttatcctatcttgcgagagctggctcgaagcagccccgaggctggaattcgactcatcaaccaacgctcccatgttctcaagcgtgatcttcctaaactggaaggtgccatgtcgaccatctgtcaacgcaacccctggttcaaaaacacagtcgattctttcgagattatcgaggacaggtccaggattgtccacgatgatgtggcttatctagtcgaatttgcttccgtttgtatccaccccggagtctacttgaactggctgctgtcccaatgcttatcgctcggcgccacggtggttaaacgtcgagtgaaccatatcaaggatgccaatttactacactcctcaggatcacgccccgacgtgattgtcaactgtagtggtctctttgcccggttcttgggaggcgtcgaggacaagaagctgtaccctattcgaggacaagtcgtccttgttcgaaactctcttccttttatggcctccttttccagcactcctgaaaaagaaaatgaagacgaaatgctagtgatcatgacccgattcgatggtacttctatcattggcggttgtttccaacccaacaactggtcatccgaacccgatccttctctcacccatcgaatcctgtctagagccctcgaccgattcccggaactgaccaaagatggccctcttgacattgtgcgcgaatgcgttggccaccgtcctggtagagagggcggtccccgagtagaattagagaagatccccggcgttggctttgttgtccataactatggtgccgccggtgctggttaccagtcctcttacggcatggctgatgaagctgtttcttacgtcgaaagagctcttactcgtccaaacctttag

SEQ ID NO: 54 - DNA, 1071 bp, Artificial Sequence
  Description of Artificial Sequence Synthetic polynucleotide
atggctaaaatcgttgttattggtgccggtgttgccggtttaactacagctcttcaacttcttcgtaaaggacatgaggttacaattgtgtccgagtttacgcccggtgatcttagtatcagatatacctcgccttgggcaggtgccaactggctcacatttagcgaaggaggcaagttagccgactacgatgccgtctcttatcctatcttgcgagagctggctcgaagcagccccgaggctggaattcgactcatcaaccaacgctcccatgttctcaagcgtgatcttcctaaactggaaggtgccatgtcgaccatctgtcaacgcaacccctggttcaaaaacacagtcgattctttcgagattatcgaggacaggtccaggattgtccacgatgatgtggcttatctagtcgaatttgcttccgtttgtatccaccccggagtctacttgaactggctgctgtcccaatgcttatcgctcggcgccacggtggttaaacgtcgagtgaaccatatcaaggatgccaatttactacactcctcaggatcacgccccgacgtgattgtcaactgtagtggtctctttgcccggttcttgggaggcgtcgaggacaagaagctgtaccctattcgaggacaagtcgtccttgttcgaaactctcttccttttatggcctccttttccagcactcctgaaaaagaaaatgaagacgaaatgctagtgatcatgacccgattcgatggtacttctatcattggcggttgtttccaacccaacaactggtcatccgaacccgatccttctctcacccatcgaatcctgtctagagccctcgaccgattcccggaactgaccaaagatggccctcttgacattgtgcgcgaatgcgttggccaccgtcctggtagagagggcggtccccgagtagaattagagaagatccccggcgttggctttgttgtccataactatggtgccgccggtgctggttaccagtcctcttacggcatggctgatgaagctgtttcttacgtcgaaagagctcttactcgtccaaacctttag

SEQ ID NO: 55 - DNA, 1071 bp, Artificial Sequence
  Description of Artificial Sequence Synthetic polynucleotide
atggctaaaatcgttgttattggtgccggtgttgccggtttaactacagctcttcaacttcttcgtaaaggacatgaggttacaattgtgtccgagtttacgcccggtgatcttagtatcagatatacctcgccttgggcaggtgccaactggctcacatttagcgaaggaggcaagttagccgactacgatgccgtctcttatcctatcttgcgagagctggctcgaagcagccccgaggctggaattcgactcatcaaccaacgctcccatgttctcaagcgtgatcttcctaaactggaaggtcccatgtcgaccatctgtcaacgcaacccctggttcaaaaacacagtcgattctttcgagattatcgaggacaggtccaggattgtccacgatgatgtggcttatctagtcgaatttgcttccgtttgtatccaccccggagtctacttgaactggctgctgtcccaatgcttatcgctcggcgccacggtggttaaacgtcgagtgaaccatatcaaggatgccaatttactacactcctcaggatcacgccccgacgtgattgtcaactgtagtggtctctttgcccggttcttgggaggcgtcgaggacaagaagctgtaccctattcgaggacaagtcgtccttgttcgaaactctcttccttttatggcctccttttccagcactcctgaaaaagaaaatgaagacgaaatgctagtgatcatgacccgattcgatggtacttctatcattggcggttgtttccaacccaacaactggtcatccgaacccgatccttctctcacccatcgaatcctgtctagagccctcgaccgattcccggaactgaccaaagatggccctcttgacattgtgcgcgaatgcgttggccaccgtcctggtagagagggcggtccccgagtagaattagagaagatccccggcgttggctttgttgtccataactatggtgccgccggtgctggttaccagtcctcttacggcatggctgatgaagctgtttcttacgtcgaaagagctcttactcgtccaaacctttag

SEQ ID NO: 56 - DNA, 1071 bp, Artificial Sequence
  Description of Artificial Sequence Synthetic polynucleotide
atggctaaaatcgttgttattggtgccggtgttgccggtttaactacagctcttcaacttcttcgtaaaggacatgaggttacaattgtgtccgagtttacgcccggtgatcttagtatcagatatacctcgccttgggcaggtgccaactggctcacattttacgatggaggcaagttagccgactacgatgccgtctcttatcctatcttgcgagagctggctcgaagcagccccgaggctggaattcgactcatcaaccaacgctcccatgttctcaagcgtgatcttcctaaactggaaggtgccatgtcgaccatctgtcaacgcaacccctggttcaaaaacacagtcgattctttcgagattatcgaggacaggtccaggattgtccacgatgatgtggcttatctagtcgaatttgcttccgtttgtatccaccccggagtctacttgaactggctgctgtcccaatgcttatcgctcggcgccacggtggttaaacgtcgagtgaaccatatcaaggatgccaatttactacactcctcaggatcacgccccgacgtgattgtcaactgtagtggtctctttgcccggttcttgggaggcgtcgaggacaagaagctgtaccctattcgaggacaagtcgtccttgttcgaaactctcttccttttatggcctccttttccagcactcctgaaaaagaaaatgaagacgaagctctatatatcatgacccgattcgatggtacttctatcattggcggttgtttccaacccaacaactggtcatccgaacccgatccttctctcacccatcgaatcctgtctagagccctcgaccgattcccggaactgaccaaagatggccctcttgacattgtgcgcgaatgcgttggccaccgtcctggtagagagggcggtccccgagtagaattagagaagatccccggcgttggctttgttgtccataactatggtgccgccggtgctggttaccagtcctcttacggcatggctgatgaagctgtttcttacgtcgaaagagctcttactcgtccaaacctttag

SEQ ID NO: 57 - DNA, 1071 bp, Artificial Sequence
  Description of Artificial Sequence Synthetic polynucleotide
atggctaaaatcgttgttattggtgccggtgttgccggtttaactacagctcttcaacttcttcgtaaaggacatgaggttacaattgtgtccgagtttacgcccggtgatcttagtatcagatatacctcgccttgggcaggtgccaactggctcacattttacgatggaggcaagttagccgactacgatgccgtctcttatcctatcttgcgagagctggctcgaagcagccccgaggctggaattcgactcatcaaccaacgctcccatgttctcaagcgtgatcttcctaaactggaaggtgccatgtcgaccatctgtcaacgcaacccctggttcaaaaacacagtcgattctttcgagattatcgaggacaggtccaggattgtccacgatgatgtggcttatctagtcgaatttgcttccgtttgtatccaccccggagtctacttgaactggctgctgtcccaatgcttatcgctcggcgccacggtggttaaacgtcgagtgaaccatatcaaggatgccaatttactacactcctcaggatcacgccccgacgtgattgtcaactgtagtggtctctttgcccggttcttgggaggcgtcgaggacaagaagctgtaccctattcgaggacaagtcgtccttgttcgaaactctcttccttttatggcctggttttccagcactcctgaaaaagaaaatgaagacgaagctctatatatcatgacccgattcgatggtacttctatcattggcggttgtttccaacccaacaactggtcatccgaacccgatccttctctcacccatcgaatcctgtctagagccctcgaccgattcccggaactgaccaaagatggccctcttgacattgtgcgcgaatgcgttggccaccgtcctggtagagagggcggtccccgagtagaattagagaagatccccggcgttggctttgttgtccataactatggtgccgccggtgctggttaccagtcctcttacggcatggctgatgaagctgtttcttacgtcgaaagagctcttactcgtccaaacctttag

SEQ ID NO: 58 - DNA, 1071 bp, Artificial Sequence
  Description of Artificial Sequence Synthetic polynucleotide
atggctaaaatcgttgttattggtgccggtgttgccggtttaactacagctcttcaacttcttcgtaaaggacatgaggttacaattgtgtccgagtttacgcccggtgatcttagtatcagatatacctcgccttgggcaggtgccaactggctcacattttacgatggaggcaagttagccgactacgatgccgtctcttatcctatcttgcgagagctggctcgaagcagccccgaggctggaattcgactcatcaaccaacgctcccatgttctcaagcgtgatcttcctaaactggaaggtgccatgtcgaccatctgtcaacgcaacccctggttcaaaaacacagtcgattctttcgagattatcgaggacaggtccaggattgtccacgatgatgtggcttatctagtcgaatttgcttccgtttgtatccaccccggagtctacttgaactggctgctgtcccaatgcttatcgctcggcgccacggtggttaaacgtcgagtgaaccatatcaaggatgccaatttactacactcctcaggatcacgccccgacgtgattgtcaactgtagtggtctctttgcccggttcttgggaggcgtcgaggacaagaagctgtaccctattcgaggacaagtcgtccttgttcgaaactctcttccttttatggcctacttttccagcactcctgaaaaagaaaatgaagacgaagctctatatatcatgacccgattcgatggtacttctatcattggcggttgtttccaacccaacaactggtcatccgaacccgatccttctctcacccatcgaatcctgtctagagccctcgaccgattcccggaactgaccaaagatggccctcttgacattgtgcgcgaatgcgttggccaccgtcctggtagagagggcggtccccgagtagaattagagaagatccccggcgttggctttgttgtccataactatggtgccgccggtgctggttaccagtcctcttacggcatggctgatgaagctgtttcttacgtcgaaagagctcttactcgtccaaacctttag

SEQ ID NO: 59 - DNA, 1071 bp, Artificial Sequence
  Description of Artificial Sequence Synthetic polynucleotide
atggctaaaatcgttgttattggtgccggtgttgccggtttaactacagctcttcaacttcttcgtaaaggacatgaggttacaattgtgtccgagtttacgcccggtgatcttagtatcagatatacctcgccttgggcaggtgccaactggctcacattttacgatggaggcaagttagccgactacgatgccgtctcttatcctatcttgcgagagctggctcgaagcagccccgaggctggaattcgactcatcaaccaacgctcccatgttctcaagcgtgatcttcctaaactggaaggtgccatgtcgaccatctgtcaacgcaacccctggttcaaaaacacagtcgattctttcgagattatcgaggacaggtccaggattgtccacgatgatgtggcttatctagtcgaatttgcttccgtttgtatccaccccggagtctacttgaactggctgctgtcccaatgcttatcgctcggcgccacggtggttaaacgtcgagtgaaccatatcaaggatgccaatttactacactcctcaggatcacgccccgacgtgattgtcaactgtagtggtctctttgcccggttcttgggaggcgtcgaggacaagaagctgtaccctattcgaggacaagtcgtccttgttcgaaactctcttccttttatggccctgttttccagcactcctgaaaaagaaaatgaagacgaagctctatatatcatgacccgattcgatggtacttctatcattggcggttgtttccaacccaacaactggtcatccgaacccgatccttctctcacccatcgaatcctgtctagagccctcgaccgattcccggaactgaccaaagatggccctcttgacattgtgcgcgaatgcgttggccaccgtcctggtagagagggcggtccccgagtagaattagagaagatccccggcgttggctttgttgtccataactatggtgccgccggtgctggttaccagtcctcttacggcatggctgatgaagctgtttcttacgtcgaaagagctcttactcgtccaaacctttag

SEQ ID NO: 60 - DNA, 1071 bp, Artificial Sequence
  Description of Artificial Sequence Synthetic polynucleotide
atggctaaaatcgttgttattggtgccggtgttgccggtttaactacagctcttcaacttcttcgtaaaggacatgaggttacaattgtgtccgagtttacgcccggtgatcttagtatcagatatacctcgccttgggcaggtgccaactggctcacatttagcgaaggaggcaagttagccgactacgatgccgtctcttatcctatcttgcgagagctggctcgaagcagccccgaggctggaattcgactcatcaaccaacgctcccatgttctcaagcgtgatcttcctaaactggaaggtcccatgtcgaccatctgtcaacgcaacccctggttcaaaaacacagtcgattctttcgagattatcgaggacaggtccaggattgtccacgatgatgtggcttatctagtcgaatttgcttccgtttgtatccaccccggagtctacttgaactggctgctgtcccaatgcttatcgctcggcgccacggtggttaaacgtcgagtgaaccatatcaaggatgccaatttactacactcctcaggatcacgccccgacgtgattgtcaactgtagtggtctctttgcccggttcttgggaggcgtcgaggacaagaagctgtaccctattcgaggacaagtcgtccttgttcgaaactctcttccttttatggccatcttttccagcactcctgaaaaagaaaatgaagacgaaatgctagtgatcatgacccgattcgatggtacttctatcattggcggttgtttccaacccaacaactggtcatccgaacccgatccttctctcacccatcgaatcctgtctagagccctcgaccgattcccggaactgaccaaagatggccctcttgacattgtgcgcgaatgcgttggccaccgtcctggtagagagggcggtccccgagtagaattagagaagatccccggcgttggctttgttgtccataactatggtgccgccggtgctggttaccagtcctcttacggcatggctgatgaagctgtttcttacgtcgaaagagctcttactcgtccaaacctttag

SEQ ID NO: 61 - DNA, 1071 bp, Artificial Sequence
  Description of Artificial Sequence Synthetic polynucleotide
atggctaaaatcgttgttattggtgccggtgttgccggtttaactacagctcttcaacttcttcgtaaaggacatgaggttacaattgtgtccgagtttacgcccggtgatcttagtatcagatatacctcgccttgggcaggtgccaactggctcacatttagcgaaggaggcaagttagccgactacgatgccgtctcttatcctatcttgcgagagctggctcgaagcagccccgaggctggaattcgactcatcaaccaacgctcccatgttctcaagcgtgatcttcctaaactggaaggtcccatgtcgaccatctgtcaacgcaacccctggttcaaaaacacagtcgattctttcgagattatcgaggacaggtccaggattgtccacgatgatgtggcttatctagtcgaatttgcttccgtttgtatccaccccggagtctacttgaactggctgctgtcccaatgcttatcgctcggcgccacggtggttaaacgtcgagtgaaccatatcaaggatgccaatttactacactcctcaggatcacgccccgacgtgattgtcaactgtagtggtctctttgcccggttcttgggaggcgtcgaggacaagaagctgtaccctattcgaggacaagtcgtccttgttcgaaactctcttccttttatggccatgttttccagcactcctgaaaaagaaaatgaagacgaaatgctagtgatcatgacccgattcgatggtacttctatcattggcggttgtttccaacccaacaactggtcatccgaacccgatccttctctcacccatcgaatcctgtctagagccctcgaccgattcccggaactgaccaaagatggccctcttgacattgtgcgcgaatgcgttggccaccgtcctggtagagagggcggtccccgagtagaattagagaagatccccggcgttggctttgttgtccataactatggtgccgccggtgctggttaccagtcctcttacggcatggctgatgaagctgtttcttacgtcgaaagagctcttactcgtccaaacctttag

SEQ ID NO: 62 - DNA, 50 bp, Artificial Sequence
  Description of Artificial Sequence Synthetic oligonucleotide
agccatctgaccatggctaaaatcgttgttattggtgccggtgttgccgg

SEQ ID NO: 63 - DNA, 40 bp, Artificial Sequence
  Description of Artificial Sequence Synthetic oligonucleotide
attccttaaggtttaaactctaaaggtttggacgagtaag

SEQ ID NO: 64 - DNA, 42 bp, Artificial Sequence
  Description of Artificial Sequence Synthetic oligonucleotide
gattgatatcggatccatcatggctaaaatcgttgttattgg

SEQ ID NO: 65 - DNA, 38 bp, Artificial Sequence
  Description of Artificial Sequence Synthetic oligonucleotide
attccttaaggcatgctctaaaggtttggacgagtaag

SEQ ID NO: 66 - protein, 320 aa, Artificial Sequence
  Description of Artificial Sequence Synthetic polypeptide
METELDDERDGEVVVVGGGVIGLTTAVVLAERGRRVRLWTREPAERTTSVVAGGLWWPYRIEPVALAQAWALRSLDVYEELAARPGQTGVRMLEGVLGETGLDEVDGWAAARLPGLRAASAAEYAGTGLWARLPLIDMSTHLPWLRERLLAAGGTVEDRAVTDLAEADAPVVVNCTGLGARELVPDPAVRPVRGQLVVVENPGIHNWLVAADADSGEITYFLPQPGRLLLGGTAEEDAWSTEPDPEVAAAIVRRCAALRPEIAGARVLAHLVGLRPARDAVRLERGTLPDGRRLVHNYGHGGAGVTVAWGCAQEAARLAS

SEQ ID NO: 67 - protein, 320 aa, Artificial Sequence; identical to SEQ ID NO: 66
  Description of Artificial Sequence Synthetic polypeptide

SEQ ID NO: 68 - protein, 320 aa, Artificial Sequence
  Description of Artificial Sequence Synthetic polypeptide
METELDDERDGEVVVVGGGVIGLTTAVVLAERGRRVRLWTREPAERTTSVVAGGLWWPYRIEPVALAQAWALRSLDVYEELAARPGQTGVRMLEGVLGETGLDEVDGWAAARLPGLRAASAAEYAGTGLWARLPLIDMSTYLPWLRERLLAAGGTVEDRAVTDLAEADAPVVVNCTGLGARELVPDPAVRPVRGQLVVVENPGIHNWLVAADADSGETTYFLPQPGRLLLGGTAEEDAWSTEPDPEVAAAIVRRCAALRPEIAGARVLAHLVGLRPARDAVRLERGTLPDGRRLVHNYGHGGAGVTVAWGCAQEAARLAS

SEQ ID NO: 69 - protein, 320 aa, Artificial Sequence
  Description of Artificial Sequence Synthetic polypeptide
METELDDERDGEVVVVGGGVIGLTTAIVLAERGRRVRLWTREPAERTTSVVAGGLWWPYRIEPVALAQAWALRSLDVYEELAARPGQTGVRMLEGVLGETGLDEVDGWAAARLPGLRAASAAEYAGTGLWARLPLIDMSTHLPWLRERLLAAGGTVEDRAVTDLAEADAPVVVNCTGLGARELVPDPAVRPVRGQLVVVENPGIHNWLVAADADSGETTYFLPQPGRLLLGGTAEEDAWSTEPDPEVAAAIVRRCAALRPEIAGARVLAHLVGLRPARDAVRLERGTLPDGRRLVHNYGHGGAGVTVAWGCAQEAARLAS

SEQ ID NO: 70 - protein, 320 aa, Artificial Sequence; identical to SEQ ID NO: 68
  Description of Artificial Sequence Synthetic polypeptide

SEQ ID NO: 71 - protein, 320 aa, Artificial Sequence
  Description of Artificial Sequence Synthetic polypeptide
METELDDERDGEVVVVGGGVIGLTTAVVLAERGRRVRLWTREPAERTTSVVAGGLWWPYRIEPVALAQAWALRSLDVYEELAARPGQTGVRMLEGVLGETGLDEVDGWAAARLPGLRAASAAEYAGTGLWARLPLIDMSTYLPWLRERLLAAGGTVEDRAVTDLAEADAPVVVNCTGLGARELVPDPAVRPVRGQLVVVENPGIHNWLVAADADSGEITYFLPQPGRLLLGGTAEEDAWSTEPDPEVAAAIVRRCAALRPEIAGARVLAHLVGLRPARDAVRLERGTLPDGRRLVHNYGHGGAGVTVAWGCAQEAARLAS

SEQ ID NO: 72 - protein, 320 aa, Artificial Sequence
  Description of Artificial Sequence Synthetic polypeptide
METELDEERDGEVVVVGGGVIGLTTAVVLAERGRRVRLWTREPAERTTSVVAGGLWWPYRIEPVALAQAWALRSLDVYEELAARPGQTGVRMLEGVLGETGLDEVDGWAAARLPGLRAASAAEYAGTGLWARLPLIDMSTYLPWLRERLLAAGGTVEDRAVTDLAEADAPVVVNCTGLGARELVPDPAVRPVRGQLVVVENPGIHNWLVAADADSGEITYFLPQPGRLLLGGTAEEDAWSTEPDPEVAAAIVRRCAALRPEIAGARVLAHLVGLRPARDAVRLERGTLPDGRRLVHNYGHGGAGVTVAWGCAQEAARLAS

SEQ ID NO: 73 - protein, 320 aa, Artificial Sequence
  Description of Artificial Sequence Synthetic polypeptide
METELDDERDGEVVVVGGGVIGLTTAVVLAERGRRVRLWTREPAERTTSVVAGGLWWPYRIKPVALAQAWALRSLDVYEELAARPGQTGVRMLEGVLGETGLDEVDGWAAARLPGLRAASAAEYAGTGLWARLPLIDMSTYLPWLRERLLAAGGTVEDRAVTDLAEADAPVVVNCTGLGARELVPDPAVRPVRGQLVVVENPGIHNWLVAADADSGEITYFLPQPGRLLLGGTAEEDAWSTEPDPEVAAAIVRRCAALRPEIAGARVLAHLVGLRPARDAVRLERGTLPDGRRLVHNYGHGGAGVTVAWGCAQEAARLAS

SEQ ID NO: 74 - protein, 320 aa, Artificial Sequence
  Description of Artificial Sequence Synthetic polypeptide
METELDDERDGEVVVVGGGVIGLTTAVVLAERGRRVRLWTREPAERTTSVVAGGLWWPYRIEPVALARAWALRSLDVYEELAARPGQTGVRMLEGVLGETGLDEVDGWAAARLPGLRAASAAEYAGTGLWARLPLIDMSTYLPWLRERLLAAGGTVEDRAVTDLAEADAPVVVNCTGLGARELVPDPAVRPVRGQLVVVENPGIHNWLVAADADSGEITYFLPQPGRLLLGGTAEEDAWSTEPDPEVAAAIVRRCAALRPEIAGARVLAHLVGLRPARDAVRLERGTLPDGRRLVHNYGHGGAGVTVAWGCAQEAARLAS

SEQ ID NO: 75 - protein, 320 aa, Artificial Sequence
  Description of Artificial Sequence Synthetic polypeptide
METELDDERDGEVVVVGGGVIGLTTAVVLAERGRRVRLWTREPAERTTSAVAGGLWWPYRIEPVALAQAWALRSLDVYEELAARPGQTGVRMLEGVLGETGLDEVDGWAAARLPGLRAASAAEYAGTGLWARLPLIDMSTYLPWLRERLLAAGGTVEDRAVTDLAEADAPVVVNCTGLGARELVPDPAVRPVRGQLVVVENPGIHNWLVAADADSGEITYFLPQPGRLLLGGTAEEDAWSTEPDPEVAAAIVRRCAALRPEIAGARVLAHLVGLRPARDAVRLERGTLPDGRRLVHNYGHGGAGVTVAWGCAQEAARLAS

SEQ ID NO: 76 - protein, 320 aa, Artificial Sequence
  Description of Artificial Sequence Synthetic polypeptide
METELDDERDGEVVVVGGGVIGLTTAVVLAERGRRVRLWTREPAERTTSVVAGGLWWPYRIEPVALAQAWALRSLDVYEELAARPGQTGVRMVEGVLGETGLDEVDGWAAARLPGLRAASAAEYAGTGLWARLPLIDMSTYLPWLRERLLAAGGTVEDRAVTDLAEADAPVVVNCTGLGARELVPDPAVRPVRGQLVVVENPGIHNWLVAADADSGEITYFLPQPGRLLLGGTAEEDAWSTEPDPEVAAAIVRRCAALRPEIAGARVLAHLVGLRPARDAVRLERGTLPDGRRLVHNYGHGGAGVTVAWGCAQEAARLAS

SEQ ID NO: 77 - protein, 320 aa, Artificial Sequence
  Description of Artificial Sequence Synthetic polypeptide
METELDDERDGEVVVVGGGVIGLTTAVVLAERGRRVRLWTREPAERTTSVVAGGLWWPYRIEPVALAQAWALRSLDVYEELAARPGQTGVRMLEGVLGGTGLDEVDGWAAARLPGLRAASAAEYAGTGLWARLPLIDMSTYLPWLRERLLAAGGTVEDRAVTDLAEADAPVVVNCTGLGARELVPDPAVRPVRGQLVVVENPGIHNWLVAADADSGEITYFLPQPGRLLLGGTAEEDAWSTEPDPEVAAAIVRRCAALRPEIAGARVLAHLVGLRPARDAVRLERGTLPDGRRLVHNYGHGGAGVTVAWGCAQEAARLAS

SEQ ID NO: 78 - protein, 320 aa, Artificial Sequence
  Description of Artificial Sequence Synthetic polypeptide
METELDDERDGEVVVVGGGVIGLTTAVVLAERGRRVRLWTREPAERTTSVVAGGLWWPYRIEPVALAQAWALRSLDVYEELAARPGQTGVRMLEGVLGETGLDEVDGWAAARLPGLRAASAAEYAGTGLWARLPLIDMSTYLPWLRERLLAAGGTVEDRAVTDLAEADAPVVVNCTGLGARELVPDPAVRPVRGQLVVVENPGIHNWLVATDADSGEITYFLPQPGRLLLGGTAEEDAWSTEPDPEVAAAIVRRCAALRPEIAGARVLAHLVGLRPARDAVRLERGTLPDGRRLVHNYGHGGAGVTVAWGCAQEAARLAS

SEQ ID NO: 79 - protein, 320 aa, Artificial Sequence
  Description of Artificial Sequence Synthetic polypeptide
METELDDERDGEVVVVGGGVIGLTTAVVLAERGRRVRLWTREPAERTTSVVAGGLWWPYRIEPVALAQAWALRSLDVYEELAARPGQTGVRMLEGVLGETGLDEVDGRAAARLPGLRAASAAEYAGTGLWARLPLIDMSTYLPWLRERLLAAGGTVEDRAVTDLAEADAPVVVNCTGLGARELVPDPAVRPVRGQLVVVENPGIHNWLVAADADSGEITYFLPQPGRLLLGGTAEEDAWSTEPDPEVAAAIVRRCAALRPEIAGARVLAHLVGLRPARDAVRLERGTLPDGRRLVHNYGHGGAGVTVAWGCAQEAARLAS

SEQ ID NO: 80 - protein, 320 aa, Artificial Sequence
  Description of Artificial Sequence Synthetic polypeptide
METELDDERDGEVVVVGSGVIGLTTAVVLAERGRRVRLWTREPAERTTSAVAGGLWWPYRIEPVALAQAWALRSLDVYEELAARPGQTGVRMLEGVLGETGLDEVDGWAAARLPGLRAASAAEYAGTGLWARLPLIDMSTYLPWLRERLLAAGGTVEDRAVTDLAEADAPVVVNCTGLGARELVPDPAVRPVRGQLVVVENPGIHNWLVAADADSGEITYFLPQPGRLLLGGTAEEDAWSTEPDPEVAAAIVRRCAALRPEIAGARVLAHLVGLRPARDAVRLERGTLPDGRRLVHNYGHGGAGVTVAWGCAQEAARLAS

SEQ ID NO: 81 - protein, 320 aa, Artificial Sequence
  Description of Artificial Sequence Synthetic polypeptide
METELDDERDGEVVVVGGGVIGLTTAVVLAERGRRVRLWTREPAERTTSAVAGGLWWPYLIEPVALAQAWALRSLDVYEELAARPGQTGVRMLEGVLGETGLDEVDGWAAARLPGLRAASAAEYAGTGLWARLPLIDMSTYLPWLRERLLAAGGTVEDRAVTDLAEADAPVVVNCTGLGARELVPDPAVRPVRGQLVVVENPGIHNWLVAADADSGEITYFLPQPGRLLLGGTAEEDAWSTEPDPEVAAAIVRRCAALRPEIAGARVLAHLVGLRPARDAVRLERGTLPDGRRLVHNYGHGGAGVTVAWGCAQEAARLAS

SEQ ID NO: 82 - protein, 320 aa, Artificial Sequence
  Description of Artificial Sequence Synthetic polypeptide
METELDDERDGEVVVVGGGVIGLTTAVVLAERGRRVRLWTREPAERTTSAVAGGLWWPYRIEPVALAQAWALRSLDVYEELAARPGQTGVRMLEGVLGETGLDEVDGWAAARLPGLRAASAAEYAGTGLWARLPLIDMSTYLPWLRERLLAAGGTVEDRAVTDLAEADAPVVVNCTGLGARELVPDPAVRPVRGQLVVVENPGIHNWLVAADADSSEITYFLPQPGRLLLGGTAEEDAWSTEPDPEVAAAIVRRCAALRPEIAGARVLAHLVGLRPARDAVRLERGTLPDGRRLVHNYGHGGAGVTVAWGCAQEAARLAS

SEQ ID NO: 83 - protein, 320 aa, Artificial Sequence
  Description of Artificial Sequence Synthetic polypeptide
METELDDERDGEVVVVGGGVIGLTTAVVLAERGRRVRLWTREPAERTTAAVAGGLWWPYRIEPVALAQAWALRSLDVYEELAARPGQTGVRMLEGVLGETGLDEVDGWAAARLPGLRAASAAEYAGTGLWARLPLIDMSTYLPWLRERLLAAGGTVEDRAVTDLAEADAPVVVNCTGLGARELVPDPAVRPVRGQLVVVENPGIHNWLVTADADSGEITYFLPQPGRLLLGGTAEEDAWSTEPDPEVAAAIVRRCAALRPEIAGARVLAHLVGLRPARDAVRLERGTLPDGRRLVHNYGHGGAGVTVAWGCAQEAARLAS

SEQ ID NO: 84 - protein, 320 aa, Artificial Sequence
  Description of Artificial Sequence Synthetic polypeptide
METELDDERDGEVVVVGGGVIGLTTAVVLAERGRRVRLWTREPAERTTSAVAGGLWWPYRIEPVALAQAWALRSLDVYEELAARPGQTGVRMLEGVLGEMGLDEVDGWAAARLPGLRAASAAEYAGTGLWARLPLIDMSTYLPWLRERLLAAGGTVEDRAVTDLAEADAPVVVNCTGLGARELVPDPAVRPVRGQLVVVENPGIHNWLVTADADSGEITYFLPQPGRLLLGGTAEEDAWSTEPDPEVAAAIVRRCAALRPEIAGARVLAHLVGLRPARDAVRLERGTLPDGRRLVHNYGHGGAGVTVAWGCAQEAARLAS

SEQ ID NO: 85 - protein, 320 aa, Artificial Sequence
  Description of Artificial Sequence Synthetic polypeptide
METELDDERDGEVVVVGGGVIGLTTAVVLAERGRRVRLWTREPAERTTSAVAGGLWWPYRIEPVALAQAWALRSLDVYEELAARPGQTGVRMLEGVLGETGLDEVDGWAAARLPGLRAASAAEYAGTGLWARLPLIDMSTYLPWLRERLLAAGGTVEDRAVTDLAEADAPVVVNCTGLGARELVPDPAVRPVRGQLVVVENPGIHNWLVATDADSGEITYFLPQPGRLLLGGTAEEDAWSTEPDPEVAAAIVRRCAALRPEIAGARVLAHLVGLRPARDAVRLERGTLPDGRRLVHNYGHGGAGVTVAWGCAQEAARLAS

SEQ ID NO: 86 - protein, 320 aa, Artificial Sequence
  Description of Artificial Sequence Synthetic polypeptide
METELDDERDGEVVVVGGGVIGLTTAVVLAERGRRVRLWTREPAERTTSAVAGGLWWPYRIEPVALAQAWALRSLDVYEELAARPGQTGVRMVEGVLGETGLDEVDGWAAARLPGLRAASAAEYAGTGLWARLPLIDMSTYLPWLRERLLAAGGTVEDRAVTDLAEADAPVVVNCTGLGARELVPDPAVRPVRGQLVVVENPGIHNWLVAADADSGEITYFLPQPGRLLLGGTAEEDAWSTEPDPEVAAAIVRRCAALRPEIAGARVLAHLVGLRPARDAVRLERGTLPDGRRLVHNYGHGGAGVTVAWGCAQEAARLAS

SEQ ID NO: 87 - protein, 320 aa, Artificial Sequence
  Description of Artificial Sequence Synthetic polypeptide
METELDDERDGEVVVVGGGVIGLTTAVVLAERGRRVRLWTREPAERTTSAVAGGLWWPYRIEPVALAQAWALRSLDVYEELAARPGQTGVRMLEGVLGETGLDEVDGWAAARLPGLRAASAAEYAGTGLWARLPLIDMSTYLPWLRERLLAAGGTVEYRAVTDLAEADAPVVVNCTGLGARELVPDPAVRPVRGQLVVVENPGIHNWLVAADADSGEITYFLPQPGRLLLGGTAEEDAWSTEPDPEVAAAIVRRCAALRPEIAGARVLAHLVGLRPARDAVRLERGTLPDGRRLVHNYGHGGAGVTVAWGCAQEAARLAS

SEQ ID NO: 88 - protein, 320 aa, Artificial Sequence
  Description of Artificial Sequence Synthetic polypeptide
METELDDERDGEVVVVGGGVIGLTTAVVLAERGRRVRLWTREPAERTTSAVAGGLWWPYRIEPVALAQAWALRSLDVYEELAARPGQTGVRMLEGVLGETGLDEVDGWAAGRLPGLRAASAAEYAGTGLWARLPLIDMSTYLPWLRERLLAAGGTVEDRAVTDLAEADAPVVVNCTGLGARELVPDPAVRPVRGQLVVVENPGIHNWLVAADADSGEITYFLPQPGRLLLGGTAEEDAWSTEPDPEVAAAIVRRCAALRPEIAGARVLAHLVGLRPARDAVRLERGTLPDGRRLVHNYGHGGAGVTVAWGCAQEAARLAS

SEQ ID NO: 89 - protein, 320 aa, Artificial Sequence
  Description of Artificial Sequence Synthetic polypeptide
METELDDERDGEVVVVGGGVIGLTTAVVLAERGRRVRLWTREPAERTTSAVAGGLWWPYRIEPVALAQAWALRSLDVYEELAARPGQTGVRMLEGVLGETGLDEVDGWAAARLPGLRAASAAEYAGTGVWARLPLIDMSTYLPWLRERLLAAGGTVEDRAVTDLAEADAPVVVNCTGLGARELVPDPAVRPVRGQLVVVENPGIHNWLVAADADSGEITYFLPQPGRLLLGGTAEEDAWSTEPDPEVAAAIVRRCAALRPEIAGARVLAHLVGLRPARDAVRLERGTLPDGRRLVHNYGHGGAGVTVAWGCAQEAARLAS

SEQ ID NO: 90 - protein, 320 aa, Artificial Sequence
  Description of Artificial Sequence Synthetic polypeptide
METELDDERDGEVVVVGGGVIGLTTAVVLAERGRRVRLWTREPAERTTSAVAGGLWWPYRIEPVALAQAWALRSLDVYEELAARPGQTGVRMLEGVLGETSLDEVVGWAAARLPGLRAASAAEYAGTGLWARLPLIDMSTYLPWLRERLLAAGGTVEDRAVTDLAEADAPVVVNCTGLGARELVPDPAVRPVRGQLVVVENPGIHNWLVAADADSGEITYFLPQPGRLLLGGTAEEDAWSTEPDPEVAAAIVRRCAALRPEIAGARVLAHLVGLRPARDAVRLERGTLPDGRRLVHNYGHGGAGVTVAWGCAQEAARLAS

SEQ ID NO: 91 - protein, 320 aa, Artificial Sequence
  Description of Artificial Sequence Synthetic polypeptide
METELDDVRDGEVVVVGSGVIGLTTAVVLAERGRRVRLWTREPAERTTSAVAGGLWWPYRIEPVALAQAWALRSLDVYEELAARPGQTGVRMLEGVLGETGLDEVDGWAAARLPGLRAASAAEYAGTGLWARLPLIDMSTYLPWLRERLLAAGGTVEDRAVTDLAEADAPVVVNCTGLGARELVPDPAVRPVRGQLVVVENPGIHNWLVAADADSGEITYFLPQPGRLLLGGTAEEDAWSTEPDPEVAAAIVRRCAALRPEIAGARVLAHLVGLRPARDAVRLERGTLPDGRRLVHNYGHGGAGVTVAWGCAQEAARLAS

SEQ ID NO: 92 - protein, 320 aa, Artificial Sequence
  Description of Artificial Sequence Synthetic polypeptide
METELDDERDGEVVVVGGGVIGLTTAVVLAERGRRVRLWTREPAERTTSAVAGGLWWPYRIEPVALAQAWALRSLDVYEELAARPGQTGVRMLEGVLGETGLDEVDGWAAARLPGLRAASAAEYAGTGLWARLPLIDMSTYLPWLRERLLAAGGTVEDRAVTDLAEADAPVVVNCTGLGARELVPDPAVRPVRGQLVVVENPGIHNWLVAADADSGEITYYLPQPGRLLLGGTAEEDAWSTEPDPEVAAAIVRRCAALRPEIAGARVLAHLVGLRPARDAVRLERGTLPDGRRLVHNYGHGGAGVTVAWGCAQEAARLAS

SEQ ID NO: 93 - protein, 320 aa, Artificial Sequence
  Description of Artificial Sequence Synthetic polypeptide
METELDDERDGEVVVVGGGVIGLTTAVVLAERGRRVRLWTREPAERTTSAVAGGLWWPYRIEPVALAQAWALRSLDVYEELAARPGQTGVRMLEGVLGETSLDEVDGWAAARIPGLRAASAAEYAGTGLWARLPLIDMSTYLPWLRERLLAAGGTVEDRAVTDLAEADAPVVVNCTGLGARELVPDPAVRPVRGQLVVVENPGIHNWLVAADADSGEITYFLPQPGRLLLGGTAEEDAWSTEPDPEVAAAIVRRCAALRPEIAGARVLAHLVGLRPARDAVRLERGTLPDGRRLVHNYGHGGAGVTVAWGCAQEAARLAS

SEQ ID NO: 94 - protein, 320 aa, Artificial Sequence
  Description of Artificial Sequence Synthetic polypeptide
METELDDERDGEVVVVGGGVIGLTTAVVLAERGRRVRLWTREPAERTTSAVAGGLWWPYRIEPVALAQAWALRSLDVYEELAARPGQTGVRMLEGVLGETGLDEVDGWAAARLPGLRAASAAEYAGTGLWARLPLIDMSTYLPWLRERLLAAGGTVEDRAVTDLAEADAPVVVNCTGLGARELVPDPAVRPVRGQLVVVENPGIHNWLVTADADSGEITYFLPQPGRLLLGGTAEEDAWSTEPDPEVAAAIVRRCAALRPEIAGARVLAHLVGLRPARDAVRLERGTLPDGRRLVHNYGHGGAGVTVAWGCAQEAARLAS

SEQ ID NO: 95 - protein, 348 aa, Artificial Sequence
  Description of Artificial Sequence Synthetic polypeptide
MTKENKPRDIVIVGAGVIGLTTAWILSDLGLAPRIKVIAKYTPEDRSVEYTSPWAGANFCSISATDDNALRWDKITYHRFAYLAKTRPEAGIRFADLRELWEYEPKHDKIRSWNTYVRDFKVIPEKDLPGECIYGHKATTFLINAPHYLNYMYKLLIEAGVEFEKKELSHIKETVEETPEASVVFNCTGLWASKLGGVEDPDVYPTRGHVVLVKAPHVTETRILNGKNSDTYIIPRPLNGGVICGGFMQPGNWDREIHPEDTLDILKRTSALMPELFHGKGPEGAEIIQECVGFRPSRKGGARVELDVVPGTSVPLVHDYGASGTGYQAGYGMALDSVMLALPKIKLA

SEQ ID NO: 96 - protein, 348 aa, Artificial Sequence
  Description of Artificial Sequence Synthetic polypeptide
MTKENKPRDIVIVGAGVIGLTTAWILSDLGLAPRIKVIAKYTPEDRSVEYTSPWAGANFCSISATDDNALRWDKITYHRFAYLAKTRPEAGIRFADLRELWEYEPKHDKIRSWNTYVRDFKVIPEKDLPGECIYGHKATTFLINAPHYLNYMYKLLIEAGVEFEKKELSHIKETVEETPEASVVFNCTGLWASKLGGVEDPDVYPTRGHVVLVKAPHVTETRILNGKNSDTGIIPRPLNGGVICGGFMQPGNWDREIHPEDTLDILKRTSALMPELFHGKGPEGAEIIQECVGFRPSRKGGARVELDVVPGTSVPLVHDYGASGTGYQAGYGMALDSVMLALPKIKLA

SEQ ID NO: 97 - protein, 348 aa, Artificial Sequence
  Description of Artificial Sequence Synthetic polypeptide
MTKENKPRDIVIVGAGVIGLTTAWILSDLGLAPRIKVIAKYTPEDRSVEYTSPWAGANFCSISATDDNALRWDKITYHRFAYLAKTRPEAGIRFADLRELWEYEPKHDKIRSWNTYVRDFKVIPEKDLPGECIYGHKATTFLINAPHYLNYMYKLLIEAGVEFEKKELSHIKETVEETPEASVVFNCTGLWASKLGGVEDPDVYPTRGHVVLVKAPHVTETRILNGKNSDTHIIPRPLNGGVICGGFMQPGNWDREIHPEDTLDILKRTSALMPELFHGKGPEGAEIIQECVGFRPSRKGGARVELDVVPGTSVPLVHDYGASGTGYQAGYGMALDSVMLALPKIKLA

SEQ ID NO: 98 - protein, 348 aa, Artificial Sequence
  Description of Artificial Sequence Synthetic polypeptide
MTKENKPRDIVIVGAGVIGLTTAWILSDLGLAPRIKVIAKYTPEDRSVEYTSPWAGANFCSISATDDNALRWDKITYHRFAYLAKTRPEAGIRFADLRELWEYEPKHDKIRSWNTYVRDFKVIPEKDLPGECIYGHKATTFLINAPHYLNYMYKLLIEAGVEFEKKELSHIKETVEETPEASVVFNCTGLWASKLGGVEDPDVYPTRGHVVLVKAPHVTETRILNGKNSDTIIIPRPLNGGVICGGFMQPGNWDREIHPEDTLDILKRTSALMPELFHGKGPEGAEIIQECVGFRPSRKGGARVELDVVPGTSVPLVHDYGASGTGYQAGYGMALDSVMLALPKIKLA

SEQ ID NO: 99 - protein, 348 aa, Artificial Sequence
  Description of Artificial Sequence Synthetic polypeptide
MTKENKPRDIVIVGAGVIGLTTAWILSDLGLAPRIKVIAKYTPEDRSVEYTSPWAGANFCSISATDDNALRWDKITYHRFAYLAKTRPEAGIRFADLRELWEYEPKHDKIRSWNTYVRDFKVIPEKDLPGECIYGHKATTFLINAPHYLNYMYKLLIEAGVEFEKKELSHIKETVEETPEASVVFNCTGLWASKLGGVEDPDVYPTRGHVVLVKAPHVTETRILNGKNSDTSIIPRPLNGGVICGGFMQPGNWDREIHPEDTLDILKRTSALMPELFHGKGPEGAEIIQECVGFRPSRKGGARVELDVVPGTSVPLVHDYGASGTGYQAGYGMALDSVMLALPKIKLA

SEQ ID NO: 100 - protein, 348 aa, Artificial Sequence
  Description of Artificial Sequence Synthetic polypeptide
MTKENKPRDIVIVGAGVIGLTTAWILSDLGLAPRIKVIAKYTPEDRSVEYTSPWAGANFCSISATDDNALRWDKITYHRFAYLAKTRPEAGIRFADLRELWEYEPKHDKIRSWNTYVRDFKVIPEKDLPGECIYGHKATTFLINAPHYLNYMYKLLIEAGVEFEKKELSHIKETVEETPEASVVFNCTGLWASKLGGVEDPDVYPTRGHVVLVKAPHVTETRILNGKNSDTMIIPRPLNGGVICGGFMQPGNWDREIHPEDTLDILKRTSALMPELFHGKGPEGAEIIQECVGFRPSRKGGARVELDVVPGTSVPLVHDYGASGTGYQAGYGMALDSVMLALPKIKLA

SEQ ID NO: 101 - protein, 348 aa, Artificial Sequence
  Description of Artificial Sequence Synthetic polypeptide
MTKENKPRDIVIVGAGVIGLTTAWILSDLGLAPRIKVIAKYTPEDRSVEYTSPWAGANFCSISATDDNALRWDKITYHRFAYLAKTRPEAGIRFADLRELWEYEPKHDKIRSWNTYVRDFKVIPEKDLPGECIYGHKATTFLINAPHYLNYMYKLLIEAGVEFEKKELSHIKETVEETPEASVVFNCTGLWASKLGGVEDPDVYPTRGHVVLVKAPHVTETRILNGKNSDTQIIPRPLNGGVICGGFMQPGNWDREIHPEDTLDILKRTSALMPELFHGKGPEGAEIIQECVGFRPSRKGGARVELDVVPGTSVPLVHDYGASGTGYQAGYGMALDSVMLALPKIKLA

SEQ ID NO: 102 - protein, 348 aa, Artificial Sequence
  Description of Artificial Sequence Synthetic polypeptide
MTKENKPRDIVIVGAGVIGLTTAWILSDLGLAPRIKVIAKYTPEDRSVEYTSPWAGANFCSISATDDNALRWDKITYHRFAYLAKTRPEAGIRFADLRELWEYEPKHDKIRSWNTYVRDFKVIPEKDLPGECIYGHKATTFLINAPHYLNYMYKLLIEAGVEFEKKELSHIKETVEETPEASVVFNCTGLWASKLGGVEDPDVYPTRGHVVLVKAPHVTETRILNGKNSDTCIIPRPLNGGVICGGFMQPGNWDREIHPEDTLDILKRTSALMPELFHGKGPEGAEIIQECVGFRPSRKGGARVELDVVPGTSVPLVHDYGASGTGYQAGYGMALDSVMLALPKIKLA

SEQ ID NO: 103 - protein, 348 aa, Artificial Sequence
  Description of Artificial Sequence Synthetic polypeptide
MTKENKPRDIVIVGAGVIGLTTAWILSDLGLAPRIKVIAKYTPEDRSVEYTSPWAGANFCSISATDDNALRWDKITYHRFAYLAKTRPEAGIRFADLRELWEYEPKHDKIRSWNTYVRDFKVIPEKDLPGECIYGHKATTFLINAPHYLNYMYKLLIEAGVEFEKKELSHIKETVEETPEASVVFNCTGLWASKLGGVEDPDVYPTRGHVVLVKAPHVTETRILNGKNSDTAIIPRPLNGGVICGGFMQPGNWDREIHPEDTLDILKRTSALMPELFHGKGPEGAEIIQECVGFRPSRKGGARVELDVVPGTSVPLVHDYGASGTGYQAGYGMALDSVMLALPKIKLA

SEQ ID NO: 104 - protein, 356 aa, Artificial Sequence
  Description of Artificial Sequence Synthetic polypeptide
MAKIVVIGAGVAGLTTALQLLRKGHEVTIVSEFTPGDLSIGYTSPWAGANWLTFYDGGKLADYDAVSYPILRELARSSPEAGIRLINQRSHVLKRDLPKLEGAMSAICQRNPWFKNTVDSFEIIEDRSRIVHDDVAYLVEFASVCIHTGVYLNWLMSQCLSLGATVVKRRVNHIKDANLLHSSGSRPDVIVNCSGLFARFLGGVEDKKMYPIRGQVVLVRNSLPFMASFSSTPEKENEDEALYIMTRFDGTSIIGGCFQPNNWSSEPDPSLTHRILSRALDRFPELTKDGPLDIVRECVGHRPGREGGPRVELEKIPGVGFVVHNYGAAGAGYQSSYGMADEAVSYVERALTRPNL

SEQ ID NO: 105 - protein, 356 aa, Artificial Sequence
  Description of Artificial Sequence Synthetic polypeptide
MAKIVVIGAGVAGLTTALQLLRKGHEVTIVSEFTPGDLSIGYTSPWAGANWLTFYDGGKLADYDAVSYPILRELARSSPEAGIRLINQRSHVLKRDLPKLEGAMSAICQRNPWFKNTVDSFEIIEDRSRIVHDDVAYLVEFASVCIHTGVYLNWLMSQCLSLGATVVKRRVNHIKDANLLHSSGSRPDVIVNCSGLFARFLGGVEDKKMYPIRGQVVLVRNSLPFMASFSSTPEKENEDEALHIMTRFDGTSIIGGCFQPNNWSSEPDPSLTHRILSRALDRFPELTKDGPLDIVRECVGHRPGREGGPRVELEKIPGVGFVVHNYGAAGAGYQSSYGMADEAVSYVERALTRPNL

SEQ ID NO: 106 - protein, 356 aa, Artificial Sequence
  Description of Artificial Sequence Synthetic polypeptide
MAKIVVIGAGVAGLTTALQLLRKGHEVTIVSEFTPGDLSIGYTSPWAGANWLTFYDGGKLADYDAVSYPILRELARSSPEAGIRLINQRSHVLKRDLPKLEGAMSAICQRNPWFKNTVDSFEIIEDRSRIVHDDVAYLVEFASVCIHTGVYLNWLMSQCLSLGATVVKRRVNHIKDANLLHSSGSRPDVIVNCSGLFARFLGGVEDKKMYPIRGQVVLVRNSLPFMASFSSTPEKENEDEHLVIMTRFDGTSIIGGCFQPNNWSSEPDPSLTHRILSRALDRFPELTKDGPLDIVRECVGHRPGREGGPRVELEKIPGVGFVVHNYGAAGAGYQSSYGMADEAVSYVERALTRPNL

SEQ ID NO: 107 - protein, 356 aa, Artificial Sequence
  Description of Artificial Sequence Synthetic polypeptide
MAKIVVIGAGVAGLTTALQLLRKGHEVTIVSEFTPGDLSIGYTSPWAGANWLTFYDGGKLADYDAVSYPILRELARSSPEAGIRLINQRSHVLKRDLPKLEGAMSAICQRNPWFKNTVDSFEIIEDRSRIVHDDVAYLVEFASVCIHTGVYLNWLMSQCLSLGATVVKRRVNHIKDANLLHSSGSRPDVIVNCSGLFARFLGGVEDKKMYPIRGQVVLVRNSLPFMASFSSTPEKENEDEMLVIMTRFDGTSIIGGCFQPNNWSSEPDPSLTHRILSRALDRFPELTKDGPLDIVRECVGHRPGREGGPRVELEKIPGVGFVVHNYGAAGAGYQSSYGMADEAVSYVERALTRPNL

SEQ ID NO: 108 - protein, 356 aa, Artificial Sequence
  Description of Artificial Sequence Synthetic polypeptide
MAKIVVIGAGVAGLTTALQLLRKGHEVTIVSEFTPGDLSIGYTSPWAGANWLTFYDGGKLADYDAVSYPILRELARSSPEAGIRLINQRSHVLKRDLPKLEGAMSAICQRNPWFKNTVDSFEIIEDRSRIVHDDVAYLVEFASVCIHPGVYLNWLLSQCLSLGATVVKRRVNHIKDANLLHSSGSRPDVIVNCSGLFARFLGGVEDKKLYPIRGQVVLVRNSLPFMASFSSTPEKENEDEMLVIMTRFDGTSIIGGCFQPNNWSSEPDPSLTHRILSRALDRFPELTKDGPLDIVRECVGHRPGREGGPRVELEKIPGVGFVVHNYGAAGAGYQSSYGMADEAVSYVERALTRPNL

SEQ ID NO: 109 - protein, 356 aa, Artificial Sequence
  Description of Artificial Sequence Synthetic polypeptide
MAKIVVIGAGVAGLTTALQLLRKGHEVTIVSEFTPGDLSIRYTSPWAGANWLTFYDGGKLADYDAVSYPILRELARSSPEAGIRLINQRSHVLKRDLPKLEGAMSTICQRNPWFKNTVDSFEIIEDRSRIVHDDVAYLVEFASVCIHPGVYLNWLLSQCLSLGATVVKRRVNHIKDANLLHSSGSRPDVIVNCSGLFARFLGGVEDKKLYPIRGQVVLVRNSLPFMASFSSTPEKENEDEMLVIMTRFDGTSIIGGCFQPNNWSSEPDPSLTHRILSRALDRFPELTKDGPLDIVRECVGHRPGREGGPRVELEKIPGVGFVVHNYGAAGAGYQSSYGMADEAVSYVERALTRPNL

SEQ ID NO: 110 - protein, 356 aa, Artificial Sequence
  Description of Artificial Sequence Synthetic polypeptide
MAKIVVIGAGVAGLTTALQLLRKGHEVTIVSEFTPGDLSIRYTSPWAGANWLTFSDGGKLADYDAVSYPILRELARSSPEAGIRLINQRSHVLKRDLPKLEGAMSTICQRNPWFKNTVDSFEIIEDRSRIVHDDVAYLVEFASVCIHPGVYLNWLLSQCLSLGATVVKRRVNHIKDANLLHSSGSRPDVIVNCSGLFARFLGGVEDKKLYPIRGQVVLVRNSLPFMASFSSTPEKENEDEMLVIMTRFDGTSIIGGCFQPNNWSSEPDPSLTHRILSRALDRFPELTKDGPLDIVRECVGHRPGREGGPRVELEKIPGVGFVVHNYGAAGAGYQSSYGMADEAVSYVERALTRPNL

SEQ ID NO: 111 - protein, 356 aa, Artificial Sequence
  Description of Artificial Sequence Synthetic polypeptide
MAKIVVIGAGVAGLTTALQLLRKGHEVTIVSEFTPGDLSIRYTSPWAGANWLTFSEGGKLADYDAVSYPILRELARSSPEAGIRLINQRSHVLKRDLPKLEGAMSTICQRNPWFKNTVDSFEIIEDRSRIVHDDVAYLVEFASVCIHPGVYLNWLLSQCLSLGATVVKRRVNHIKDANLLHSSGSRPDVIVNCSGLFARFLGGVEDKKLYPIRGQVVLVRNSLPFMASFSSTPEKENEDEMLVIMTRFDGTSIIGGCFQPNNWSSEPDPSLTHRILSRALDRFPELTKDGPLDIVRECVGHRPGREGGPRVELEKIPGVGFVVHNYGAAGAGYQSSYGMADEAVSYVERALTRPNL

SEQ ID NO: 112 - protein, 356 aa, Artificial Sequence
  Description of Artificial Sequence Synthetic polypeptide
MAKIVVIGAGVAGLTTALQLLRKGHEVTIVSEFTPGDLSIRYTSPWAGANWLTFSEGGKLADYDAVSYPILRELARSSPEAGIRLINQRSHVLKRDLPKLEGPMSTICQRNPWFKNTVDSFEIIEDRSRIVHDDVAYLVEFASVCIHPGVYLNWLLSQCLSLGATVVKRRVNHIKDANLLHSSGSRPDVIVNCSGLFARFLGGVEDKKLYPIRGQVVLVRNSLPFMASFSSTPEKENEDEMLVIMTRFDGTSIIGGCFQPNNWSSEPDPSLTHRILSRALDRFPELTKDGPLDIVRECVGHRPGREGGPRVELEKIPGVGFVVHNYGAAGAGYQSSYGMADEAVSYVERALTRPNL

SEQ ID NO: 113 - protein, 356 aa, Artificial Sequence
  Description of Artificial Sequence Synthetic polypeptide
MAKIVVIGAGVAGLTTALQLLRKGHEVTIVSEFTPGDLSIRYTSPWAGANWLTFYDGGKLADYDAVSYPILRELARSSPEAGIRLINQRSHVLKRDLPKLEGAMSTICQRNPWFKNTVDSFEIIEDRSRIVHDDVAYLVEFASVCIHPGVYLNWLLSQCLSLGATVVKRRVNHIKDANLLHSSGSRPDVIVNCSGLFARFLGGVEDKKLYPIRGQVVLVRNSLPFMASFSSTPEKENEDEALYIMTRFDGTSIIGGCFQPNNWSSEPDPSLTHRILSRALDRFPELTKDGPLDIVRECVGHRPGREGGPRVELEKIPGVGFVVHNYGAAGAGYQSSYGMADEAVSYVERALTRPNL

SEQ ID NO: 114 - protein, 356 aa, Artificial Sequence
  Description of Artificial Sequence Synthetic polypeptide
MAKIVVIGAGVAGLTTALQLLRKGHEVTIVSEFTPGDLSIRYTSPWAGANWLTFYDGGKLADYDAVSYPILRELARSSPEAGIRLINQRSHVLKRDLPKLEGAMSTICQRNPWFKNTVDSFEIIEDRSRIVHDDVAYLVEFASVCIHPGVYLNWLLSQCLSLGATVVKRRVNHIKDANLLHSSGSRPDVIVNCSGLFARFLGGVEDKKLYPIRGQVVLVRNSLPFMAWFSSTPEKENEDEALYIMTRFDGTSIIGGCFQPNNWSSEPDPSLTHRILSRALDRFPELTKDGPLDIVRECVGHRPGREGGPRVELEKIPGVGFVVHNYGAAGAGYQSSYGMADEAVSYVERALTRPNL

SEQ ID NO: 115 - protein, 356 aa, Artificial Sequence
  Description of Artificial Sequence Synthetic polypeptide
MAKIVVIGAGVAGLTTALQLLRKGHEVTIVSEFTPGDLSIRYTSPWAGANWLTFYDGGKLADYDAVSYPILRELARSSPEAGIRLINQRSHVLKRDLPKLEGAMSTICQRNPWFKNTVDSFEIIEDRSRIVHDDVAYLVEFASVCIHPGVYLNWLLSQCLSLGATVVKRRVNHIKDANLLHSSGSRPDVIVNCSGLFARFLGGVEDKKLYPIRGQVVLVRNSLPFMAYFSSTPEKENEDEALYIMTRFDGTSIIGGCFQPNNWSSEPDPSLTHRILSRALDRFPELTKDGPLDIVRECVGHRPGREGGPRVELEKIPGVGFVVHNYGAAGAGYQSSYGMADEAVSYVERALTRPNL

SEQ ID NO: 116 - protein, 356 aa, Artificial Sequence
  Description of Artificial Sequence Synthetic polypeptide
MAKIVVIGAGVAGLTTALQLLRKGHEVTIVSEFTPGDLSIRYTSPWAGANWLTFYDGGKLADYDAVSYPILRELARSSPEAGIRLINQRSHVLKRDLPKLEGAMSTICQRNPWFKNTVDSFEIIEDRSRIVHDDVAYLVEFASVCIHPGVYLNWLLSQCLSLGATVVKRRVNHIKDANLLHSSGSRPDVIVNCSGLFARFLGGVEDKKLYPIRGQVVLVRNSLPFMALFSSTPEKENEDEALYIMTRFDGTSIIGGCFQPNNWSSEPDPSLTHRILSRALDRFPELTKDGPLDIVRECVGHRPGREGGPRVELEKIPGVGFVVHNYGAAGAGYQSSYGMADEAVSYVERALTRPNL

SEQ ID NO: 117 - protein, 356 aa, Artificial Sequence
  Description of Artificial Sequence Synthetic polypeptide
MAKIVVIGAGVAGLTTALQLLRKGHEVTIVSEFTPGDLSIRYTSPWAGANWLTFSEGGKLADYDAVSYPILRELARSSPEAGIRLINQRSHVLKRDLPKLEGPMSTICQRNPWFKNTVDSFEIIEDRSRIVHDDVAYLVEFASVCIHPGVYLNWLLSQCLSLGATVVKRRVNHIKDANLLHSSGSRPDVIVNCSGLFARFLGGVEDKKLYPIRGQVVLVRNSLPFMAIFSSTPEKENEDEMLVIMTRFDGTSIIGGCFQPNNWSSEPDPSLTHRILSRALDRFPELTKDGPLDIVRECVGHRPGREGGPRVELEKIPGVGFVVHNYGAAGAGYQSSYGMADEAVSYVERALTRPNL

SEQ ID NO: 118 - protein, 356 aa, Artificial Sequence
  Description of Artificial Sequence Synthetic polypeptide
MAKIVVIGAGVAGLTTALQLLRKGHEVTIVSEFTPGDLSIRYTSPWAGANWLTFSEGGKLADYDAVSYPILRELARSSPEAGIRLINQRSHVLKRDLPKLEGPMSTICQRNPWFKNTVDSFEIIEDRSRIVHDDVAYLVEFASVCIHPGVYLNWLLSQCLSLGATVVKRRVNHIKDANLLHSSGSRPDVIVNCSGLFARFLGGVEDKKLYPIRGQVVLVRNSLPFMAMFSSTPEKENEDEMLVIMTRFDGTSIIGGCFQPNNWSSEPDPSLTHRILSRALDRFPELTKDGPLDIVRECVGHRPGREGGPRVELEKIPGVGFVVHNYGAAGAGYQSSYGMADEAVSYVERALTRPNL